SEQUENCE LISTING (14 sequences)

SEQ ID NO: 1 - DNA, 33 bp, Artificial Sequence
  Synthetic oligonucleotide
ggccggatccatgatattttcaaagggtttagc

SEQ ID NO: 2 - DNA, 31 bp, Artificial Sequence
  Synthetic oligonucleotide
ggccctcgagctattttgttcttgtgatgac

SEQ ID NO: 3 - DNA, 32 bp, Artificial Sequence
  Synthetic oligonucleotide
aattactagtatggctctggtttctgctgtcc

SEQ ID NO: 4 - DNA, 34 bp, Artificial Sequence
  Synthetic oligonucleotide
ggttaattaactacaaaggcacagtttcaaccac

SEQ ID NO: 5 - DNA, 33 bp, Artificial Sequence
  Synthetic oligonucleotide
ggccactagtatggctcttaatctgctatcttc

SEQ ID NO: 6 - DNA, 37 bp, Artificial Sequence
  Synthetic oligonucleotide
ggttaattaattaaggaatgggatcaataaataaaga

SEQ ID NO: 7 - DNA, 31 bp, Artificial Sequence
  Synthetic oligonucleotide
ggccactagtatggctcttgtttctatcttg

SEQ ID NO: 8 - DNA, 32 bp, Artificial Sequence
  Synthetic oligonucleotide
ggttaattaattacaaaggcacagactcaagg

SEQ ID NO: 9 - DNA, 33 bp, Artificial Sequence
  Synthetic oligonucleotide
ggccactagtatgtcttcttgcattaatccctc

SEQ ID NO: 10 - DNA, 33 bp, Artificial Sequence
  Synthetic oligonucleotide
ggttaattaatcagcctttggtgccaggagatg

SEQ ID NO: 11 - DNA, 30 bp, Artificial Sequence
  Synthetic oligonucleotide
aattactagtatggctcttaatctgctctc

SEQ ID NO: 12 - DNA, 36 bp, Artificial Sequence
  Synthetic oligonucleotide
ggttaattaattaagcaatgggatcaaaaaataagg

SEQ ID NO: 13 - DNA, 34 bp, Artificial Sequence
  Synthetic oligonucleotide
ggccactagtatgtcttccattagcataaacata

SEQ ID NO: 14 - DNA, 34 bp, Artificial Sequence
  Synthetic oligonucleotide
ggttaattaatcagacataaggctggaatagcag

(19) United States
(12) Patent Application Publication
(10) Pub. No.: US 2013/0130345 A1
(43) Pub. Date: May 23, 2013

(54) PRODUCTION OF RENEWABLE AROMATIC COMPOUNDS

(71) Applicants: Wendy Thai, Champaign, IL (US); David A. Sikkenga, Wheaton, IL (US); Will Schroeder, Champaign, IL (US)

(72) Inventors: Wendy Thai, Champaign, IL (US); David A. Sikkenga, Wheaton, IL (US); Will Schroeder, Champaign, IL (US)

(73) Assignee: JNF BIOCHEMICALS, LLC, CHAMPAIGN, IL (US)

(21) Appl. No.: 13/807,302

(22) PCT Filed: Jun. 28, 2011
(86) PCT No.: PCT/US11/42067

§ 371 Date: Dec. 28, 2012

Related U.S. Application Data
(60) Provisional application No. 61/359,335, filed on Jun. 28, 2010.
(60) Provisional application No. 61/366,965, filed on Jul. 23, 2010.

(51) Int. Cl. C07C 6/00

(52) U.S. Cl. CPC C07C 6/00
USPC 435/166; 585/319; 585/470; 562/412

(57) ABSTRACT

The invention provides a process for producing a variety renewable aromatic compounds such as benzene, toluene, xylenes, and cumene, as well as compounds derived from these including, for example, aniline, benzoic acid, cresol, cyclohexane, cyclohexanone, phenol and bisphenol A, toluene di-isocyanate, isophthalic acid, phthalic anhydride, terephthalic acid and dimethyl terephthalate. The invention also provides for renewable forms of these aromatic compounds.

### BACKGROUND

Fossil fuels such as coal, petroleum and natural gas are rich sources of many industrial chemicals including the olefins such as ethylene, propylene, and butadiene; aromatic hydrocarbons such as benzene, toluene, and xylenes; and synthesis gas composed of varying amounts of carbon monoxide and hydrogen. Because fossil fuels come from the fossilized remains of plants and animals, they are non-renewable resources that are being depleted faster than they are being formed under present rate of consumption. In addition, the production and use of fossil fuels raise environmental issues including the release of large amounts of carbon dioxide.

### SUMMARY OF THE INVENTION

The invention provides processes for producing a variety of aromatic compounds from renewable resources. The invention is based on the discovery that biologically produced cyclic monoterpenes can be converted to cymene, which in turn can be used as a renewable feedstock to produce renewable cumene, renewable toluene and renewable forms of a variety of related aromatic compounds, examples of which include: (1) renewable phenol and acetone, as well as their condensation product Bisphenol A; (2) renewable toluene di-isocyanate; (3) renewable xylenes, as well as the isophthalic acid, phthalic anhydride and terephthalic acid derived from the xylene isoforms; (4) renewable benzene, cyclohexane and cyclohexanone, as well as a variety of alkylated benzenes having one or more methyl, isopropyl, or methyl and isopropyl substituents, including, without limitation renewable toluene, renewable cumene, renewable cymene, and renewable di-isopropyl benzene. Thus, the invention provides processes for producing renewable cumene, renewable toluene and a variety of aromatic compounds, hereinafter the “compounds of the invention,” as well as renewable cumene, renewable toluene, renewable benzene and renewable forms of a variety of aromatic compounds.

In one embodiment, the invention provides a process for producing a renewable aromatic compound that involves contacting a renewable cyclic monoterpene with a dehydrogenation catalyst under conditions effective for the oxidation of the cyclic monoterpene to renewable ρ-cymene and hydrogen (H₂,), and contacting the renewable ρ-cymene with benzene and a transalkylation catalyst under conditions effective for the transalkylation of the benzene with the renewable ρ-cymene to produce renewable cumene and renewable toluene.

In another embodiment, the invention provides a process for producing a renewable aromatic compound that involves: (a) cultivating a cell that comprises a monoterpene synthase under conditions effective to produce a cyclic monoterpene; (b) isolating at least a portion of the cyclic monoterpene, (c) contacting the cyclic monoterpene with a dehydrogenation catalyst under conditions effective for the oxidation of the cyclic monoterpene to ρ-cymene and H₂; (d) isolating at least a portion of the ρ-cymene; and (e) contacting the ρ-cymene with benzene in the presence of a transalkylation catalyst under conditions effective for the transalkylation of benzene with ρ-cymene to produce cumene and toluene.

In another embodiment, the invention provides a process for producing a renewable aromatic compound that involves: (a) cultivating a cell that comprises (i) a monoterpene synthase and (ii) a dehydrogenase or oxidase under conditions effective to produce ρ-cymene; (b) isolating at least a portion of the ρ-cymene; and (c) contacting the ρ-cymene with benzene in the presence of a transalkylation catalyst under conditions effective for the transalkylation of benzene with ρ-cymene to produce cumene and toluene.

In some embodiments, the cell is a fungal cell, e.g. a yeast cell. In some embodiments, the cell is a Schizosaccharomyces, Piromyces, Saccharomyces, Pichia, Hansenula, Kluyveromyces, Candida, or Debaryomyces. In some embodiments, the cell over expresses at least one gene in the mevalonic acid pathway, at least one gene in the non-mevalonic acid pathway, or at least one gene in the mevalonic acid and at least one gene in the non-mevalonic acid pathways. In some embodiments, the cell over expresses a gene encoding pyruvate dehydrogenase, a gene encoding HMG-CoA reductase or a gene encoding deoxy-xylulose phosphate synthase. In some embodiments, the cell overexpresses one or more genes encoding pyruvate dehydrogenase (aceE), HMG-CoA reductase (mvaA), deoxy-xylulose phoshate synthase (dxs), diphosphocytidylmethyl erythritol synthase (ispD), methyl-erythritol cyclodiphosphate synthase (ispF), and isopentenyl pyrophosphate isomerase (idi).

In some embodiments, the monoterpene synthase is a plant, fungal or bacterial enzyme. In some embodiments, the monoterpene synthase is an enzyme from Salvia officinalis, Citrus limon, Pinus taeda, Abies grandis, Citrus unshiu. In some embodiments, the monoterpene synthase is a limonene synthase, α-pinene synthase, β-pinene synthase, terpinene synthase, terpinolene synthase or sabinene synthase. In some embodiments, the dehydrogenase is a plant, fungal or bacterial enzyme. In some embodiments, the dehydrogenase is a yeast enzyme. In some embodiments, the oxidase is galactose oxidase.

In another embodiment, the invention provides a process for producing a renewable aromatic compound that involves contacting benzene with cymene and a transalkylation catalyst under conditions effective for the transalkylation of benzene with cymene to produce cumene and toluene, wherein the benzene comprises renewable benzene wherein all the carbons are renewable carbons, and the cymene comprises renewable cymene wherein all the carbons are renewable carbons.

In another embodiment, the invention provides a process for producing a renewable aromatic compound that involves: (a) contacting non-renewable benzene with renewable cymene and a first transalkylation catalyst under conditions effective for the transalkylation of the benzene with the cymene to produce renewable cumene and renewable toluene; (b isolating at least a portion of the renewable toluene; (c) contacting the renewable toluene with H₂ under conditions effective to produce renewable benzene; (d) isolating at least a portion of the renewable benzene; and (e) contacting the renewable benzene with renewable cymene and a second transalkylation catalyst under conditions effective for the transalkylation of the renewable benzene with the renewable cymene to produce renewable cumene and renewable toluene. In some embodiments, the renewable benzene of step (d) is combined with the non-renewable benzene of step (a) prior to contact with renewable cymene and the transalkylation catalyst. In some embodiments, the first and second transalkylation catalysts are the same catalyst. In some embodiments, the first and second transalkylation are performed in the same reactor.

In another embodiment, the invention provides a process for producing a renewable aromatic compound that involves: (a) providing a stream comprising a cyclic monoterpene; (b) passing the stream of step (a) to a dehydrogenation unit, wherein the stream is contacted with a dehydrogenation catalyst under conditions effective for the oxidation of the cyclic monoterpene to produce H₂ and a dehydrogenation-product stream comprising ρ-cymene; (c) separating the dehydrogenation-product stream of step (b) in a fractionation zone comprising at least one separation column to produce a fractionated ρ-cymene stream and a cyclic monoterpene stream; (d) combining the fractionated ρ-cymene stream of step (c) with benzene to produce a stream comprising benzene and ρ-cymene; (e) passing the stream comprising benzene and ρ-cymene of step (d) to a transalkylation unit, wherein the benzene and ρ-cymene is contacted with a transalkylation catalyst under conditions effective to produce a transalkylation-product stream comprising cumene; (f) separating the transalkylation-product stream of step (e) in a benzene separation column to produce a benzene-rich stream and a C₇₊-enriched stream; (g) separating the C₇₊-enriched stream of step (f) in a toluene separation column to produce a renewable toluene-enriched stream and a C₈₊-enriched stream; (h) separating the C₈₊-enriched stream of step (g) in a xylene separation column to produce a renewable xylene-enriched stream and a C₉₊-enriched stream; (i) separating the C₉₊-enriched stream of step (h) in a cumene separation column to produce a renewable cumene-enriched stream and C₁₀₊-enriched stream; and (j) separating the C₁₀₊-enriched stream of step (i) in a cymene separation column to produce a cymene-enriched stream and a C₁₀₊-enriched stream. In some embodiments, the process also involves mixing the benzene-rich stream of step (f) with the benzene and fractionated ρ-cymene stream of step (d) produce the stream comprising benzene and ρ-cymene. In some embodiments, the process also involves combining at least a portion of the transalkylation-product stream of step (e) with another transalkylation-product stream prior to separating the transalkylation product stream in the benzene separation column. In some embodiments, the process also includes passing the toluene-rich stream of step (g), or the xylene-rich stream of step (h), to a hydrodealkylation unit, wherein the streams are contacted with H₂, or H₂ and a hydrodealkylation catalyst, under conditions effective to produce renewable methane and renewable benzene. In some embodiments, the toluene-rich stream of step (g) is passed to a disproportionation unit, wherein the stream is contacted with a transalkylation catalyst under conditions effective to produce renewable xylenes and renewable benzene by toluene disproportionation.

In some embodiments of the invention, the cyclic monoterpene includes a cyclohexane, cyclohexene or cyclohexadiene ring. In some embodiments, the cyclic monoterpene is limonene, terpinene, pinene, terpinolene, sabinene or cineole. The cyclic monoterpene can be 4S-limonene, γ-terpinene or β-pinene. In some embodiments, the cyclic monoterpene is produced by bacterial fermentation or fungal fermentation. In some embodiments, the cyclic monoterpene is extracted from the fermentation medium using an organic solvent, centrifugation or distillation, e.g. steam distillation. In some embodiments, the organic solvent is acetone, hexane or liquid CO₂. In some embodiments, the monoterpene is produced by bacterial fermentation using an Escherichia, Pseudomonas or Bacillus species. In some embodiments, the monoterpene is produced by fungal fermentation using a yeast. In some embodiments, the yeast is a Schizosaccharomyces, Piromyces, Saccharomyces, Pichia, Hansenula, Kluyveromyces, Candida, or Debaryomyces. In some embodiments, the cyclic monoterpene is isolated from citrus rind, e.g. orange peels, or citrus processing wastes.

In some embodiments, the cymene is ρ-cymene. In some embodiments of the invention, the benzene includes renewable benzene and may also include non-renewable benzene. In some embodiments, the non-renewable benzene is in excess of the renewable benzene, or the renewable benzene is in excess of the non-renewable benzene. In some embodiments, the benzene includes non-renewable benzene. In some embodiments, the molar ratio of benzene to cymene is about 1:1 to about 50:1, about 2:1 to about 20:1, or about 5:1 to about 10:1.

In some embodiments, the transalkylation catalyst is an acid catalyst; a zeolite; a zeolite of the faujasite structure in the hydrogen form, a hydrogen mordenite, or another hydrogen zeolite with pore diameter of about 5.2 to about 7.8 Angstrom. The transalkylation catalyst can include an inorganic oxide binder, a zeolitic aluminosilicate selected from the group consisting of MTW, MFI, type Y, beta, and mordenite, and an optional metal component. In some embodiments, the transalkylation catalyst is a dealuminated HY zeolite or at least one of H+Beta, MCM-22, MCM-49, USY, SSZ-26, Al-SSZ-33, CIT-1, SSZ-35 or SSZ-44. In some embodiments, the transalkylation is carried out at a temperature from about 100° C. to about 540° C., a pressure from about 1 to about 90 kg/cm², and a weight hourly space velocity from about 0.1 to about 20 hr⁻¹. In one embodiment, the transalkylation is performed at a temperature from about 250° C. to about 500° C., a pressure from about 10 to about 65 kg/cm², and a weight hourly space velocity from about 1.0 to about 10 hr⁻¹. In some embodiments, the benzene, cymene and transalkylation catalyst are contacted under at least partial liquid phase conditions. In some embodiments, the cymene is depleted in the transalkylation.

In some embodiments of the invention, the dehydrogenation catalyst is a dehydrogenase, oxidase or a metal catalyst. The metal catalyst can be nickel, platinum, palladium, cobalt, cadmium, another noble metal, or a mixture thereof. In some embodiments, the process involves removing from the dehydrogenation reaction zone at least a portion of the H₂ as it is produced during the oxidation of the cyclic monoterpene to cymene. In some embodiments, the oxidation of the cyclic monoterpene to cymene and the transalkylation of benzene with cymene are performed in a single reactor. The single reactor can have at least two catalysts, at least one of which is a dehydrogenation catalyst, and at least one of which is a transalkylation catalyst. The dehydrogenation catalyst and the transalkylation catalyst can be parts of a multiple-function catalyst. The multiple-function catalyst can have surface oxidation sites that catalyze the oxidation of the cyclic monoterpene to renewable cymene and acidic sites in geometrically confined pores with about 5 Angstrom to about 7 Angstrom openings that catalyze the transalkylation of benzene with cymene.

In some embodiments, the renewable toluene or renewable cumene is isolated from the transalkylation product mixture. The renewable toluene can be contacted with H₂, or H₂ and a hydrodealkylation catalyst, under conditions effective to produce renewable methane and renewable benzene. The renewable toluene can be contacted with H₂ at 650-760° C. to produce renewable benzene and renewable methane. The hydrodealkylation catalyst can be a nickel, iron, chromium, molybdenum or rhodium catalyst; a platinum oxide catalyst; or a mixture thereof. In one embodiment, the hydrodealkylation is performed at a temperature of about 350° C. to about 700° C. and a pressure of about 5 to 100 atmospheres. In one embodiment, the hydrodealkylation is performed at a temperature of about 450° C. to about 650° C. and a pressure of about 15 to about 70 atmospheres. In some embodiments, the H₂ obtained from oxidation of a monoterpene is used for hydrodealkylation of toluene or xylene. In some embodiments, at least a portion of the methane is recovered from the hydrodealkylation reaction.

In some embodiments, the renewable toluene is contacted with a transalkylation catalyst under conditions effective to produce renewable xylenes and renewable benzene by toluene disproportionation. The transalkylation catalyst can be a mordenite or zeolite catalyst. The modenite catalyst can have silica to alumina mole ratios from about 5 to about 61. The transalkylation catalyst can be shape selective resulting in production of p-xylene in excess of the equilibrium concentration. In some embodiments, the catalyst is ZSM-5 in the hydrogen form, a partially metal ion exchanged ZSM-5, the hydrogen form of another zeolite with the MEI structure, or a partially metal ion exchanged form of another zeolite with the MEI structure. The disproportionation can be at about 200° C. to about 800° C.,—e.g. about 370° C. to about 500° C., about 450° C. to about 650° C., or about 400° C. to about 500° C. In one embodiment, the disproportionation is at about 1 to about 100 atmospheric pressures. In one embodiment, the disproportionation is performed in a fixed bed reactor at a temperature of about 350° C. to about 500° C. and a pressure of about 20 to about 50 kg/cm². The disproportionation can be performed in the presence of nitrogen or hydrogen gas.

In some embodiments, at least a portion of the renewable xylenes is recovered. The renewable xylenes can be separated into m-xylene, o-xylene and ρ-xylene. In one embodiment, the renewable m-xylene is contacted with O₂ under conditions effective for the oxidation of the m-xylene to renewable isophthalic acid. In one embodiment, the renewable o-xylene is contacted with O₂ in the presence of a catalyst under conditions effective for the oxidation of the o-xylene to renewable phthalic anhydride. In one embodiment, the renewable ρ-xylene is contacted with O₂ in the presence of a catalyst under conditions effective for the oxidation of ρ-xylene to renewable terephthalic acid. In one embodiment, the renewable ρ-xylene is contacted with O₂ in the presence of a catalyst under conditions effective for the oxidation of the ρ-xylene to produce an oxidate comprising ρ-toluic acid and monomethyl terephthalate, and then the oxidate is contacted with methanol under conditions effective for esterification to produce renewable dimethyl terephthalate. In some embodiments, the oxidation of xylene is performed in acetic acid solvent. In some embodiments, the catalyst is a cobalt-manganese catalyst. In some embodiments, the oxidation involves a promoter, which can be bromide. In some embodiments, the renewable terephthalic acid is contacted with methanol under conditions effective for the esterification of terephthalic acid with methanol to produce renewable dimethyl terephthalate.

In one embodiment, at least a portion of the renewable benzene produced by a process of the invention is isolated. In one embodiment, the renewable benzene is contacted with H₂ under conditions effective for the reduction of benzene to renewable cyclohexane. The H₂ can come from oxidation of a monoterpene. In one embodiment, the renewable cyclohexane is contacted with oxygen (O₂) in the presence of a catalyst under conditions effective for the oxidation of the cyclohexane to renewable cyclohexanone. The catalyst can be a cobalt catalyst. In some embodiments, the renewable benzene is contacted with propylene, oxygen and a catalyst under conditions effective to produce acetone and renewable phenol. In one embodiment, the benzene and propylene are contacted at about 150° C. to about 400° C. and about 5 to about 70 standard atmospheric pressures. In some embodiments, the renewable cumene of the invention is contacted with oxygen and a catalyst under conditions effective to produce renewable acetone and renewable phenol. The catalyst can be phosphoric acid, a strong acid ion exchange resin, or a zeolite in the hydrogen form. In one embodiment, the renewable phenol of the invention is contacted with renewable acetone under conditions effective for the condensation of phenol with acetone to produce renewable Bisphenol-A. In some embodiment, the renewable phenol is contacted with formaldehyde under conditions effective for the condensation of the phenol with formaldehyde to produce a renewable phenolic resin. In some embodiments, the renewable benzene is contacted with nitric acid and sulfuric acid under conditions effective to produce nitrobenzene, which is then contacted with a metal catalyst under condition effective for the hydrogenation of nitrobenzene to renewable aniline. The benzene can be contacted with nitric acid and sulfuric acid at 50° C. to 60° C. The hydrogenation of nitrobenzene to aniline can be performed at 200° C. to 300° C.

In one embodiment, the renewable toluene is contacted with nitric acid in the presence of a catalyst under conditions effective to form dinitrotoluene. The dinitrotoluene is then contacted with hydrogen in the presence of a hydrogenation catalyst under conditions effective to form toluene diamine. At least a portion of meta-toluene diamine is isolated and contacted with phosgene under conditions effective to produce a renewable toluene diisocyanate mixture. In some embodiments, the renewable toluene diisocyanate mixture of step is distilled to obtain a mixture of 2,4-toluene diisocyanate and 2,6-toluene diisocyanate. The 2,4-toluene diisocyanate can be separated from 2,6-toluene diisocyanate to obtain pure renewable 2,4-toluene diisocyanate. In some embodiments, the meta-toluene diamine and phosgene are in gas form when contacted. In one embodiment, the renewable toluene is contacted with O₂ under conditions effective for the oxidation of the toluene to form renewable benzoic acid. In one embodiment, the renewable benzene produced by a process of the invention is contacted with nitric acid and sulfuric acid under conditions effective to produce nitrobenzene, with is then contacted with a metal catalyst under condition effective for the hydrogenation of nitrobenzene to renewable aniline.

In some embodiments, the invention provides a solvent, cleaning agent or thinner for paint or varnish that includes a renewable or toluene of the invention. The solvent can be used in printing, rubber and leather applications, the cleaning agent can be used on steel, silicon wafers and chips. In some embodiments, the renewable xylene or renewable toluene can be included in gasoline or jet fuel.

In another embodiment, the invention provides a process for producing renewable cresol that involves contacting a renewable cyclic monoterpene with a dehydrogenation catalyst under conditions effective for the oxidation of the cyclic monoterpene to ρ-cymene and contacting the ρ-cymene with oxygen and a catalyst under conditions effective for the oxidation of the ρ-cymene to cresol. In another embodiments, the invention provides a process for producing renewable cresol that includes: (a) cultivating a cell that has a monoterpene synthase under conditions effective to produce a cyclic monoterpene; (b) isolating at least a portion of the cyclic monoterpene, (c) contacting the cyclic monoterpene with a dehydrogenation catalyst under conditions effective for the oxidation of the cyclic monoterpene to ρ-cymene and H₂; (d) isolating at least a portion of the ρ-cymene; and (e) contacting the ρ-cymene with oxygen and a catalyst under conditions effective for the oxidation of ρ-cymene to cresol. In another embodiment, the invention provides a process for producing renewable cresol that involves: (a) cultivating a cell that has (i) a monoterpene synthase and (ii) a dehydrogenase or oxidase under conditions effective to produce ρ-cymene; (b) isolating at least a portion of the ρ-cymene; and (c) contacting the ρ-cymene with oxygen and a catalyst under conditions effective for the oxidation of ρ-cymene to cresol. In some embodiments, the ρ-cymene can be contacted with oxygen and an acidic catalyst or a vanadium phosphate catalyst for oxidation of ρ-cymene to cresol. Thus, the invention provides for renewable o-, m- and p-cresol.

In another embodiment, the invention provides a process for producing renewable terephthalic acid that involves contacting a renewable cyclic monoterpene with a dehydrogenation catalyst under conditions effective for the oxidation of the cyclic monoterpene to renewable cymene and contacting the ρ-cymene with a catalyst under conditions effective for the oxidation of ρ-cymene to terephthalic acid. In another embodiment, the invention provides a process for producing terephthalic acid that involves: (a) cultivating a cell that has a monoterpene synthase under conditions effective to produce a cyclic monoterpene; (b) isolating at least a portion of the cyclic monoterpene, (c) contacting the cyclic monoterpene with a dehydrogenation catalyst under conditions effective for the oxidation of the cyclic monoterpene to ρ-cymene and H₂; (d) isolating at least a portion of the ρ-cymene; and (e) contacting the ρ-cymene with a catalyst under conditions effective for the oxidation of ρ-cymene to terephthalic acid. In another embodiment, the invention provides a process for producing a renewable aromatic compound that involves: (a) cultivating a cell that has (i) a monoterpene synthase and (ii) a dehydrogenase or oxidase under conditions effective to produce ρ-cymene; (b) isolating at least a portion of the ρ-cymene; and (c) contacting the p-cymene with a catalyst under conditions effective for the oxidation of ρ-cymene to terephthalic acid. In some embodiments, the catalyst includes cobalt, manganese, bromine, or a combination thereof. In some embodiments, the ρ-cymene is contacted with a catalyst in the presence of acetic acid.

In one embodiment, the invention provides renewable cumene in which the carbons in the isopropyl substituent are renewable carbons or renewable cumene wherein all the carbons are renewable carbons. The invention also provides purified cumene that includes at least one renewable form of cumene. The proportion of radiocarbon to total carbon in the purified cumene is greater than that of similarly pure non-renewable cumene. The proportion of radiocarbon to total carbon in the purified cumene can correspond to a renewable carbon content of at least about 65%, e.g. about 70%, about 75%, about 80%, about 85%, about 90%, about 95% or about 100%. The purified cumene can also include non-renewable cumene.

As used herein the term “purified” refers to a composition that has been processed using at least one method of separation known to those of skill in the art such that composition is enriched for a selected chemical compound. A purified substance is a composition composed predominantly of the substance though impurities such as contaminants may be present. Purified cumene, toluene, benzene or xylene, for example, is a composition composed predominantly of cumene, toluene, benzene or xylene, respectively. Such compositions can include about 80%, about 85%, about 90%, about 95%, about 98% or more than about 98% cumene, toluene, benzene or xylene. Methods of separation can be based on size, solubility, charge, melting or boiling point, selective chemical reactivity or any other physical attributes that can be used to distinguish one chemical compound from another. Methods of separation include, without limitation, dessication, centrifugation, crystallization, precipitation, solvent extraction, distillation, decanting, and phase partitioning. Methods of separation also can include selective chemical reactions or complex formation such as, for example, the use of phenanthrene, 7,8 benzoquinoline, 1,8-diaminonaphthalene, and m-phenylenediamine for the separation of m- and ρ-xylene isomers. A purified substance can be a mix of renewable and non-renewable forms of the substance, as well as a mix of different renewable forms of the substances. Thus, purified cumene can be a mix of renewable or nonrenewable cumene, as well as a mix of different renewable forms of cumene.

In another embodiment, the invention provides renewable toluene in which the carbon in the methyl substituent is a renewable carbon or renewable toluene in which all of the carbons are renewable carbons. The invention also provides toluene that includes one or more renewable forms of toluene. The proportion of radiocarbon to total carbon in renewable toluene or purified toluene is greater than that of a similar non-renewable toluene or similarly pure non-renewable toluene and correspond to renewable carbon content of at least about 55%, e.g. about 60%, about 65%, about 70%, about 75%, about 80%, about 85%, about 90%, about 95% or about 100%. Toluene or purified toluene can include non-renewable toluene.

In another embodiment, the invention provides benzene that is composed of renewable benzene in which all of the carbons are renewable carbons and non-renewable benzene. The invention also provides purified benzene that includes a renewable form of benzene. The proportion of radiocarbon to total carbon in the purified benzene or benzene that includes renewable benzene is greater than that of similarly pure non-renewable benzene. The proportion of radiocarbon to total carbon in the purified benzene can correspond to a renewable carbon content of at least about 50%, e.g., at least about 55%, about 60%, about 65%, about 70%, about 75%, about 80%, about 85%, about 90%, about 95% or about 100%. The purified benzene can include non-renewable benzene.

In another embodiment, the invention provides a mixed aromatic composition that includes purified benzene and purified renewable cymene, e.g. ρ-cymene, m-cymene or o-cymene. The benzene can be renewable or non-renewable. The benzene can be in molar excess of the cymene, e.g. a molar ratio of benzene to cymene of about 1:1 to about 50:1, e.g. 2:1 to about 20:1. The proportion of radiocarbon to total carbon in the composition is greater than that of a similar composition that includes purified, non-renewable benzene and purified, non-renewable cymene. The composition can include a transalkylation catalyst such as a zeolite catalyst, e.g. zeolite beta.

In another embodiment, the invention provides a mixed aromatic composition that has renewable benzene, renewable cymene, renewable toluene and renewable cumene. The proportion of radiocarbon to total carbon in the composition is greater than that of a similarly pure aromatic composition that includes similar amounts of non-renewable benzene, non-renewable cymene, non-renewable toluene and non-renewable cumene.

In another embodiment, the invention provides renewable xylenes (m-, o- and p-isomers) in which the carbons in the methyl substituents are renewable carbons or all the carbons in the xylene are renewable carbons. The invention also provides purified xylene of any one isomer or any combination thereof. The carbons in the methyl substituents of xylenes are renewable carbons or all the carbons in the xylenes are renewable carbons. The proportion of radiocarbon to total carbon in the purified xylene is greater than that of similarly pure non-renewable xylene. The proportion of radiocarbon to total carbon in the purified xylene corresponds to a renewable carbon content of at least about 60%, e.g. about 65%, about 70%, about 75%, about 80%, about 85%, about 90%, about 95% or about 100%. The purified xylene can include non-renewable xylene.

In another embodiment, the invention provides renewable toluene di-isocyanate. in which the carbon in the methyl substituent is a renewable carbon or the carbons in the methyl substituent and the phenyl ring are renewable carbons. The invention also provides purified toluene di-isocyanate that contains a renewable form of toluene di-isocyanate such that the proportion of radiocarbon to total carbon in the purified toluene di-isocyanate is greater than that of similarly pure non-renewable toluene di-isocyanate, e.g. it corresponds to a renewable carbon content of at least about 40% and less than about 79%.

In another embodiment, the invention provides renewable cyclohexane in which all the carbons are renewable carbons. The invention also provides purified cyclohexane in which the proportion of radiocarbon to total carbon in the purified cyclohexane is greater than that of similarly pure non-renewable cyclohexane, e.g. the proportion of radiocarbon to total carbon in the purified cyclohexane corresponds to a renewable carbon content of at least about 50%. The purified cyclohexane includes a renewable form of cyclohexane, it can also include non-renewable cyclohexane.

In another embodiment, the invention provides renewable cyclohexanone in which all the carbons are renewable carbons. The invention provides purified cyclohexanone that includes a renewable form of cyclohexanone. The proportion of radiocarbon to total carbon in the purified cyclohexanone is greater than that of similarly pure non-renewable cyclohexanone, e.g. the proportion of radiocarbon to total carbon in the purified cyclohexanone corresponds to a renewable carbon content of at least about 50%. The purified cyclohexanone can also include non-renewable cyclohexanone.

In another embodiment, the invention provides renewable isophthalic acid in which the carbons in the carboxyl substituents are renewable carbons or all the carbons are renewable carbons. The invention also provides purified isophthalic acid that includes a renewable form of isophthalic acid. The purified isophthalic acid can also include non-renewable isophthalic acid. The proportion of radiocarbon to total carbon in the purified isophthalic acid is greater than that of similarly pure non-renewable isophthalic acid, e.g. the proportion of radiocarbon to total carbon in the purified isophthalic acid corresponds to a renewable carbon content of at least about 60%.

In another embodiment, the invention provides renewable phthalic anhydride in which the carbons in the anhydryl group are renewable carbons or all the carbons are renewable carbons. The invention also provides purified phthalic anhydride that contains a renewable form of phthalic anhydride. The proportion of radiocarbon to total carbon in the purified phthalic anhydride is greater than that of similarly pure non-renewable phthalic anhydride composition, e.g. the proportion of radiocarbon to total carbon in the purified phthalic anhydride corresponds to a renewable carbon content of at least about 60%. The purified phthalic anhydride can include non-renewable phthalic anhydride. In another embodiment, the invention provides renewable aniline, renewable benzoic acid, renewable dimethyl terephthalate, and renewable cresol. In another embodiment, the invention provides renewable dimethyl terephthalate and renewable terephthalic produced according to a method of the invention.

In another embodiment, the invention provides a process for producing a renewable aromatic compound that includes: (a) providing a stream comprising a cyclic monoterpene; (b) passing the stream of step (a) to dehydrogenation unit, wherein the stream is contacted with a dehydrogenation catalyst under conditions effective for the desaturation of the cyclic monoterpene to produce a dehydrogenation product stream comprising ρ-cymene; (c) separating the product stream of step (b) in a fractionation zone comprising at least one separation column to produce a fractionated-cymene stream and a cyclic monoterpene stream; (d) combining the fractionated-cymene stream of step (c) with benzene to produce a stream comprising ρ-cymene and benzene; (e) passing the benzene and ρ-cymene stream of step (d) to a transalkylation unit, wherein the benzene-cymene stream is contacted with a transalkylation catalyst under conditions effective to produce a transalkylation product stream comprising cumene; (f) separating the transalkylation product stream of step (e) in a benzene separation column to produce a benzene-rich stream and a C₇₊ enriched stream; (g) separating the C₇₊ enriched stream of step (e) in a toluene separation column to produce a toluene enriched stream and a C₈₊ enriched stream; (h) separating the C₈₊ enriched stream of step (e) in a xylene separation column to produce a xylene-enriched stream and a C₉+ enriched stream; (i) separating the C₉₊ enriched stream of step (h) in a cumene separation column to produce a cumene-enriched stream and a C₁₀₊-enriched stream; and (j) separating the C₁₀₊-enriched stream of step (i) in a ρ-cymene separation column to produce a cymene-enriched stream and a C₁₀₊-enriched stream.

In another embodiment, the invention provides a process for producing cumene or toluene that involves: (a) mixing a benzene-containing fluid with a renewable cymene-containing fluid to form a first mixed aromatic fluid; and (b) contacting the first mixed-aromatic fluid with a transalkylation catalyst under conditions effective for the transalkylation of benzene with cymene to form a second mixed aromatic fluid comprising cumene and toluene, in which the cymene-containing fluid comprises renewable cymene.

In another embodiment, the invention provides a process for producing benzene or xylene that involves contacting renewable toluene of the invention with a catalyst under conditions effective for disproportionation of toluene to produce renewable benzene and renewable xylene. In another embodiment, the invention provides a process for producing benzene that involves contacting renewable toluene of the invention, or renewable xylene of the invention, with H₂ under conditions effective for the hydrodealkylation of the toluene or xylene to produce benzene and renewable methane. In another embodiment, the invention provides a process for producing renewable benzoic acid that involves contacting a renewable toluene of the invention with oxygen under conditions effective for the oxidation of the renewable toluene to form renewable benzoic acid.

In another embodiment, the invention provides a process for producing phenol that involves contacting a renewable cumene of the invention with oxygen in the presence of a catalyst under conditions effective to produce renewable phenol and renewable acetone. In another embodiment, the invention provides a process for producing phenol that involves: (a) contacting benzene produced by a method of the invention with propylene in the presence of a catalysts underconditions effective for the alkylation of benzene by propylene to produce cumene, and (b) oxidizing the cumene under conditions effective to produce renewable phenol. The catalyst for use in producing phenol can be phosphoric acid, a strong acid ion exchange resin, or a zeolite in the hydrogen form. In another embodiment, the invention provides a method for producing bisphenol-A that involves contacting phenol of the invention with acetone under conditions effective for the condensation of phenol with acetone to produce bisphenol-A. In another embodiment, the invention provides a method of producing a phenolic resin that involves contacting a phenol of the invention with formaldehyde under conditions effective for the condensation of phenol with formaldehyde to produce a phenolic resin. In another embodiment, the invention provides a method for producing cyclohexanone that involves contacting the phenol produced by a method of the invention with H₂ under conditions effective for the hydrogenation of phenol to produce cyclohexanone.

In another embodiment, the invention provides a method for producing toluene di-isocyanate that involves (a) contacting toluene produced using a method of the invention with nitric acid in the presence of a catalyst under conditions effective to form dinitrotoluene, (b) contacting the dinitrotoluene with hydrogen in the presence of a hydrogenation catalyst under conditions effective to form toluene diamine, (c) isolating at least a portion of meta-toluene diamine, and (d) contacting the meta-toluene diamine with phosgene under conditions effective to produce a toluene diisocyanate mixture. In another embodiment, the invention provides a method for producing renewable dimethyl terephthalate that involves contacting terephthalic acid producing using a process of the invention with methanol under conditions effective for the esterification of terephthalic acid with methanol to produce dimethyl terephthalate.

Any feature or combination of features described herein are included within the scope of the present invention provided that the features included in any such combination are not mutually inconsistent as will be apparent from the context, this specification and the knowledge of one of ordinary skill in the art. Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents and other references mentioned herein are hereby incorporated by reference in their entirety. Amino acid designations may include full name, three-letter, or single-letter designations as commonly understood by one of ordinary skill in the art to which this invention belongs. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF THE FIGURES

FIG. 1 illustrates the major equipment used in performing a process of this invention. In the process, cyclic monoterpenes (C₁₀ compounds) carried by line 14 are admixed with recycled monoterpenes from line 26 to form a combined line 16 that enters a dehydrogenation reactor 18. After contact with a catalyst such as Raney Nickel, line 20 carries the effluent from the dehydrogenation reactor 18 to a separation column 24, while line 22 carries the H₂ generated to a hydrodealkylation reactor 66. Separation column 24 separates the effluent from the dehydrogenation reactor 18 into cymene, which is taken by line 28, and unconverted monoterpenes, which are removed by line 26. The monoterpenes in line 26 are recycled back to the dehydrogenation reactor 18 by line 16 after being combined with additional monoterpenes via line 14. The cymene in line 28 is taken to a combination point with a second cymene-containing distillation stream in line 56 (from separation column 54) to form a combined stream in a line 30 that enters a transalkylation reactor 32. After contact with a zeolitic catalyst in transalkylation reactor 32, a line 34 carries the effluent from the transalkylation reactor 32 to a separation column 36. Separation column 36 separates the effluent from the transalkylation reactor 32 into an overhead of benzene taken by line 40 and a bottom stream of C₇₊ alkylaromatics including toluene, xyene, cumene, cymeme and di-isopropyl benzene taken by line 38. The overhead stream of benzene in line 40 is taken to a combination point with benzene from a distillation stream in line 76 (from separation column 72) to form a combined stream in a line 80. The benzene in line 80 is recycled back to transalkylation reactor 32 by line 84 after the benzene is added or removed via line 82. The bottom stream of C₇₊ alkylaromatics in line 38 enters a separation column 42, which separates the C₇₊ alkylaromatics into an overhead of toluene taken by line 46 and a bottom stream of C₈₊ alkylaromatics including xylene, cumene, cymene and di-isopropylbenzene taken by line 44. The overhead stream of toluene in line 46 is either removed by line 64 or admixed with toluene and xylene from a distillation stream in line 74 (from separation column 72) and xylene from a distillation stream in line 60 to form a combined stream in line 78 that is sent to a hydrodealkylation reactor 66. The effluent from the hydrodealkylation reactor 66 is carried by line 70 to a distillation column 72, while the CH₄ is collected in line 68. Distillation column 72 separates effluent in line 70 into an overhead of benzene, which is carried by line 76, and a bottom stream of toluene and xylene, which is taken by line 74. The benzene in line 76 is combined with the benzene from line 40 and the resulting benzene in line 80 is recycled back to the transalkylation reactor 32 by line 84 after benzene is added or removed by line 82. The toluene and xylene in line 74 is recycled back to the hydrodealkylation reactor 66 after it is combined with the toluene from line 46 and the xylene from line 60. The bottom stream from the separation column 42 is carried by line 44 to another separation column 48 that separates the C₈₊ alkylaromatics into an overhead stream of xylene, which is taken by line 52, and a bottom stream of C₉₊ alkylaromatic including cumene, cymene and di-isopropylbenzene, which is taken by line 50. The overhead stream of xylene in line 52 is recovered by line 62 or taken by line 60 to a combination point where it is admixed with toluene from line 46 and toluene and xylene from line 74 to form a mixture that is carried to hydrodealkylation reactor 66. The bottom stream of C₉₊ alkylaromatic in line 50 is sent to another separation column 54, which separates the distillation stream in line 50 into an overhead stream of cumene recovered in line 58, and a bottom stream of C₁₀₊ alkylaromatics including cymene and di-isopropylbenzene taken by line 56. The C₁₀₊ alkylaromatic in line 56 is recycled to the transalkylation reactor 32 by line 30 after it is admixed with cymene from line 28. Details of heat exchange and additional flow details are not shown as they are well known to the art.

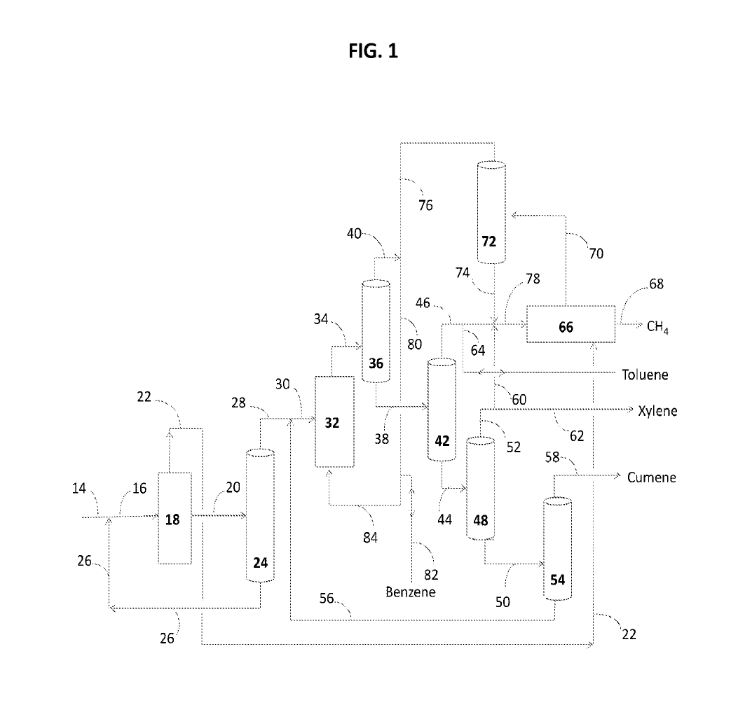

FIG. 2 summarizes the two pathways for the biosynthesis of limonene and identifies gene function that, when up or down regulated, can increase limonene biosynthesis.

FIG. 3 is a diagram of the equipments for recovery of monoterpene oil from fermentation broth.

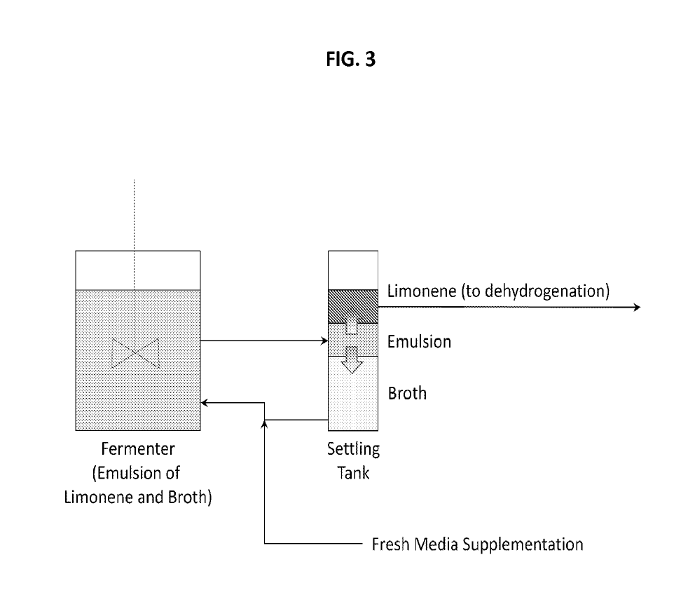

### DETAILED DESCRIPTION OF THE INVENTION

The invention relates to the production of renewable forms of a variety of aromatic compounds including renewable cumene, toluene, benzene and related aromatic compounds. The invention is based on the discovery of a method for producing cumene and toluene using renewable feedstock. More specifically, the processes of the invention involve the use of cymene obtained from renewable matter as an intermediate to produce a variety of renewable aromatic compounds including cumene, toluene, benzene and related aromatic compounds. According to the invention, renewable cymene can be used to produce renewable cumene and renewable toluene through a transalkylation reaction with benzene. Renewable cumene and renewable toluene can be used as feedstocks to produce renewable forms of a variety of related aromatic compounds including, without limitation: (1) renewable phenol and acetone, as well as their condensation product Bisphenol A; (2) renewable toluene di-isocyanate; (3) renewable xylenes, as well as the isophthalic acid, phthalic anhydride and terephthalic acid derived from the xylene isoforms; (4) benzene, cyclohexane and cyclohexanone, as well as a variety of alkylated benzenes having one or more methyl, isopropyl, or methyl and isopropyl substituents, including, without limitation renewable toluene, renewable cumene, renewable cymene, and renewable di-isopropyl benzene. Thus, the invention provides a variety of renewable aromatic compounds, i.e. cumene, toluene, as well as related aromatic compounds (herein renewable aromatic compounds of the invention), and processes for their production.

I. Renewable and Non-Renewable Matter

As used herein, the term “renewable” means from the biosphere, i.e. the zone of life on earth, or from biomass, i.e. biological material derived from living or recently living organisms. Renewable is distinguished from non-renewable, which means from fossil-derived matter such as petrochemicals and fossil fuels (e.g. coal, petroleum and natural gas). Renewable matter has a radiocarbon (¹⁴C) content greater than that found in non-renewable fossil-derived matter, as the latter, being millions of years old, contains no significant amounts of radiocarbon.

Thus, the term “renewable” means from living organisms including, without limitation: (1) trees such as, for example, conifers (e.g. fir, pine), and deciduous (e.g. poplar, maple, birch, eucalyptus aspen); (2) other plants such as, for example, basil, corn, wheat, barley, cotton, rice, guayule, Jerusalem artichoke, citrus fruit (e.g. oranges, grapefruit), mint, peppermint, spearmint, other herbs, potatoes, soybeans, sorghum, switch grass, thyme and tomatoes; (3) marine organisms such as, for example, algae, seaweed, reeds and rushes; (4) fungi such as Aspergillus niger, Blakeslea trispora, Phycomyces blakesleeanus, Ceratocystis moniliformis, Trametes odorata and Phellinus species; (5) other microorganisms such as, for example, yeast (e.g. Saccharomyces cerevisiae) and bacteria (e.g. Escherichia coli); (6) insects such as boll weevils, termites and mealybugs; and (7) animals such as alligators, beavers and pygmy three-toed sloths.

The term “renewable” can refer to parts of living things. Thus, renewable matter includes, without limitation: apple pomace, beet molasses, biomass, citrus processing waste, corn syrup, molasses, municipal wastes, sugar cane, yard wastes, wood, wood pulp, paper processing waste and charcoal. The term “renewable” can also refer to substances that come from living organisms, i.e. substances that are produced biologically. Non-limiting examples of renewable substances include, without limitation: resin and sap from trees, essential oils from plants, primary or secondary metabolites, and other macromolecules or chemical compounds. Non-limiting examples of renewable substances include the monoterpenes, monoterpenoids and related compounds (such as cymene) that are produced by a large variety of trees, plants, algae and animals.

The term “renewable” can also refer to chemical compounds that are composed of one or more carbons that come from renewable matter, i.e. one or more renewable carbons. Thus, in a renewable compound, all the carbons can be renewable carbons. For example, all the carbons in limonene recovered from a renewable source such as citrus rinds or from citrus processing wastes are renewable carbons. Similarly, all the carbons in limonene biosynthesized by an organism such as a plant, a yeast or bacterium are also renewable carbons. Furthermore, if biosynthesized limonene is aromatized to ρ-cymene by in vitro dehydrogenation as summarized in the reaction below, all the carbons in ρ-cymene are also renewable carbons as they come from limonene.

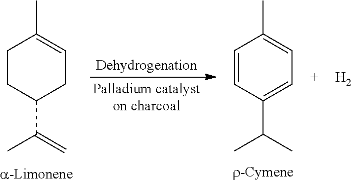

See §3.4 Conversion of Limonene to ρ-Cymene Using a Palladium Catalyst on Charcoal in LIMONENE PRACTICAL 8, at http://www.greenchemistrynetwork.org/pdf/LimonenePractical.pdf.

Renewable compounds in which all the carbons are renewable carbons, for example, the limonene and cymene discussed above, are said to have “renewable carbon contents” of 100%. In contrast, a non-renewable compound has a renewable carbon content of 0%, as it is derived from non-renewable fossil matters such as, without limitation, fossil fuels such as coal, petroleum and natural gas. Examples of non-renewable compounds include, without limitation, the olefins such as ethylene, propylene and butadiene that come from natural gas, Olefins can be produced by steam cracking of natural gas liquids such as ethane and propane or by fluid catalytic cracking of petroleum fractions. Non-renewable compounds also include petroleum or coal-derived aromatics such as benzene, toluene or xylene. These can be recovered from petroleum by fluid catalytic cracking, toluene can be recovered from crude light oil as a by-product of coke manufacturing.

A renewable compound can be composed of non-renewable carbons as long as it is composed of at least one renewable carbon. Thus, a renewable compound can have a combination of renewable and non-renewable carbons. A renewable compound that is composed of renewable and non-renewable carbons can be produced by transalkylation of a non-renewable compound with a renewable compound. For example, if renewable ρ-cymene (e.g. cymene isolated from the essential oils of basil or obtained by dehydrogenation of D-limonene extracted from orange peels) is subject to a transalkylation reaction with benzene recovered from fossil matter, the products can have renewable carbons (from ρ-cymene) and non-renewable carbons (from benzene) as shown below.

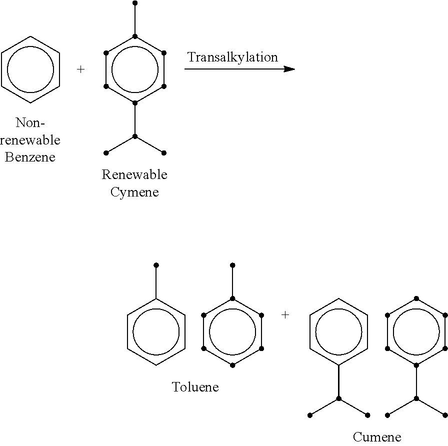

In the above reaction, the methyl and isopropyl substituents are composed of renewable carbons (indicated by •) as these substituents come from renewable cymene. In contrast, the phenyl groups in toluene and cumene products can be composed of non-renewable or renewable carbons depending on whether they come from non-renewable benzene or renewable cymene, respectively. Thus, each reaction product in the above reaction can occur in two “renewable forms,” one of which is composed of at least one renewable carbon and non-renewable carbons.

A compound that is composed of renewable and non-renewable carbons has a renewable carbon content that is greater than 0%, but less than 100%. The renewable carbon content is the number of renewable carbons to total carbon count (given as a percentage). A renewable carbon content of 10% indicates that 1 of 10 carbons are renewable, while renewable carbon content of 100% indicates that all the carbons are renewable. A renewable carbon content of 50% indicates that half of the carbons are renewable. In general, the renewable carbon content of a sample, whether it is biomass, substances derived from biomass, a chemical compound, a purified chemical, or a single molecule, is given by the formula:

$$Renewable Carbon Content = \frac{Number of Renewable Carbon ( s )}{Total Number of Carbons} \times 100 \%$$

The above illustrates two renewable forms of toluene: (1) renewable toluene in which only the carbon in methyl substituent is a renewable carbon (1 of 7 carbons is a renewable carbon), or (2) renewable toluene in which all the carbons are renewable carbons. As such, the first renewable form of toluene has a renewable carbon content of about 14%, and the second renewable form of toluene has a renewable carbon content of 100%. Similarly two renewable forms of cumene are shown above: (1) renewable cumene in which only the carbons in isopropyl substituent are renewable carbons (3 of 9 carbons are renewable carbons), or (2) renewable cumene in which all the carbons are renewable carbons. Therefore, the first renewable form of cumene has a renewable carbon content of about 33%, and the second renewable form of cumene has a renewable carbon content of 100%.

Toluene that is composed of equal amounts of the two renewable forms, has a renewable carbon content of about 57% (i.e. [0.5× about 14%]+[0.5×100%]=about 57%) since 8 of the 14 carbons are renewable carbons. Toluene that is composed of unequal amounts of the two forms can have a renewable carbon content between about 14% and about 99% depending on the amount of each renewable form. For example, toluene that is composed of about 20% of the first renewable form (about 14% renewable carbons) and about 80% of the second renewable form (100% renewable carbons) has a renewable carbon content of about 83% (i.e. [0.2× about 14%]+[0.8×100%]=about 82.8%). In contrast, toluene containing about 70% of the first renewable form (about 14% renewable carbons) and about 30% of the second renewable form (100% renewable carbons) has a renewable carbon content of about 40% (i.e. [0.7× about 14%]+[0.3×100%]=about 39.8%).

Cumene that is composed of equal amounts of the two renewable forms has a renewable carbon content of about 67% (i.e. [0.5× about 33%]+[0.5×100%]=about 66.5%) since 12 of the 18 carbons are renewable carbons. Cumene that is composed of unequal amounts of the two forms can have a renewable carbon content between about 33% and about 99% depending on the amount of each renewable form. For example, cumene that is composed of 20% of the first renewable form (33% renewable carbons) and 80% of the second renewable form (100% renewable carbons) has a renewable carbon content of about 87% (i.e. [0.2× about 33%]+[0.8×100%]=about 86.6%). In contrast, cumene containing about 70% of the first renewable form (about 33% renewable carbons) and about 30% of the second renewable form (100% renewable carbons) has a renewable carbon content of about 53% (i.e. [0.7× about 33%]+[0.3×100%]=about 53.1%). The renewable carbon content of a mix of two renewable forms (A & B) of a chemical compound can be determined as follows.

Renewable Carbon Content (in %) of a Mixture of Form A and Form B=a+b; where:

$$a = \% of A in blend \times \frac{Number of Renewable Carbon ( s ) in A}{Total Number of Carbons in A}$$

$$b = \% of B in blend \times \frac{Number of Renewable Carbon ( s ) in B}{Total Number of Carbons in B}$$

Thus, whether a chemical compound is renewable or non-renewable, its renewable carbon content, and the renewable carbon content of a mixture of two forms is based on the origin of the carbons in the chemical compound. A carbon that comes from renewable matter is a renewable carbon. A chemical compound that is composed of at least one renewable carbon is a renewable compound. Furthermore, whether a chemical compound is renewable or non-renewable and its renewable carbon content can be determined by radiocarbon analysis using methods known to those of skilled in the art including, for example, Liquid Scintillation Counting (LSC), Accelerator Mass Spectrometry (AMS), or Isotope Ratio Mass Spectrometry (IRMS).

Carbons come in several isotopic forms, for example, ¹²C, ¹³C and ¹⁴C. Of these, ¹²C and ¹³C are stable forms of carbon, while ¹⁴C (radiocarbon) is radioactive and has a half-life of about 5730 years. Because radiocarbon is continually produced in the atmosphere, it is continually incorporated into living organisms along with ¹²C. Thus, a constant portion of carbons in renewable matter are radiocarbons. In contrast, non-renewable matter is fossil-derived, and as such, it is millions of years old. Because radiocarbon will decay to undetectable levels after ten half lives (about 58,000 to about 62,000 years), fossil-derived non-renewable matter does not contain significant amounts of radiocarbon, if any at all. See Sparks & Beavan-Athfield, Methodology for Testing the Percentage of Modern Biological Component in Biofuel Blends with Radiocarbon Dating in GNS SCIENCE CONSULTANCY REPORT (2007). Therefore, when compared to a non-renewable carbon, a renewable carbon has a significantly greater probability of being a radiocarbon. As a result, renewable matter can be distinguished from non-renewable matter based on radiocarbon content. Similarly, renewable compounds can be distinguished from non-renewable compounds because the former has a significantly greater proportion of radiocarbon to total carbon count than the latter.

Methods for determining radiocarbon content in a sample include, for example, Liquid Scintillation Counting (LSC), Accelerator Mass Spectrometry (AMS), or Isotope Ratio Mass Spectrometry (IRMS), which are known to those of skill in the art. See, for example, Sparks & Beavan-Athfield, Methodology for Testing the Percentage of Modern Biological Component in Biofuel Blends with Radiocarbon Dating in GNS SCIENCE CONSULTANCY REPORT (2007); Dijs et al., Quantitative Determination by ¹⁴C Analysis of the Biological Component in Fuels, Radiocarbon 48, 315-323 (2006); and Reddy et al., Determination of Biodiesel Blending Percentages Using Natural Abundance Radiocarbon Analysis: Testing the Accuracy of Retail Biodiesel Blends, Environmental Science & Technology 42: 2476-2482 (2008).

Thus, the renewable carbon contents of compounds containing renewable and non-renewable carbons can be determined using LSC, AMS or IRMS. More specifically, the renewable carbon contents of a sample can be determined by measuring the proportion of radiocarbons in the sample to total carbons (i.e. per total carbon count). The proportion of radiocarbon to total carbon count can be compared with that of a standard sample having a renewable carbon content of 100%. It can also be compared with a series of standard samples having known renewable carbon contents that are less than 100%. In addition, the proportion of radiocarbon to total carbon count in non-renewable matter can be used as a base line, i.e. representing a renewable carbon content of 0%. By comparing the proportion of radiocarbon to total carbon count of an unknown sample with that of one or more standards having known renewable carbon content(s), the renewable carbon content of the sample can be determined.

Accordingly, the amount of radiocarbon per total carbon count determined by LSC, AMS or IRMS for non-renewable benzene is insignificant, if detectable at all, as non-renewable benzene is from fossil matter that is millions of years old. As such, this amount can be used as a base line representing 0% renewable carbon content. In contrast, the proportion of radiocarbon to total carbon count determined for renewable limonene extracted from orange peels is significantly greater than the base line, i.e. that of non-renewable benzene.

Furthermore, renewable matters that have renewable carbon contents of 100% (i.e. all the carbons are renewable carbons) such as purified limonene from citrus peels, purified cymene from basil, essential oils extracted from citrus rinds, and γ-terpinene from Melaleuca alternifolia, have a similar proportion of radiocarbon to total carbon count (or similar radiocarbon to stable carbon ratios) if they are from the same time period. Renewable matters are from the same time period if the radiocarbon contents in the atmospheres at the time the matters were grown or biosynthesized are the same. Therefore, compounds having renewable carbon contents of 100% have a characteristic proportion of radiocarbon to total carbon count that reflects atmospheric proportions at the time they are biosynthesized.

II. Cymene from Natural Sources

Cymene is an aromatic hydrocarbon that exists in three isomeric forms: m-cymene, o-cymene and ρ-cymene respectively. P-cymene is naturally-occurring and can be isolated in numerous natural sources as known to those of skill in the art. For example, the most abundant compound in cumin oil is p-cymene at 27.92%. See Park et al., Toxicity of Plant Essential Oils and Their Components against Lycoriella ingenua (Diptera: Sciaridae), Journal of Economic Entomology 101:139-144 (2008). Asllani et al., found that as much as 44% of Albanian thyme oil obtained by steam distillation of air-dried plant material is ρ-cymene. See Asllani et al., Chemical composition of Albanian thyme oil (Thymus vulgaris L.), Journal of Essential Oil Research May/June 2003. Additional non-limiting examples of sources from which renewable ρ-cymene can be isolated include: (1) common thyme Thymus vulgaris (Letchamo & Kireeva, Development of Thymus vulgaris Varieties for North American Commercial Organic Cultivation, HortScience 33: 482 (1998)); (2) ripe blackberry fruits (Perkins-Veazie et al., Changes in Sugars and Volatiles of Ripening Erect Blackberry Fruit, HortScience 32: 434 (1997)); (3) basil (Koba, Antifungal Activity of the Essential Oils from Ocimum gratissimum L. Grown in Togo, J. Sci. Res. 1: 164-71 (2009)); (4) Rosemary, i.e. Rosmarinus officinalis (Tawfik & Read, In Vitro Selection for High Essential Oil Yield in Rosmarinus officinalis, HortScience 26:756 (1991)); (5) Brazillian basil (Vieira & Simon, Chemical Characterization of Basil (Ocimum spp.) Germplasm from Brazil, HortScience 32:464 (1997)); (6) grapefruit (Dou, Volatile Differences of Pitted and Non-pitted ‘Fangio’ Tangerine and White ‘Marsh’ Grapefruit, HortScience 38: 1408 (2003); (7) fennel (Bowes & Zheljazkov, Essential Oil Yields and Quality of Fennel Grown in Nova Scotia, HortScience 39: 1640-43 (2004)); (8) the flowering plant Heliotropium arborescens L. ‘Marine’ (Kays et al., Volatile Floral Chemistry of Heliotropium arborescens L. ‘Marine’, HortScience 40:1237 (2005)); (9) the bush Lippia sidoides (Fontenelle et al., Chemical Composition, Toxicological Aspects and Antifungal Activity of Essential Oil from Lippia sidoides Cham., J. Antimicrob Chemother 59: 934 (2007); (10) the seeds of cumin, i.e. Cuminum cyminum L. (Jirovetz et al., Composition, Quality Control and Antimicrobial Activity of the Essential Oil of Cumin (Cuminum cyminum L.) Seeds from Bulgaria that had been Stored for up to 36 Years, Int'l J. of Food Sci & Tech 40:305 (2005)); (11) the mandarin Citrus unshiu Marc. (Kekelidze et al., Analysis of Terpene Variation in Leaves and Fruits of Citrus unshiu Marc. During Ontogenesis, Flavour and Fragrance Journal 4: 37-41 (2006)); (12) the oils of Greek oregano Origanum vulgare ssp. hirtum (Kokkini et al., Essential Oil Composition of Greek (Origanum vulgare ssp. hirtum) and Turkish (O. onites) Oregano: a Tool for Their Distinction, Journal of Essential Oil Research, July/August (2004)); and (13) the essential oils of the mint Origanum vulgare L (Gurudatt et al., Changes in the Essential Oil Content and Composition of Origanum vulgare L. during Annual Growth from Kumaon Himalaya, Current Science 98: 1010-12 (2010).

Methods of isolating cymene are known in the art. Non-limiting examples include liquid-liquid extraction using a water-insoluble organic solvent such as ethanol, steam distillation and the use of pressurized CO₂. See Asllani et al., Chemical composition of Albanian thyme oil (Thymus vulgaris L.), Journal of Essential Oil Research May/June 2003; Letchamo & Kireeva, Development of Thymus vulgaris Varieties for North American Commercial Organic Cultivation, HortScience 33: 482 (1998); see also Sousa et al., Brazilian J of Chem Eng 19: 229-241 (2002). In general, cymene can be found in the essential oils of the producer organism often along with other lipophilic hydrocarbons such as monoterpenes and monoterpenoids. Thus, cymene-containing oils can be recovered from biological sources using methods employed for the isolation of monoterpenes and monoterpenoids as discussed below. Once recovered, the components in the oils can be further separated using preparative gas chromatography as described in Romanenko & Tkachev, Identification by GC-MS of Cymene Isomers and 3,7,7-Trimethylcyclohepta-1,3,5-Triene in Essential Oils, Chemistry of Natural Compounds, 42:699-701 (2006).

III. Cymene from Cyclic Monoterpene/Monoterpenoid

Renewable cymene also can be obtained by desaturating a cyclic monoterpene.

A. Cyclic Monoterpenes

Cyclic monoterpenes are lipophilic ten-carbon (C₁₀) compounds commonly found in plant resins and essential oils. Cyclic monoterpenes are biosynthesized by an enzyme-catalyzed condensation of isopentenyl pyrophosphate (IPP) and its isomer dimethylallyl pyrophosphate (DMAPP) to form the C₁₀ geranyl-pyrophosphate (GPP).

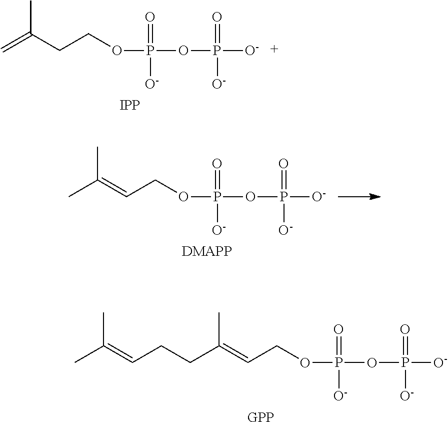

The linear GPP is then cyclicized to a variety of mono and bicyclic C₁₀ compounds, i.e. cyclic monoterpenes. Non-limiting examples of cyclic monoterpenes include: limonene, terpinenes, phellandrenes and terpinolene. Monoterpenes can be modified in numerous ways, for example, by oxidation, rearrangement of the carbon skeleton, or aromatization to form a variety of related compounds including ρ-cymene.

Any cyclic monoterpenes that can be aromatized to ρ-cymene can be used to practice the invention. Cyclic monoterpenes that are particularly useful for generating renewable ρ-cymene have a 5- or 6-member alkane or alkene ring structures. Limonenes, terpinenes, phellandrenes and terpinolene are non-limiting examples of cyclic monoterpenes that can be converted to ρ-cymene, for example, by hydroxylation and dehydration reactions. The following table provides non-limiting examples of cyclic monoterpenes that can be converted to renewable ρ-cymene.

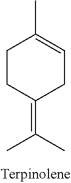

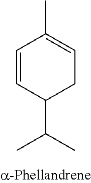

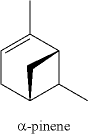

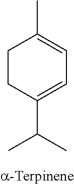

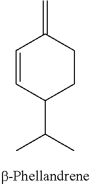

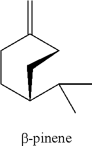

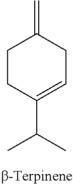

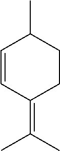

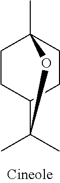

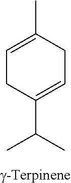

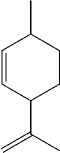

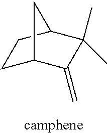

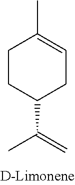

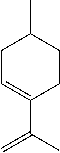

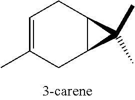

B. Cyclic Monoterpenes from Natural Sources

Cyclic monoterpenes can be isolated from a variety of renewable sources including, without limitation, bacteria, fungi, algae, plants, insects, as well as higher animals such as alligators and beavers. See Wise & Croteau, Monoterpene Biosynthesis in COMPREHENSIVE NATURAL PRODUCT CHEMISTRY: ISOPRENOID BIOSYNTHESIS, pp. 97-153. Cane ed., London: Elsevier (1998). Poulose & Croteau found that the major components of the volatile oil of thyme (Thymus vulgaris L.) are the aromatic monoterpenes, thymol (38%) and p-cymene (23%), and the cyclic diene, γ-terpinene (28%). See Poulose & Croteau, Biosynthesis of Aromatic Monoterpenes, Archives of Biochemistry and Biophysics 187: 307-314 (1978). Letchamo & Kireeva found that the essential oils of various thyme cultivars contain p-cymene (10.87-22.89%), γ-terpinene (2.21-8.85%), and α-pinene (0.87-2.23%). See Letchamo & Kireeva, Development of Thymus vulgaris Varieties for North American Commercial Organic Cultivation, HortScience 33: 482 (1998). In addition, the essential oils of Rosmarinus officinalis contain α-pinene, camphene, β-pinene, cymene, cineol, limonene, linalool, camphor, borneol and bornyl acetate. See Tawfik & Read, In Vitro Selection for High Essential Oil Yield in Rosmarinus officinalis, HortScience 26: 756 (1991). The volatile oils of various species of Brazilian basil (Ocimum species) contain high levels of eugenol (40% to 66%), thymol (33%) or ρ-cymene (28% to 42%). See Vieira & Simon, Chemical Characterization of Basil (Ocimum spp.) Germplasm from Brazil, HortScience 32: 464 (1997). The essential oils of fennel (Foeniculum vulgare Mill.) contain methyl chavicol, fenchone, α-phellandrene, α-pinene, ortho cymene, β-phellandrene, fenchyl acetate, β-pinene, and apiole. See Bowes & Zheljazkov, Essential Oil Yields and Quality of Fennel Grown in Nova Scotia, HortScience 39: 1640-1643 (2004). The main constituents of Lippia sidoides essential oil are thymol (59.65%), E-caryophyllene (10.60%) and ρ-cymene (9.08%). See Fontenelle et al., Chemical Composition, Toxicological Aspects and Antifungal Activity of Essential Oil from Lippia sidoides Cham., J. Antimicrob Chemother 59:934-40 (2007). The essential oil of seeds of cumin (Cuminum cyminum L.) from Bulgaria contains cumin aldehyde (36%), β-pinene (19.3%), ρ-cymene (18.4%) and γ-terpinene (15.3%). See Jirovetz et al., Composition, Quality Control and Antimicrobial Activity of the Essential Oil of Cumin (Cuminum cyminum L.) Seeds from Bulgaria that had been Stored for up to 36 Years, International Journal of Food Science & Technology 40: 305-10 (2005). The most abundant compounds in cumin oil included ρ-cymene (27.92%) followed by γ-terpinene (23.57%), cuminaldehyde (19.08%), β-pinene (11.26%), transanethole. See Park et al., Toxicity of Plant Essential Oils and Their Components against Lycoriella ingenua (Diptera: Sciaridae), Journal of Economic Entomology 101: 139-144 (2008). Essential oils of Greek (Origanum vulgare ssp. hirtum) and Turkish (O. onites) Oregano include sabinene, myrcene, γ-terpinene, borneol and carvacrol, and ρ-cymene. See Kokkini et al., Essential Oil Composition of Greek (Origanum vulgare ssp. hirtum) and Turkish (O. onites) Oregano: a Tool for Their Distinction, Journal of Essential Oil Research July/August 2004.

In addition, blackberry fruit contains α-pinene, eugenol, limonene, p-cymene, α-terpinol, and gernaylacetone. See Perkins-Veazie et al., Changes in Sugars and Volatiles of Ripening Erect Blackberry Fruit, HortScience 32: 434 (1997). Grapefruit contains various amounts of camphene, ethyl hexanoate, α-phellandrene, 3-carene, α-terpinene, ρ-cymene, and limonene. See Dou, Volatile Differences of Pitted and Non-pitted ‘Fangio’ Tangerine and White ‘Marsh’ Grapefruit, HortScience 38: 1408-1409 (2003). The leaves and fruits of a mandarin (Citrus unshiu Marc.) contain limonene, linalol, p-cymene, β-elemene. See Kekelidze et al., Analysis of Terpene Variation in Leaves and Fruits of Citrus unshiu Marc. during Ontogenesis, Flavor and Fragrance Journal 4:37-41 (2006). The flower Heliotropium arborescens L. produces a variety of volatile terpenes including camphene, ρ-cymene, δ-3-carene, α-humulene, δ-1-limonene, linalool, (E)-β-ocimene, α-pinene, and β-thujone. See Kays et al., Volatile Floral Chemistry of Heliotropium arborescens L. ‘Marine’, HortScience 40: 1237-1238 (2005).

In addition, (4R)- and (4S)-limonene can be found in Citrus oils, conifer turpentines, and the essential oils of other plant genera (Guenther B., THE ESSENTIAL OILS Vol. 2, pp. 22-27, R. E. Kreiger, Huntington, N.Y. (1975)), as well as spearmint oil (Wise & Croteau, Monoterpene Biosynthesis in COMPREHENSIVE NATURAL PRODUCT CHEMISTRY: ISOPRENOID BIOSYNTHESIS, pp. 97-153, 147, Cane ed., London: Elsevier 1998. α-Phellandrene can be isolated from Eucalyptus radiata and Eucalyptus dives (see Jacobs & Pickard, Plants of New South Wales (1981) and Boland., Eucalyptus Leaf Oils (1991), respectively). β-phellandrene can be isolated from the oils of water fennel, Canada balsam, and lodgepole pine (Savage et al., Monoterpene Synthases of Pinus contorta and Related Conifers, J. of Biol. Chem. 269:4012-4020 (1994). α-Terpinene can be isolated from the oils of cardamom and marjoram, while γ-terpinene can be isolated from the essential oils of basil, in particular, Ocimum gratissimum by steam distillation. See Koba, Antifungal Activity of the Essential Oils from Ocimum gratissimum L. Grown in Togo, J. Sci. Res. 1: 164-71 (2009). Cymene, in particular, ρ-cymene can be isolated from the essential oils of cumin, thyme as well as basil (Koba, J. Sci. Res. 1: 164-71 (2009)). Thus, monoterpenes can be isolated from the essential oils and terpentines of a large variety of plants and trees, including citrus plants such as oranges, lemons and limes, as well as herbs such as peppermint, spearmint and other aromatic plants.

Methods for isolating cyclic monoterpenes are known to those of skill in the art. For example, cyclic monoterpenes can be isolated from citric fruit rinds using methods known to those of skill in the art including, for example, steam distillation and liquid-liquid extraction using a water-insoluble organic solvent such as methylene chloride, ether, pentane or ethanol. For example, finely grated citric fruit rinds (flavedo or coloured portion of the peel) can be extracted with pentane and then dried over anhydrous sodium sulfate. The sodium sulfate can be removed by filtration, while the solvent can be removed by warming under a low heat and a gentle stream of nitrogen. See above citations for additional examples of methods that can be used to isolate cyclic monoterpenes from numerous renewable sources.

C. Cyclic Monoterpenes from Recombinant Organism

Cyclic monoterpenes can also be biosynthesized using recombinant organisms. Any organism, prokaryotic or eukaryotic, that is amendable to genetic manipulation by recombinant nucleic acid technologies can be used to produce cyclic monoterpenes. Non-limiting examples of organisms that can be used to produce cyclic monoterpenes include microorganisms such as a yeast, other fungi or bacteria. Examples of yeasts that can be used as a producer organism include, without limitation, Saccharomyces cerevisiae, Saccharomyces fermentati, Yarrowia lipolytica, Schizosaccharomyces pombe, Ambrosiozyma monospora, Torulaspora delbrueckii, Phaffia rhodozyma, Kluyveromyces lactis, Pichia pastoris and Hansenula polymorpha. Other fungi that can be used to as producer organisms include, without limitation, filamentous fungi such as Neurospora crassa, Phycomyces blakesleeana or Blakeslea trispora; Ceratocystis moniliformis, Trametes ordorata, Phellinus species (see Halim & Collins 1971; Collins & Halim, Can. J. Microbiol. 18, 56-66 or J. Agric. Food Chem. 20:437-38 (1972)), Aspergillus niger, and Trichoderma reesei. Examples of bacteria that can be used as a producer organism include, without limitation, Escherichia coli, Bacillus subtilis and Streptomyces coelicolor.

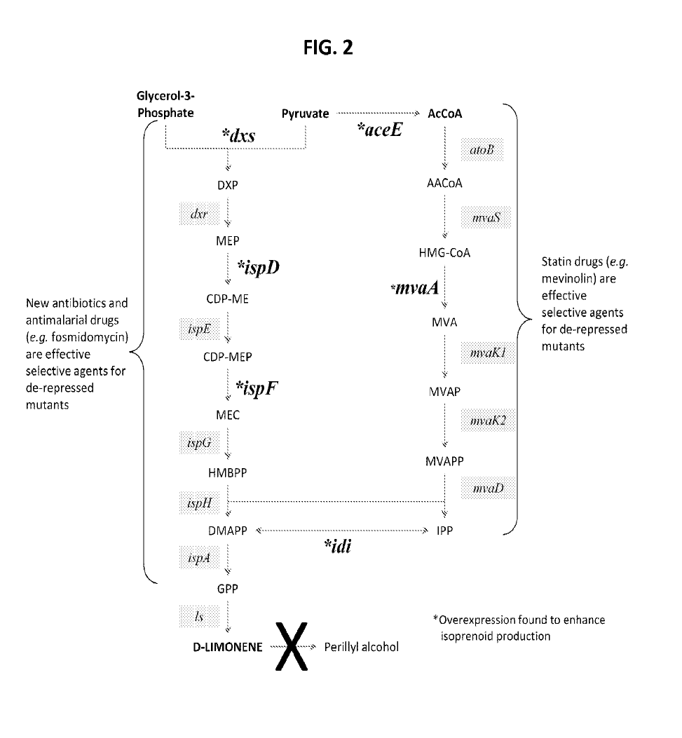

Cyclic monoterpenes are formed from the condensation of two activated isoprene units, in particular, isopentenyl pyrophosphate (IPP) and its isomer dimethylallyl pyrophosphate (DMAPP), to produce the C₁₀ geranyl pyrophosphate (GPP). This step is catalyzed by GPP synthase. GPP is then converted to one or more cyclic monoterpenes through a sequence of reactions catalyzed by a monoterpene cyclase. Thus, the biosynthesis of cyclic monoterpenes involves: (1) IPP and DMAPP biosynthetic enzymes, (2) a GPP synthase and (3) a monoterpene cyclase. IPP and DMAPP can be biosynthesized by two different biosynthetic pathways, the mevalonic acid (MVA) pathway and the non-mevalonic acid (non-MVA) pathway. All higher eukaryotic organisms (including yeasts and fungi) and some bacteria (in particular, Gram positive cocci) produce DMAPP and IPP using the MVA pathway (i.e., HMG CoA Reductase pathway). Many bacteria, protozoa or plant plastids produce DMAPP and IPP using the 2-C-methyl-D-erythritol 4-phosphate/1-deoxy-D-xylulose 5-phosphate (MEP/DOXP) pathway, i.e. non-MVA pathway. Either pathway can be used. The DOXP and MVA pathways are illustrated in FIG. 2.

In the HMG CoA Reductase (MVA) pathway, the enzyme acetyl-CoA acetyltransferase (EC 2.3.1.9) catalyzes the condensation of acetyl-CoA (from the citric acid cycle) with another acetyl-CoA subunit to form Acetoacetyl-CoA. Second, an HMG-CoA synthase (EC 4.1.3.5) catalyzes the condensation of another unit of acetyl-CoA with acetoacetyl-CoA to form 3-hydroxy-3-methylglutaryl-CoA (HMG-CoA). Third, HMG-CoA is reduced to mevalonate by NADPH. This reaction, shown below, occurs in the cytosol and is catalyzed by the enzyme HMG-CoA reductase (mvaA in FIG. 2) (EC 1.1.1.34). Fourth, mevalonate is then converted to 5-phosphomevalonate by mevalonate kinase (EC 2.7.1.36). Fifth, 5-phosphomevalonate is converted to 5-pyrophosphomevalonate by phosphomevalonate kinase (EC 2.7.4.2) as shown below. Sixth, mevalonate-5-pyrophosphate is converted to 3-isopentenyl pyrophosphate (IPP) by mevalonate-5-pyrophosphate decarboxylase (EC 4.1.1.33). Seventh, the enzyme isopentenyl pyrophosphate isomerase (idi in FIG. 2) (EC 5.3.3.2) catalyzes the isomerization of IPP to dimethylallyl pyrophosphate (DMPP).

In the MEP/DOXP (non-MVA) pathway, 1-deoxy-D-xylulose 5-phoshate (DOXP) synthase (dxs in FIG. 2) (EC 2.2.1.7) catalyzes the thiamin diphosphate-dependent condensation of pyruvate and glyceraldehydes-3-phosphate to form DOXP. See P.N.A.S. U.S.A 94: 12857-62 (1997). DOXP synthase is also known as 1-deoxy-D-xylulose-5-phosphatase, deoxyxylulose-5-phosphate synthase, and DXP synthase. Then DOXP reductase (EC 1.1.1.267) acts in the presence of NAD(P)H and a divalent ion such as Mn(II) or Co(II) to convert DOXP to 2-C-methylerythritol 4-phosphate (MEP). See P.N.A.S. U.S.A. 95:9879-84 (1998). DOXP reductase is also known as 1-deoxy-D-xylulose 5-phosphate reductoisomerase, 2C-methyl-D-erythritol 4-phosphate synthase, deoxyxylulose 5-phosphate reductoisomerase, DOXP reductoisomerase, DXP reductoisomerase, Dxr, IspC and yaeM. Third, the enzyme 4-diphosphocytidyl-2-C-methyl-D-erythritol synthase (ispD in FIG. 2) (EC 2.7.7.60) converts MEP to 4-diphosphocytidyl-2-C-methylerythritol (CDP-ME). See Nature Structural Biology 8:641-8 (2001); Tetrahedron Lett 41:703-6 (2000) & P.N.A.S. U.S.A. 96:11758-63 (1999). This enzyme is also known as 2-C-methyl-D-erythritol 4-phosphate cytidylyltransferase, MEP cytidylyltransferase, CDP-ME synthetase, IspD and YgbP. Fourth, 4-diphosphocytidyl-2-C-methyl-D-erythritol kinase (EC 2.7.1.148) converts CDP-ME to 4-diphosphocytidyl-2-C-methyl-D-erythritol 2-phosphate (CDP-MEP). See PNAS USA 97:1062-7 (2000) and Tetrahedron Lett. 41:2925-2928 (2000). This enzyme is also known as 4-(cytidine 5′-diphospho)-2-C-methyl-D-erythritol kinase), 4-(cytidine 5′-diphospho)-2-C-methyl-D-erythritol 2-phosphotransferase, CDP-ME kinase, YchB and IspE. Fifth, the enzyme 2-C-methyl-D-erythritol 2,4-cyclodiphosphate synthase (ispF in FIG. 2) (EC 4.6.1.12) converts CDP-MEP to 2-C-methyl-D-erythritol 2,4-cyclopyrophosphate (MEcPP). See P.N.A.S. U.S.A. 97:2486-90 (2000) and Tetrahedron Lett. 41:3395-3398 (2000). This enzyme is also known as 2-phospho-4-(cytidine 5′-diphospho)-2-C-methyl-D-erythritol CMP-lyase, MECDP-synthase, YgbB, and IspF. Sixth, the enzyme HMB-PP synthase (EC 1.17.4.3) converts MEcPP to (E)-4-Hydroxy-3-methyl-but-2-enyl pyrophosphate (HMB-PP). See P.N.A.S. U.S.A. 98:14837-42 (2001). This enzyme is also known as (E)-4-hydroxy-3-methylbut-2-en-1-yl-diphosphate:protein-disulfide oxidoreductase, GcpE and IspG. Seventh, HMB-PP reductase (EC 1.17.1.2) reduces HMB-PP to IPP and DMAPP. This enzyme is also known as LytB and IspH.

Regardless of the organism selected to produce the cyclic monoterpene, the cyclic monoterpene biosynthetic enzymes (i.e., the genes encoding IPP and DMAPP biosynthetic enzymes, GPP synthase and the cyclic monoterpene cyclase) can be from any organism so long as the nucleic acid coding sequence can be expressed in the producer organism. One, more than one, or all of the biosynthetic enzymes for producing a selected monoterpene can be from the producer organism or from an organism that is phylogenetically close to the producer organism. The enzyme(s) selected can be derived from an organism of the same strain, species, genus or family as the producer organism. For example, if the producer organism is Saccharomyces cerevisiae, then the enzyme(s) selected can be from (1) any yeast (e.g. Candida oleophila, Pichia pastoris or Kluyveromyces lactis), (2) another Saccharomyces (e.g. Saccharomyces uvarum), (3) another Saccharomyces cerevisiae strain or (4) the Saccharomyces cerevisiae strain that has been selected to be producer organism. Thus, the enzyme(s) can be native to the producer organism, i.e. they are not encoded by heterologous nucleic acid sequences that have been introduced into the producer organism from an organism of a different species, or genus altogether. The enzyme(s) can be encoded by and expressed from heterologous nucleic acid sequences derived from one or more organisms of a different strain, species or genus altogether.

Thus, to produce DMAPP and IPP using the HMG CoA Reductase (MVA) pathway, any acetyl-CoA transferase can be used to form Acetoacetyl-CoA. Non-limiting examples of acetyl-CoA transferase that can be used to practice the invention include the enzyme encoded by the atoB gene of Escherichia coli (Gen Bank Accession Number: NP_{—}416728.1) and locus BMD_{—}4393 of Bacillus megaterium (GenBank Accession Number: NC_{—}014103.1). Any H MG-CoA synthase can be used in the second step to produce HMG-CoA. Non-limiting examples of HMG-CoA synthase that can be used to practice the invention include the enzyme encoded by the ERG13 gene of Saccharomyces cerevisiae (GenBank Accession Number: NM_{—}001182489.1) or the mvaS gene from Staphylococcus epidermidis (Gen Bank Accession Number AAG02433.1). Any HMG-CoA reductase can be used in the third step to produce mevalonate. Non-limiting examples of HMG-CoA reductase that can be used to practice the invention include the enzyme encoded by the hmg1 gene of Arabidopsis thaliana (NCBI reference sequence: NM_{—}106299.3) and the hmg1 gene of Saccharomyces cerevisiae (NCBI reference sequence: NM_{—}001182434.1). Any mevalonate kinase can be used in the fourth step to produce 5-phosphomevalonate. Non-limiting examples of mevalonate kinase that can be used to practice the invention include the enzyme encoded by the erg12 gene of Saccharomyces cerevisiae (GenBank Accession Number: EDN64144.1) and the mvaK1 gene of Staphylococcus aureus (NCBI reference sequence: YP_{—}001315773.1). Any phosphomevalonate kinase can be used in the fifth step to produce 5-pyrophosphomevalonate. Non-limiting examples of phosphomevalonate kinase that can be used to practice the invention include the enzyme encoded by the ERGS gene of Saccharomyces cerevisiae (GenBank accession number: AAA34596.1) and phosphomevalonate kinase of Staphylococcus aureus JH1 (NCBI reference sequence YP_{—}001315775.1). Any mevalonate-5-pyrophosphate decarboxylase (also known as diphosphomevalonate decarboxylase) can be used in the sixth step to produce IPP. Non-limiting examples of mevalonate-5-pyrophosphate decarboxylase that can be used to practice the invention include the enzyme encoded by the mvd1 gene of Saccharomyces cerevisiae (NCBI reference sequence: NM_{—}001183220.1) and locus YALIOF05632g of Yarrowia lipolytica (NCBI reference sequence XM_{—}505041.1). Any isopentenyl pyrophosphate isomerase can be used to catalyze the isomerization of IPP to DMAPP. Non-limiting examples of isopentenyl pyrophosphate isomerase that can be used to practice the invention include the enzyme encoded by the idi1 gene of Saccharomyces cerevisiae (NCBI reference sequence NM_{—}001183931.1) and the idi gene of Eschericia coli (NCBI reference sequence: AC_{—}000091.1).

To produce DMAPP and IPP using the MEP/DOXP (non-MVA) pathway, any DOXP synthase can be used in the first step to produce DOXP. Non-limiting examples of DOXP synthases that can be used to practice the invention include the enzyme encoded by the dxs gene of Escherichia coli (NCBI reference sequence YP_{—}002396495.1) and dxps1 of Arabidopsis thaliana (NCBI reference sequence NM_{—}180289.3). Any DOXP reductase can be used in the second step to produce MEP. Non-limiting examples of DOXP reductase that can be used to practice the invention include ispC of Escherichia coli (NCBI reference seq NC_{—}000913.2) and dxr of Arabidopsis thaliana (NCBI reference sequence: NM_{—}125674.2). Any 4-diphosphocytidyl-2-C-methyl-D-erythritol synthase can be used in the third step to produce CDP-ME. Non-limiting examples of 4-diphosphocytidyl-2-C-methyl-D-erythritol synthase that can be used to practice the invention include ispD of Escherichia coli (Gen Bank Accession Number: 000096.2) and ispD of Arabidopsis thaliana (NCBI reference sequence: NM_{—}126305.2). Any 4-diphosphocytidyl-2-C-methyl-D-erythritol kinase can be used in the fourth step to produce CDP-MEP. Non-limiting examples of 4-diphosphocytidyl-2-C-methyl-D-erythritol kinase that can be used to practice the invention include ispE of Bacillus megaterium (NCBI reference sequence: NC_{—}014103.1) and ATCDPMEK of Arabidopsis thaliana (NCBI reference sequence: NM_{—}128250.3). Any 2-C-methyl-D-erythritol 2,4-cyclodiphosphate synthase can be used in the fifth step to produce MEcPP. Non-limiting examples of 2-C-methyl-D-erythritol 2,4-cyclodiphosphate synthase that can be used to practice the invention include ispF of Arabidopsis thaliana (NCBI reference sequence: NM_{—}180640.20) and ispF of Escherichia coli (NCBI reference sequence: NP_{—}417226.1). Any HMB-PP synthase can be used in the sixth step to produce HMB-PP. Non-limiting examples of HMB-PP synthase that can be used to practice the invention include ispG of Escherichia coli (NCBI reference sequence NP_{—}417010.1) and ispG of Bacillus cereus (NCBI reference sequence: YP_{—}085608.1). Any HMB-PP reductase can be used in the seventh step to produce IPP and DMAPP. Non-limiting examples of HMB-PP reductase that can be used to practice the invention include ispH of Escherichia coli (NCBI reference sequence: YP_{—}001729012.1) and HDR of Arabidopsis thaliana (NCBI reference sequence: NM_{—}119600.3).

From DMAPP and IPP, a cyclic monoterpene can be produced in two enzyme-catalyzed steps. In the first step, geranyl pyrophosphate synthase (GPP synthase; E.C. 2.5.1.1) catalyzes the condensation of IPP and DMAPP to form geranyl pyrophosphate (GPP), a linear C₁₀ compound having the following structure.

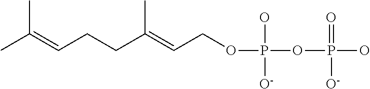

See Ogura & Koyama, Chem Rev 98:1263-76 (1998). Any GPP synthase can be used to condense DMAPP and IPP. Non-limiting examples of GPP synthases that can be used include those isolated from Saccharomyces cerevisiae or Schizosaccharomyces pombe, or a filamentous fungi Phycomyces blakesleeana or Blakeslea trisporia or a plant Arabidopsis thaliana. Non-limiting examples of GPP synthases that can be used to practice the invention include the gps1 of Arabidopsis thaliana (see NCBI accession numbers NM_{—}001036406.2 and NP_{—}001031483.1 for the nucleic acid and protein sequences, respectively) and the gpps of Vitis vinifera (see GenBank: AY351862.1 for the nucleic acid sequence and AAR08151.1 for the polypeptide sequence). An additional example of a GP synthase that can be used to practice the invention is the enzyme from Solanum lycopersicum provided by GenBank Accession numbers DQ286930.1 (nucleotide sequence) and ABB88703.1 (protein sequence).

The linear GPP produced by the GPP synthase-catalyzed condensation of IPP and DMAPP can be converted to one or more cyclic monoterpenes by a monoterpene cyclase. See, for example, Bohlmann et al., J Biol Chem 272:21784-92 (1997). Any monoterpene cyclase can be used to convert a liner GPP to a cyclic monoterpene so long as it produces the cyclic monoterpene that can be desaturated to cymene. Some monoterpene cyclases catalyze the formation of distinct monoterpenes, while others catalyze the formation of multiple monoterpene products. For example, the (−)-4S-limonene synthase of spearmint (Mentha spicata L) catalyzes the formation of 4S-limonene, as well as α- and β-pinenes. See Colby et al., J. Biol. Chem. 268: 23016-24 (1993). The γ-terpinene synthase from thyme (Thymus vulgaris) leaves catalyzes the formation of γ-terpinene, as well as small amounts of α-thujene, myrcene, α-terpinene, limonene, linalool, terpinen-4-ol, and α-terpineol. See Alonso & Croteau, Arch Biochem Biophys 286:511-7 (1991). Non-limiting examples of monoterpene cyclases that can be used to practice the invention include: (1) 4S-limonene synthase, which catalyzes the conversion of GPP to (4S) limonene (see Alonso et al., J. Biol. Chem. 267:7582-87 (1992) & Colby et al., J. Biol. Chem. 268: 23016-24 (1993)); (2) γ-terpinene synthase, which catalyzes the conversion of GPP to γ-terpinene and α-terpinene (see Alonso & Croteau, Arch Biochem Biophys 286:511-7 (1991); see also Poulose & Croteau, Arch Biochem Biophys 191: 400-11 (1978)); (3) phellandrene synthase, which catalyzes the conversion of GPP to β-phellandrene (Thomas et al., J Biol Chem 269:4012-20 (1994)). Sequences of non-limiting examples of monoterpene cyclases that can be used to practice the invention include those provided in: (1) GenBank AF051901.1 (nucleotide) and AAC26018.1 (protein) for Salvia officinalis (+)-sabinene synthase; (2) GenBank AF514288 (nucleotide) and AAM53945.1 (protein) for Citrus limon (−)-β-pinene synthase; (3) GenBank AF543530.1 (nucleotide) and AAO61228.1 (protein) for the Pinus taeda α-pinene synthase; (4) GenBank AF139206.1 (nucleotide) and AAF61454.1 (protein) for Abies grandis terpinolene synthase (agc9); (5) GenBank AB110640.1 (nucleotide) and BAD27259.1 (protein) for Citrus unshiu γ-terpinene synthase; and (6) GenBank AB110637.1 (nucleotide) and BAD27257.1 (protein) for Citrus unshiu d-limonene synthase.

Any organism, prokaryotic or eukaryotic, that is amendable to genetic manipulation by recombinant nucleic acid technologies can be used to produce cyclic monoterpenes. All the genes involved in the biosynthesis of a selected cyclic monoterpene (i.e. genes encoding the DMAPP/IPP biosynthetic enzymes, the GPP synthase and monoterpene cyclase) can be introduced into a selected organism and expressed using recombinant DNA technologies known to those of skill in the art. See, for example, Sambrook & Russell, MOLECULAR CLONING: A LABORATORY MANUAL, 3rd Edition, Cold Spring Harbor Laboratory Press, Cold Spring Harbor, N.Y. (2001). In addition, since DMAPP and IPP are universal building blocks, virtually all living organisms have IPP and DMAPP biosynthetic enzymes and can produce cyclic monoterpenes if they are engineered to express a GPP synthase and a monoterpene cyclase. Many organisms produce GPP as an intermediate in the production of other secondary metabolites such as isoprenoids of C₁₀ or greater. These organisms can produce a selected monoterpine if they are engineered to express the corresponding monoterpene cyclase. Where all the biosynthetic enzymes for a select monoterpene are present, the level of expression/activity of any one or more enzymes can be modified to enhance production of the cyclic monoterpene. For example, the activity of regulatory genes affecting expression of one or more cyclic monoterpene biosynthetic enzymes can be modified. Furthermore, additional, e.g. exogeneous, copies of nucleic acids encoding IPP and DMAPP biosynthetic enzymes, GPP synthase and/or monoterpene cyclase can be introduced and expressed.

A cyclic monoterpene producer organism can be a fungal or bacterial host that has tolerance to monoterpenes. For example, Pseudomonas putida DSM 12264, which has a tolerance to limonene of 20 g/l (150 mM), can be used. As P. putida possesses the DOXP pathway, the MVA pathway can be cloned and introduced into P. putida to increase carbon flux to the isoprenoid/monoterpene pathway. The MVA pathway, which includes mvaS, idi, mvaA, mvaD, mvaK2 and mvaK1, can be cloned from ATCC 35210D-5 on a single 6.4 Kbp fragment using methods known to those of skill in the art. To increase biosynthesis of a monoterpene such as limonene, the activities of genes in the DOXP and MVA pathways can up regulated (FIG. 2). More specifically, biosynthesis of limonene can be increased by overexpression of the following DOXP and MVA pathway genes in the host producer: aceE (converts pyruvate to AcetylCo-A), mvaA (converts HMG-CoA to mevalonic acid), dxs (convert pyruvate to glycerol-3-phosphate), ispD (converts MEP to CDP-ME), ispF (converts CDP-MEP to MEC) and idi (interconverts DMAPP and IPP). In addition, to increase isoprenoid/monoterpene biosynthesis, one or more genes in the limonene degradative pathway of the producer organism, for example, limonene monooxygenase, can be knocked out. Elimination of the Imo gene activity (converts limonene to perillyl alcohol) can also increase limonene biosynthesis. Methods of down or up regulating gene activity are well known to those of skill in the art. The activity of genes involved in primary metabolism also can be modified in the host organism to increase biosynthesis of limonene. More specifically, glk (converts D-glucose to glucose-6-phosphate), as well as aceE (converts pyruvate to acetyl Co-A), can be over expressed to enhance biosynthesis of limonene. The activities of genes encoding enzymes in the TCA cycle and the glyoxylate shunt also can be down regulated or eliminated to further increase isoprenoid biosynthesis. For example, the activities of glutamate dehydrogenase of the TCA cycle and formate dehydrogenase of the glyoxylate shunt can be down regulated or eliminated to increase limonene biosynthesis.

The recombinant organism can be cultivated under conditions that enhance monoterpene biosynthesis using methods known to those of skill in the art. For example the producer organism can be grown under low nutrient conditions such as low nitrogen or low phosphorus. See, for example, Schmelz et al., Nitrogen Deficiency Increases Volicitin-Induced Volatile Emission, Jasmonic Acid Accumulation, and Ethylene Sensitivity in Maize, Plant Physiology: 133; 295-306 (2003). Cyclic monoterpene can be isolated from the producer organism using methods known to those of skill in the art. The monoterpene-containing oils from the fermentation medium of a recombinant organism can be recovered using a variety of methods including, without limitation, centrifugation or extraction using an organic solvent such as hexane, diethyl ether, ethanol or chloroform. For example, limonene, which has solubility in water of less than 10 mg/L, can be recovered from the fermentation broth as an oil-water emulsion. The emulsion can be pumped into a settling tank for separation of the limonene oil and water phases (FIG. 2). The limonene phase can be siphoned off, while the water and cells can be re-introduced into the fermenter. The fermenter can be outfitted with a drop tank, where the limonene oil/water broth emulsion can be pumped and allowed to settle for a brief period. After sufficient separation of the oil and water, the limonene oil layer can be sent to downstream for processing, e.g. dehydrogenation, while the de-oiled broth can be recycled into the fermenter.

D. Conversion of Cyclic Monoterpene to Cymene

Cyclic monoterpenes can be converted to cymene through one or any combination of isomerization, oxidation, hydroxylation, dehydration, or desaturation reactions known to those of skill in the art. In addition, a cyclic monoterpene can be converted to cymene by dehydrogenation. α-Terpinene, for example, can be converted to cymene by dehydrogenation on sulfated zirconia. See Comelli et al., Isomerization of α-Pinene, Limonene, α-Terpinene and Terpinolene on Sulfated Zirconia, Journal of the American Oil Chemists' Society 82:531-35 (2005). Non-limiting examples of methods for performing dehydrogenation include dehydrogenation by N-lithioethylenediamine, liquid phase dehydrogenation using palladium fixed on charcoal and heteropoly acid oxidative dehydrogenation. Roberge et al., for example, describes the use of carriers impregnated with palladium (Pd) to convert α-pinene and β-pinene to ρ-cymene by dehydrogenation. See Roberge et al., Catalytic Aspects in the Transformation of Pinenes to ρ-Cymene, Applied Catalysis A: General 215: 111-124 (2001). Neumann & Lissel describes the oxidative dehydrogenation of cyclic dienes (e.g. limonene to cymene) using mixed addenda heteropoly acid H₃PMo₁₀V₂O₄₀ as a catalyst. See Neumann & Lissel, Aromatization of hydrocarbons by Oxidative Dehydrogenation Catalyzed by the Mixed Addenda Heteropoly Acid H₃PMo₁₀V₂O₄₀, Journal of Organic Chemistry 54: 4607-10 (1989). Leita et al. describes the hydrogenation of cineole to ρ-cymene. See Leita et al., Production of ρ-Cymene and Hydrogen from a Bio-renewable Feedstock-1,8-Cineole (Eucalyptus Oil), Green Chemistry 12: 70 (2010).

D-limonene extracted from orange peels can be converted to its isomer α-terpenene when warmed with mineral acid or maleic anhydride. And α-terpinene can be converted to the aromatic ρ-cymene by oxidation, for example, auto-oxidation, through use of a metal catalyst or action of a dehydrogenase or oxidase. Buhl et al. describes the conversion of limonene to cymene directly by dehydrogenation catalyzed by palladium impregnated in silica. See Buhl et al., Production of ρ-Cymene from α-Limonene over Silica Supported Pd Catalysts, Applied Catalysis A: General 188:287-299 (1999). Lopes et al. describes the conversion of limonene to cymene in liquid phase using a zeolite catalyst such as a dealuminated HY zeolite. Lopes et al., Aromatization of Limonene with Zeolites Yin NATURAL PRODUCTS IN THE NEW MILLENNIUM: PROSPECTS AND INDUSTRIAL APPLICATIONS 429-36, Kluwer Academic Publishers (2002). Zhao et al. describes the conversion of limonene to cymene using soluble palladium nanoparticle catalysts in aqueous phase. See Zhao et al., Aqueous-phase Biphasic Dehydroaromatization of Bio-derived Limonene into p-Cymene by Soluble Pd Nanocluster Catalysts, Journal of Catalysis 254: 244-50 (2008). Martin-Luengo et al. describes the conversion of limonene to cymene under “solvent-free” conditions over mesoporous silica-alumina supports heated by microwave irradiation. See Martin-Luengo et al., Synthesis of p-Cymene from Limonene, a Renewable Feedstock, Applied Catalysis B: Environmental 81: 218-24 (2008). Romanenko & Tkachev describes dehydrogenation of limonene with H₂SO₄ to produce ρ-cymene. Romanenko & Tkachev, Identification by GC-MS of Cymene Isomers and 3,7,7-Trimethylcyclohepta-1,3,5-Triene in Essential Oils, Chemistry of Natural Compounds 42:699-701 (2006). Romanenko & Tkachev also describes the conversion of 3-carene by dehydrogenation to H₂SO₄ to produce m-cymene and ρ-cymene. See id. Phellandrenes such as α-phellandrene can also be converted to ρ-cymene by dehydrogenation as described by Reggel et al., The Lithium-Ethylenediamine System. II. Isomerization of Olefins and Dehydrogenation of Cyclic Dienes, Journal of Organic Chemistry 23: 1136-39 (1958).

Of the terpinenes, α-terpinene can be converted to ρ-cymene in liquid phase on sulfated zirconia having 15% H₂SO₄ as described by Comelli et al., Isomerization of α-Pinene, Limonene, α-Terpinene, and Terpinolene on Sulfated Zirconia, Journal of the American Oil Chemists' Society 82:531-35 (2005). α-Terpinene can be converted to ρ-cymene in a photocatalytic dehydrogenation reaction with benzophenone and cupric ions under concentrated sunlight irradiation as described by Avcibasi et al., Photochemical Reactions of α-Terpinene and Acenaphthene under Concentrated Sunlight, Turk J Chem 27: 1-7 (2003). In addition, γ-terpinene be aromatized in the presence of: (1) hydrogenation catalysts such as platinum, palladium and nickel (i.e. by reverse hydrogenation); (2) the elements sulfur and selenium, or (3) quinones such as 2,3-Dichloro-5,6-dicyano-1,4-benzoquinone (DDQ), a mild oxidizing agent used as a radical acceptor. γ-Terpinene can be converted to ρ-cymene in solvent-free dehydrogenation reactions as described by Szuppa et al., Solvent-free Dehydrogenation of γ-Terpinene in a Ball Mill: Investigatin of Reaction Parameters, Green Chemistry 12: 1288-94 (2010).

Cyclic monoterpenes such as γ-terpinene can be converted to ρ-cymene using a dehydrogenase enzyme. A dehydrogenase is an enzyme that oxidizes a substrate by transferring one or more hydrides (H−) to an acceptor, usually NAD+/NADP+ or a flavin coenzyme such as FAD or FMN. The dehydrogenase-catalyzed conversion of a cyclic monoterpene to ρ-cymene can occur in vivo (in the host organism) through the action of a native dehydrogenase or a heterologous dehydrogenase that has been introduced into the host cell. Alternatively, the dehydrogenase can act on a purified or partially purified monoterpene sample obtained from a natural, renewable source or from the fermentation of a recombinant microorganism. Various dehydrogenase enzymes can catalyzing this reaction to varying degrees and can be improved upon by applying standard enzyme improvement strategies such as site-directed mutagenesis or directed evolution. An example of a dehydrogenase that can be used in the methods of the invention is the dehydrogenase from Thymus vulgaris described by Poulose & Croteau, Archives of Biochem & Biophys 187: 307-314 (1978). Additional non-limiting examples of dehydrogenases that can be used in the methods of the invention include: (1) Pichia stipitis CBS 6054 NAPDH dehydrogenase (old yellow enzyme) (EPB1), the nucleotide and polypeptide sequences of which are provided in NCBI Reference XM_{—}001385041.1 and XP_{—}001385078.1, respectively; (2) the Debaryomyces hansenii CBS767 DEHA2CO4576p (DEHA2CO4576g) dehydrogenase, the nucleotide and polypeptide sequences of which are provided in NCBI Reference XM_{—}002770122.1 and XP_{—}002770168.1, respectively; (3) the Saccharomyces cerrevisiae S288c Old Yellow Enzyme (OYE2), the nucleotide and polypeptide sequences of which are provided in NCBI Reference NM_{—}001179310.1 and NP_{—}012049.1, respectively; and (4) the Kluyveromyces lactis NRRL Y-1140, KLLA0A09075g dehydrogenase, the nucleotide and polypeptide sequences of which are provided in NCBI Reference XM_{—}451397.1 and XP_{—}451397.1, respectively.

Cymene can be generated using a purified monoterpene or a partially purified monoterpene-containing sample. For example, the feed stock for producing cymene can be a purified sample of a selected monoterpene such as, without limitation, limonene, α-, β- or γ-terpinene, α- or β-phellandrene, α- or β-pinene, camphene or carene. The feed stock for producing cymene can also be a partially purified sample containing one or more monoterpene(s) produced by an organism. In this case, the feedstock can be composed of any one or combinations of limonene, α-, β- or γ-terpinene, α- or β-phellandrene, α- or β-pinene, camphene, careen, as well as any other known monoterpene or monoterpenoid. The feedstock can also include cymene. Methods for the isolation and purification of monoterpenes are known to those of skill in the art including any one more more of the following separation steps: centrifucation, solvent extraction, distillation, drying, crystallization and precipitation.

IV. Renewable Aromatic Compounds of the Invention

The invention provides renewable cumene and renewable toluene, the products of the transalkylation of benzene with ρ-cymene obtained from renewable matter. From renewable cumene and renewable toluene, renewable forms of a variety of related aromatic compounds can be produced. For example, renewable cumene can be converted to renewable phenol and acetone, which can be condensed to produce Bisphenol A (BPA). Renewable toluene can be converted to renewable benzoic acid, as well as toluene diisocyanate, renewable xylenes (meta-, ortho- and para-isoforms), renewable benzene, and renewable methane. Renewable meta-xylene, ortho-xylene and para-xylene can be converted to isophthalic acid, phthalic anhydride and terephthalic acid (TPA), respectively. Renewable benzene can be converted to renewable cyclohexane (and then renewable cyclohexanone), as well as a variety of alkylated benzenes having one or more methyl, isopropyl, or methyl and isopropyl substituents, including, without limitation, renewable toluene, renewable cumene, renewable cymene, and renewable di-isopropyl benzene. Renewable ρ-cymene can be converted to renewable cresol as well as terephthalic acid using methods known to those of skill in the art.

A. Renewable Cumene, Toluene, Benzene & Cymene from Transalkylation of Benzene with Cymene

Renewable cumene, renewable toluene and renewable benzene can be obtained by the transalkylation of benzene with renewable cymene. Renewable or non-renewable benzene can be used to produce renewable cumene and renewable toluene. Non-renewable benzene can be obtained from fossil matter in numerous ways. For example, it can be recovered from pyrolysis gasoline which is a byproduct of steam cracking of lower parafins (e.g. propane) or higher hydrocarbons (e.g. naphtha). Renewable benzene can be obtained using a method of the invention as described herein. The benzene produced using a method of the invention is composed of at least one renewable carbon, and as such, it will have a radiocarbon content that is greater than that of benzene from fossil matter as discussed above.

Cymene, e.g., ρ-cymene can be isolated from renewable matter such as Origanum vulgare (oregano). ρ-Cymene can also be generated by the dehydrogenation of a cyclic monoterpene such as limonene obtained from renewable matter such as citrus rinds or biosynthesized by a recombinant microorganism such as a yeast or bacterium. Methods for obtaining renewable ρ-cymene are known to those of skilled in the art and are also described further in the sections below. All of the carbons in renewable ρ-cymene are renewable carbons because the cymene is either isolated from renewable matter, generated by the dehydrogenation of a cyclic monoterpene obtained from renewable matter, or biosynthesized by a recombinant organism. Thus, the ρ-cymene has a renewable carbon content of 100%

The following scheme illustrates the renewable products that can be obtained from the transalkylation of non-renewable benzene with renewable ρ-cymene.

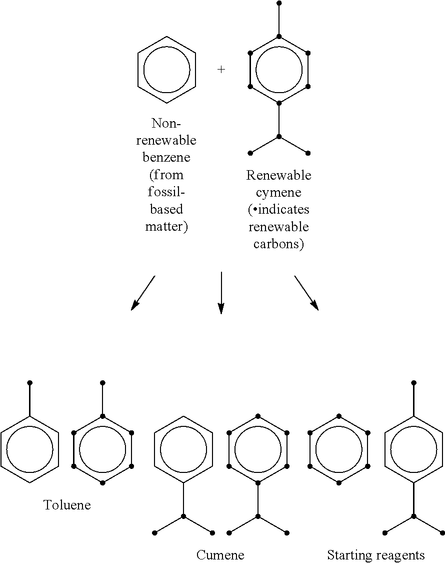

Renewable carbons are marked with bullets (•) to distinguish from non-renewable carbons. Transalkylation of benzene with renewable ρ-cymene can produce two forms of renewable cumene and toluene—the abundance of each the renewable form depends on whether the transalkylation reaction favors migration of the methyl or the isopropyl substituent of ρ-cymene to benzene. In addition, renewable forms of the starting reagents—i.e. benzene and cymene, may also be produced. Thus, the separation of the various products of the transalkylation reaction and any remaining starting reagents would yield compositions having different renewable carbon contents.

More specifically, toluene obtained from the transalkylation reaction illustrated above can be one of two renewable forms, the first form having a renewable carbon content of 14% since only the carbon in the methyl substituent is a renewable carbon derived from ρ-cymene, and the second form having a renewable carbon content of 100% since all the carbons are renewable carbons derived from ρ-cymene. Toluene that is composed of equal amounts of these two forms has a renewable carbon content of about 57% (i.e. 8 of 14 carbons are renewable carbons). Toluene which is composed of different amounts of these two renewable forms can have a renewable carbon content of about 14% to about 100%. Toluene having 100% renewable carbons can be obtained where the transalkylation reaction favors migration of the isopropyl group of cymene to benzene. See Hanai et al., Migration of Alkyl Groups of p-Cymene, Chiba Daigaku Bunrigakubu Kiyo Shizen Kagaku 5: 53-5 (1967). Renewable toluene, or toluene that is a mixture of the two renewable forms, can be distinguished from non-renewable toluene in that the proportion of radiocarbon to total carbon in the former is greater than that of non-renewable toluene alone.

Similarly, cumene obtained from the above transalkylation reaction can be one of two renewable forms: one form having a renewable carbon content about 33% (3 of 9 carbons are renewable carbons) since only the carbons in the isopropyl substituent are renewable carbons derived from ρ-cymene, and the second renewable form having a renewable carbon content of 100% since all the carbons are renewable carbons derived from ρ-cymene. Cumene produced by the transalkylation reaction can have a renewable carbon content of about 33% to about 100%. Cumene that is composed of different amounts of these two renewable forms can have a renewable carbon content of about 33% to about 99% or about 100%. Renewable cumene, or cumene that is a mixture of two renewable forms, can be distinguished from non-renewable cumene in that the proportion of radiocarbon to total carbon in the former is greater than that of non-renewable cumene alone.

The above transalkylation can also generate renewable forms of the starting materials. When both the methyl and isopropyl groups of ρ-cymene are transferred to non-renewable benzene, the products formed are: (1) renewable benzene in which all six carbons are renewable carbons derived from ρ-cymene and (2) renewable cymene, e.g., ρ-cymene, in which four of nine carbons are renewable carbons. Thus, cymene can be of two renewable forms. One renewable form is the original starting reagent, which has a renewable carbon content of 100%. The other renewable form of cymene, e.g. o-, m-, ρ-cymene or a combination thereof, can be produced by the transalkylation reaction. This form has renewable carbons in the methyl and isopropyl substituents that are derived from the original renewable reagent. As such, it has a renewable carbon content about 40% (4 of 10 carbons are renewable carbons). Cymene which is composed of equal amounts of these two renewable oms has a renewable carbon content of about 70% (i.e. 14 of 20 carbons are renewable carbons). And cymene resulting from the transalkylation reaction can have a renewable carbon content of about 40% to about 100%.

B. Renewable Xylene and Renewable Benzene from Toluene Disproportionation

The renewable toluene produced by transalkylation of benzene with cymene can be converted to renewable xylenes by a toluene disproportionation reaction in which the methyl substituent is transferred from one toluene molecule to the other to form benzene and xylenes (meta-, ortho- and para-isomers). Methods for performing disproportionation are known to those of skill in the art. Tsai et al., for example, describes several methods and conditions for performing toluene disproportionation using ZSM-5 catalysts. See Tsai et al., Disproportionation and Transalkylation of Alkylbenzene over Zeolite Catalysts, Applied Catalysis A: General 181:355-98 (1999). The generation of ρ-xylene by toluene disproportionation is shown below.

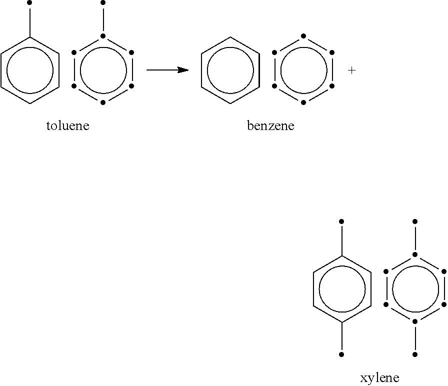

The benzene produced in the toluene disproportionation reaction can be one of two forms: one composed of renewable carbons, and the second composed of non-renewable carbons. Thus, the renewable carbon content of benzene from the toluene disproportionation reaction will reflect the proportion of toluene molecules that have renewable carbons in the phenyl group, and this can be as little as about 1% to as much as about 100%. Benzene having about 100% renewable carbon content can be obtained by the disproportionation of toluene that has about 100% renewable carbon content. Renewable benzene, or benzene that is a mixture of renewable and non-renewable benzenes, can be distinguished from non-renewable benzene in that the proportion of radiocarbon to total carbon in the renewable benzene or the mixture is greater than that of non-renewable benzene alone.

Similarly, the xylene product of the toluene disproportionation reaction can be a mixture of two renewable forms: one in which only the carbons in the methyl substituents are renewable carbons, and the second in which all of the carbons are renewable carbons. The first form has a renewable carbon content of 25% (2 of 8 carbons are renewable carbons), while the second form has a renewable carbon content of 100%. Thus, the xylene produced by toluene disproportionation can have a renewable carbon content from about 25% to about 100%. Xylene with about 100% renewable carbon content can be obtained from a toluene feedstock that has about 100% renewable carbon content. Renewable xylene can be distinguished from non-renewable xylene in that the proportion of radiocarbon to total carbon in the former is greater than that of non-renewable cumene alone.

C. Renewable Benzene from Hydrodealkylation

Renewable toluene produced by transalkylation of benzene with cymene can also be used as a feedstock to produce renewable benzene and renewable methane using conventional hydrodealkylation. For example, renewable toluene can be mixed with H₂, and the mixture can be passed over a chromium, molybdenum, or platinum oxide catalyst at about 500° C. to about 600° C. and about 40 atmospheric pressures to about 60 atmospheric pressures. Alternatively, higher temperatures can be used in place of a catalyst. Under these conditions, renewable toluene is dealkylated to produce benzene and methane as shown below.

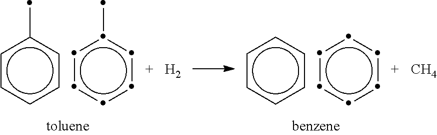

(Renewable cumene can be converted to renewable benzene and propane in a similar hydrodealkylation process as shown below.) The renewable carbon content of benzene from the toluene hydrodealkylation reaction will reflect the proportion of toluene molecules that have renewable carbons in the phenyl group, and as discussed above, this can be as little as about 1% to as much as about 100%. Benzene with about 100% renewable carbon content can be obtained by the hydrodealkylation of toluene that has about 100% renewable carbon content. Methods for performing hydrodealkylation are known to those of skill in the art. For example, see Golubyatnikov et al., Hydrodealkylation of Toluene Using Hydrogen-rich Reformer Gas, Chemistry and Technology of Fuels and Oils 23:418-20 (1987); Grenoble, The chemistry and Catalysis of the Toluene Hydrodealkylation Reaction: I. The Specific Activities and Selectivities of Group VIIB and Group VIII Metals Supported on Alumina, Journal of Catalysis 56:32-39 (1979); Shull & Hixson, Kinetics of Thermal Hydrodealkylation of Mesitylene, m-Xylene and Toluene, Ind Eng Chem Process Des Dev 5:146-50 (1966); and Al-Khowaiter, Hydrodealkylation of Toluene Using Supported Nickel Catalysts: Effect of Molybdenum on Activity and Selectivity, J King Saud Univ, Vol. 8, Science (2), pp. 207-21 (A.H. 1416/1996). Toluene can be hydrodealkylated under catalytic or thermal conditions. Toluene (as well as heavier aromatics that may be present) can be heated with a gas containing hydrogen at a specific selected pressure. The stream is moved past a dealkylation catalyst in the reactor where the toluene (and other aromatics) reacts with hydrogen to generate benzene and methane. Benzene can be separated from methane using a separator operating at high pressure. After removal of the methane, the product is sent to a fractionalization column where it is distilled to recover benzene. Any unreacted material is recycled to the feed.

D. Renewable Phenol and Bisphenol-A from Renewable Benzene or Renewable Cumene

Renewable benzene or renewable cumene, produced as described above, can be used as feedstock to produce renewable phenol. Phenol is produced from benzene and propylene in a process that includes cumene as an intermediate, i.e. the cumene process. The overall chemical reaction for the cumene process is as follows.

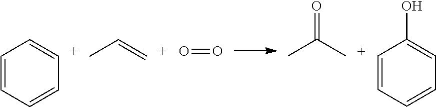

Step I of the cumene process is the Friedel-Crafts alkylation of benzene with propylene in the presence of a catalyst such as phosphoric acid to produce cumene (I) as shown below.

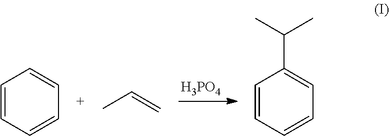

In step III, a cumene radical (II) is formed as benzylic hydrogen is removed by oxidation in air.

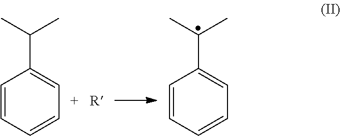

In step III, the cumene radical reacts with O₂ to produce cumene hydroperoxide radical (III).

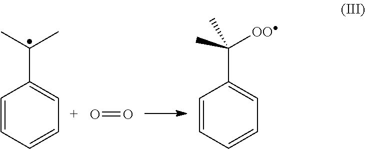

In step IV, the cumene hydroperoxide radical abstracts benzylic hydrogen from another cumene molecule to form cumene hydroperoxide and second cumene radical.

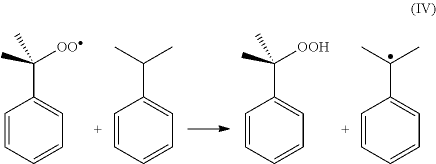

In step V, the cumene radical reacts with oxygen to form more cumene hydroperoxides, which can be hydrolyzed in an acidic medium to give phenol and acetone.

Step V:

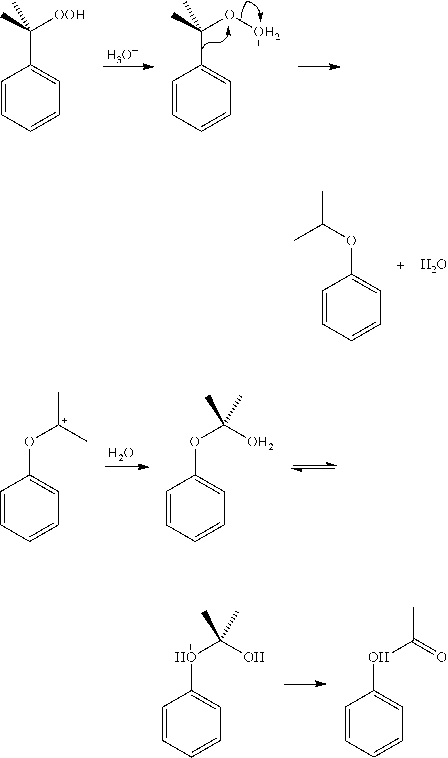

The resulting phenol and acetone can be extracted by distillation. The cumene process for producing phenol is well known in the art. See, for example, Zakoshansky, The Cumene Process for Phenol-Acetone Production, Petroleum Chemistry 47:273-84 (2007); see also Luyben, Design and Control of the Cumene Process, Ind Eng Chem Res 49:719-34 (2010).

The renewable benzene of the invention can be used as feedstocks to produce renewable phenol according to the cumene process (steps I-V) described above. In this case, the phenol produced has a renewable carbon content similar to that of the renewable benzene feedstock, since all the carbons in the phenol product comes from the renewable benzene feedstock. Thus, use of a renewable benzene feedstock that has about 100% renewable carbon content can yield a renewable phenol product that has a renewable carbon content of about 100%, and phenol with 100% renewable carbon content can be produced using a benzene feedstock having 100% renewable carbon. Alternatively, renewable cumene produced by a method of the invention can be used to produce renewable phenol and renewable acetone according to steps II-V of the cumene process described above. In this case, the renewable carbon content of the phenol product reflects the renewable carbon content of the phenyl group of the renewable cumene feedstock since all the carbons in the phenol product comes from the phenyl group of cumene. Thus, phenol with 100% renewable carbon content, as well as renewable acetone, can be produced using a cumene feedstock having 100% renewable carbons. Renewable phenol can be distinguished from non-renewable phenol in that the proportion of radiocarbon to total carbon in the renewable phenol is greater than that of non-renewable phenol alone. Catalysts that are useful for the conversion of benzene or cumene to phenol include phosphoric acid, a strong acid ion exchange resin or a zeolite in the hydrogen form.

The renewable phenol and acetone produced above can be condensed to produce Bisphenol-A (BPA) (structure below).

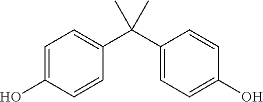 [SMILES: CC(C)(c1ccc(O)cc1)c1ccc(O)cc1]

In brief, two molecules of phenol are condensed with acetone in the presence of an acid catalyst such as HCl or a sulfonated polystyrene resin to produce BPA. An excess of renewable phenol can be used. Methods for producing BPA are known to those of skill in the art. See, for example, ORGANIC CHEMISTRY PRINCIPLES AND INDUSTRIAL PRACTICE 56-59, Green & Wittcoff, Weinheim:Wiley-VCH, 2003. The renewable BPA can have a renewable carbon content between about 20% (3 of 15 carbons are renewable carbons) to 100% (all carbons are renewable carbons). In addition, BPA with 100% renewable carbon content can be produced using a benzene feedstock having 100% renewable carbon. Renewable BPA can be distinguished from non-renewable BPA in that the proportion of radiocarbon to total carbon in the renewable BPA is greater than that of non-renewable BPA alone.

D. Renewable Benzoic Acid and Toluene Di-isocyanate from Renewable Toluene

Renewable toluene produced according to a method of the invention can be used as a feedstock for producing renewable benzoic acid and renewable toluene di-isocyanate. Benzoic acid can be produced by partial oxidation of toluene with O₂ as shown below.

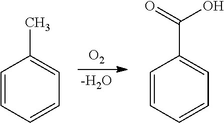

Catalysts that can be used for this reaction include cobalt or manganese naphthenates. Benzoic acid can be used as feedstock to produce a variety of chemicals including, for example, (1) benzoyl chloride, a precursor for benzyl benzoate used in artificial flavours and insect repellents; (2) benzoate plasticizers including the glycol-diethylengylcol- and triethyleneglycol esters; and (3) phenol, which can be used to produce cyclohexanol (by oxidative decarboxylation at about 300° C. to about 400° C. or about 200° C. with catalytic amounts of copper (II) salts) for use in nylon synthesis. Benzoic acid (and its salts including sodium, potassium or calcium salts) also can be used as a food preservative as it inhibits the growth of mold, yeast and bacteria. Benzoic acid also can be used therapeutically as topical antiseptics and inhalant decongestants. It can be used as a treatment for fungal skin diseases including tinea, ringworm and athlete's foot. Thus, renewable benzoic acid, as well renewable benzoyl chloride, renewable benzyl benzoate, renewable benzoate plasticizers, renewable phenol, and renewable cyclohexanol having 100% renewable carbon contents can be produced from a benzene feedstock having 100% renewable carbon. These renewable forms can be distinguished from their non-renewable counterpart based on their radiocarbon content. More specifically, the proportions of radiocarbon to total carbon for each of these renewable compounds are greater than those of their non-renewable counterparts.

Toluene di-isocyanate (TDI) exist in two isomeric forms: 2,4-TDI (CAS: 584-84-9) and 2,6-TDI (CAS: 91-08-7) as shown below.

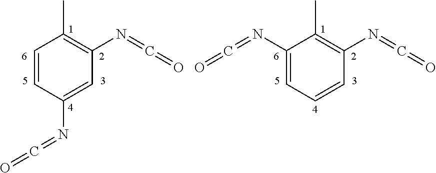

Methods of producing TDI are known to those of skill in the art. An example of a method for producing TDI is as follows. Generally, toluene is reacted with nitric acid in the presence of a catalyst to form dinitrotoluene (nitration reaction). Next, dinitrotoluene is reacted with hydrogen in the presence of a hydrogenation catalyst to form a mixture of isomers of toluene diamine (TDA). From the TDA mixture, meta-TDA is purified by distillation, dissolved in an inert solvent and reacted with phosgene (carbonyl chloride) to form a crude TDI mixture (phosgenation reaction). A 80:20 mixture of 2,4-TDI and 2,6-TDI, respectively, i.e. TDI (80/20), can be obtained by distillation of the crude TDI mixture. From TDI (80/20), pure 2,4-TDI and a 65:35 mixture of 2,4-TDI and 2,6-TDI, i.e. TDI (65:35) can be obtained by separation. See THE POLYURETHANES BOOK, eds. David Randall & Steve Lee, J. Wiley, New York (2002). TDI can be produced in the gas phase, as well as the liquid liquid phase. Methods for producing TDI in gas phase are described in, for example, U.S. Pat. Nos. 6,974,880 (Process for the Manufacture of (Poly-)isocyanates in the Gas Phase); 7,541,487 (Process for the Preparation of Isocyanates in the Gas Phase); and 7,615,662. Generally, the phosgenation is performed in the gas phase. The toluene diamine and phosgene are heated to more than 300° C. and then transferred in gaseous form to the reaction via a specially designed nozzle. Liquid TDI is condensed and purified by distillation and solvent and excess phosgene are recovered.

The renewable carbon content of TDI produced using toluene of the invention as feedstock is determined by the renewable carbon content of the renewable toluene feedstock. If all the carbons in the toluene feedstock are nenewable carbons, then all the carbons in the phenyl ring and the carbon in the methyl substituent of the TDI product are renewable carbons (the carbons in the isocyanates originate from phosgene). In this case, TDI has a renewable carbon content of 78% (7 of 9 carbons are renewable carbons). In contrast, if only the carbon in the methyl substituent of the toluene feedstock is a renewable carbon, then only the carbon in the methyl substituent of the TDI product is a renewable carbon, in which case TDI has a renewable carbon content of 11% (1 of 9 carbons are renewable carbons). If the renewable toluene feedstock for producing TDI is a mix of the two renewable forms of toluene, then the TDI product would have a renewable carbon content that reflects the renewable carbon content of the toluene mix. In this case, the renewable carbon content of the TDI product can be from at least about 11% to close to about 78%. Since renewable toluene in which all the carbons are renewable carbons is the predominant product of the transalkylation reaction, it can be used as a feedstock to produce TDI with a renewable carbon content close to 78%. In addition, TDI with 100% renewable carbon content can be produced from a benzene feedstock having 100% renewable carbon. Renewable TDI can be distinguished from non-renewable TDI in that the proportion of radiocarbon to total carbon in the renewable TDI is greater than that of non-renewable TDI alone.

Renewable TDI can be used for producing flexible polyurethane foam, which can be used in manufacturing upholstered furniture, mattresses and automotive seatings. Toluene diisocyanates can be used to synthesize polyurethane foams for use in furniture; bedding; insulation; in household refrigerators; for residential sheathing or commercial roofing; as insulation for truck trailers, railroad freight cars, and cargo containers; in polyurethane-modified alkyds as floor finishes, wood finishes, and paints; in moisture-curing coatings as wood and concrete sealants and floor finishes; in aircraft, truck, and passenger-car coatings.

E. Renewable Isophthalic Acid, Phthalic Anhydride & Terephthalic Acid from Renewable Xylenes of the Invention

Renewable xylenes of the invention, i.e. o-xylene, m-xylene and ρ-xylene, can be separated and converted to renewable phthalic anhydride, isophthalic acid and terephthalic acid, respectively, using methods known to those of skill in the art. The structures of phthalic anhydride, isophthalic acid and terephthalic acid are shown below.

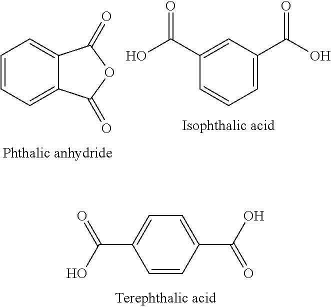

Renewable phthalic anhydride can be produced from renewable o-xylene of the invention in a catalytic oxidation reaction as follows: C₆H₄(CH₃)₂+3O₂→C₆H₄(CO)₂O+3 H₂O.

Similarly, renewable isophthalic acid and terephthalic acid can be produced from renewable m-xylene and ρ-xylene of the invention, respectively, using oxygen and a cobalt-manganese catalyst. The oxidation of ρ-xylene to terephthalic acid is as follows:

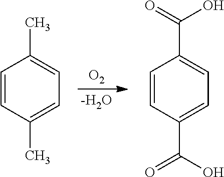

Oxidation can be performed with acetic acid as solvent and a catalyst composed of cobalt and manganese salts with bromide as promoter. Any impurity such as 4-formylbenzoic acid can be removed by hydrogenation, and highly pure terephthalic acid can be obtained by crystallization. Methods for producing phthalic anhydride, isophthalic acid and terephthalic acid are known in the art. See, for example, Richard J. Sheehan “Terephthalic Acid, Dimethyl Terephthalate, and Isophthalic Acid” in ULLMANN'S ENCYCLOPEDIA OF INDUSTRIAL CHEMISTRY, Wiley-VCH, Weinheim, 2005.

The renewable carbon contents of the phthalic anhydride, isophthalic acid and terephthalic acid products resemble that of the renewable xylene feedstocks because all the carbons in the products come from the xylene feedstocks. Thus, if only the carbons in the methyl substituents of the xylene feedstocks are renewable carbons, then the corresponding phthalic anhydride, isophthalic acid and terephthalic acid products will have a renewable carbon content of 25% (2 of 8 carbons, i.e. only the carbons on the functional groups, are renewable carbons). In contrast, if all the carbons in the xylene feedstocks are renewable carbons, then the phthalic anhydride, isophthalic acid and terephthalic products have a renewable carbon content of 100%, as all the carbons in the products are renewable carbons. If the xylene feedstocks are mixtures of the two renewable forms, then the corresponding phthalic anhydride, isophthalic acid and terephthalic acid products have renewable carbon contents that reflect the renewable carbon content of the mixtures, i.e. between about 25% and about 100%. Phthalic anhydride, isophthalic acid and terephthalic with 100% renewable carbon contents can be produced using a benzene feedstock having 100% renewable carbon. Renewable phthalic anhydride, isophthalic acid and terephthalic acid can be distinguished from their non-renewable forms in that the proportions of radiocarbons to total carbons in the renewable phthalic anhydride, isophthalic acid and terephthalic acid are greater than that of the corresponding non-renewable forms.

Renewable phthalic anhydride can be used to produce dyes such as quinizarin; organic reagents such as phthalimide and peroxy acid; and plasticizers for plastics. Renewable isophthalic acid can be used to produce polyethylene terephthalate bottle and fiber grade, unsaturated polyester resins and gelcoats, liquid and powder coating polyesters, alkyd resins, polyamides, and adhesives. Renewable terephthalic acid can be used to produce polyethylene terephthalate (PET), a theremoplastic polymer resin used in synthetic fibers; beverage, food and other liquid containers; thermoforming applications; and engineering resins.

F. Renewable Cyclohexane and Cyclohexanone from Renewable Benzene

Renewable benzene of the invention can be used as an industrial solvent, as well as in the production of drugs, plastics, synthetic rubber, and dyes. Renewable benzene of the invention also can be converted to renewable cyclohexane by reaction with hydrogen. In addition, renewable cyclohexane can be oxidized in air to form renewable cyclohexanone using a cobalt catalyst, for example. Methods for producing cyclohexane and cyclohexanone from benzene are known to those of skill in the art. See, for example, Michael T. Musser, Cyclohexanol and Cyclohexanone in ULLMANN'S ENCYCLOPEDIA OF INDUSTRIAL CHEMISTRY, Wiley-VCH, Weinheim, 2005. Generally, the catalytic hydrogenation of benzene to form cyclohexane can be performed using liquid or vapour-phase methods in the presence of a highly dispersed catalyst or in a catalytic fixed bed. Minimum reactor temperatures are preferred for maximum benzene conversion and minimum cyclohexane cracking.

The cyclohexane/cyclohexanone produced using renewable benzene of the invention as feedstock have renewable carbon contents resembling that of the renewable benzene feedstock because all the carbons in the cyclohexane/cyclohexanone come from the renewable benzene feedstock. Since the benzene of the invention can have a renewable carbon content anywhere from about 1% to about 100%, the cyclohexane/cyclohexanone produced from renewable benzene of the invention can also have a renewable carbon content anywhere from about 1% to about 100%. Cyclohexane/cylcohexanone having 100% renewable carbon content can be produced using a benzene feedstock having 100% renewable carbon. Renewable cyclohexane/cyclohexanone can be distinguished from their non-renewable forms in that the proportions of radiocarbons to total carbons in the renewable cyclohexane/cyclohexanone are greater than that of the non-renewable forms.

Renewable cyclohexane can be used as a solvent, oil extractant, paint and varnish remover, dry cleaning material, in solid fuels, and as an insecticide. Cyclohexane is used as a chemical intermediate and cyclohexane derivatives can be used for the synthesis of pharmaceuticals, dyes, herbicides, plant growth regulator, plasticizers, rubber chemicals, cycloamines and other organic compounds. Renewable yclohexanone can be used for producing adipic acid and caprolactam, both of which can be used to manufacture nylon, which is further processed into fibers for applications in carpeting, automobile tire cord, and clothing.

G. Renewable Aniline from Renewable Benzene

Renewable benzene of the invention can be used as feedstock to produce renewable aniline in two steps. First, benzene is nitrated using a concentrated mixture of nitric acid and sulfuric acid at 50° C. to 60° C. The nitrobenzene product is hydrogenated in presence of various metal catalysts. The reaction can be at 200° C. to 300° C. Alternatively, renewable benzene can be used as feedstock to produce phenol, which can be converted to aniline by reaction with ammonia. Methods for producing aniline from benzene are known to those of skill in the art. See, for example, Maxwell, Aniline and Nitrobenzene in SYNTHETIC NITROGEN PRODUCTS: A PRACTICAL GUIDE TO THE PRODUCTS AND PROCESSES, p. 361-71, Springer US (2006); Polinski & Harvey, Aniline Production by Dual Function Catalysis, Ind Eng Chem Prod Res Dev 10:365-69 (1971). Since the renewable carbon content of aniline is determined by that of the renewable benzene feedstock, renewable aniline can have a renewable carbon content anywhere from about 1% to about 100%. Thus, aniline having 100% renewable carbon content can be produced using a benzene feedstock having 100% renewable carbon. Renewable aniline can be distinguished from non-renewable aniline in that the proportion of radiocarbon to total carbon in the renewable aniline is greater than that of the non-renewable aniline.

Aniline can be used as feedstock to produce a variety of industrial chemicals. For example, aniline can be used to prepare methylene diphenyl diisocyanate (MDI), which is used to produce polyurethane. Aniline can also be used in rubber processing chemicals; herbicides; dyes (e.g. as precursor to indigo); and pigments. It is used to produce the antioxidant phenylenediamine and diphenylamine, drugs such as paracetamol, and dyes such as indigo.

H. Other Alkylated Benzenes from Renewable Benzene

The transalkylation of benzene with cymene can also generate renewable forms of other polyalkylated aromatic compounds including, for example, xylenes (o-, m- and p-xylene), cymene, dimethyl cumene, diisopropyl xylene, or tri-methyl-benzenes (1,2,3-trimethylbenzene, 1,2,4-trimethylbenzene, 1,3,5-trimethylbenzene), tetra-methyl-benzenes (1,2,3,4-tetramethylbenzene, 1,2,4,5-tetramethylbenzene and 1,3,4,5-tetramethylbenzene), penta-methyl-benzene, hexa-methyl-benzene, di-isopropyl-benzenes, tri-isopropyl-benzenes, and tetra-isopropyl-benzene.

I. Renewable Cresol and Terephthalic Acid from Renewable Cymene

Renewable ρ-cymene produce using a method of the invention can be used as a feedstock to produce renewable terephthalic acid or cresol using methods known in the art. Methods for producing terephthalic acid directly from oxidation of cymene involve the use of acetic acid and cobalt, manganese or bromide catalyst, as known in the art. In addition, cymene can be oxidized to cymene hydroperoxide, which is then converted to cresol upon peroxide cleavage. Thus, terephthalic acid or cresol having 100% renewable carbons can be produced using 100% renewable cymene as feedstock. Renewable terephthalic acid or cresol can be distinguished from non-renewable cresol in that the proportion of radiocarbon to total carbon in renewable cresol is greater than that in non-renewable cresol.

V. Recycling Benzene Produces Highly Renewable Benzene and Other Aromatic Compounds

The invention also provides a process for producing renewable benzene, which can be recycled as feedstock in a process of the invention or recovered as a product for sale in commerce. The following illustrates the process for producing highly renewable benzene.

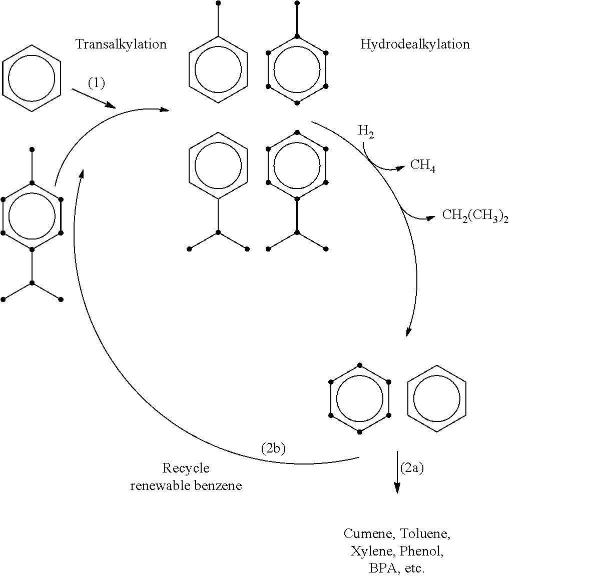

The first transalkylation using non-renewable benzene (1) and renewable ρ-cymene yields products with phenyl group from cymene and from non-renewable benzene. For example, the toluene and cumene products of the first transalkylation reaction are mixed populations of molecules some of which have renewable and others non-renewable phenyl groups. Dealkylation of such a mixture of different renewable forms (or a mixture of renewable and non-renewable forms), generates a benzene mixture of renewable and non-renewable forms. This benzene mixture can be used to make other renewable aromatic compounds (2a) or recycled as feedstock in subsequent transalkylation reactions with cymene (2b) resulting in transalkylation products with greater renewable carbon contents (broken arrows) than the products obtained from the first transalkylation reaction using non-renewable benzene. Continual recycling of benzene for transalkylation with renewable cymene generates benzene and alkylated products with 100% renewable carbons. The following table illustrates the rapidly increasing renewable carbon contents of benzene, toluene and cumene achieved by continual recycling of benzene.

| | Feedstock | | Products | |
|---|---|---|---|---|
| Reaction | ρ-cymene | benzene | Toluene/Benzene | Cumene |
| 1 | 2 units (R = 100)* | 2 units (R = 0)* | 2 units (R = 50)* | 2 units (R = 50) |
| 2 | 2 units (R = 100) | 2 units (R = 50) | 2 units (R = 75) | 2 units (R = 75) |
| 3 | 2 units (R = 100) | 2 units (R = 75) | 2 units (R = 87.5) | 2 units (R = 87.5) |
| 4 | 2 units (R = 100) | 2 units (R = 87.5) | 2 units (R = 93.75) | 2 units (R = 93.75) |
| 5 | 2 units (R = 100) | 2 units (R = 93.75) | 2 units (R = 96.875) | 2 units (R = 96.875) |

*“R” is the percentage of molecules that have phenyl groups derived from ρ-cymene (i.e. composed of renewable carbons). R = 50 indicates that half of the molecules contain phenyl groups derived from ρ-cymene.

A. Benzene

In the first transalkylation reaction, non-renewable benzene (1) reacts with renewable cymene, e.g., ρ-cymene, to produce toluene and cumene (and potentially other polyalkylated products). Assuming, for example, that about half of the alkylated products, e.g. toluene or cumene, will have renewable phenyl groups from cymene, while the other half will have non-renewable phenyl groups from benzene, recovery and dealkylation of an alkylated product such as toluene, for example, yields benzene, half of which is renewable benzene (from cymene) and half is non-renewable benzene. Thus, benzene has a renewable carbon content of about 50% after the first transalkylation and hydrodealkylation reactions.

Use of this benzene (now about 50% renewable carbons) for transalkylation with 100% renewable cymene yields products in which about 75% of the molecules are composed of renewable phenyl groups, i.e. (0.5×100%)+(0.5×50%)—about half of the molecules have phenyl groups from cymene (100% renewable carbons) and about half of the molecules have phenyl groups from the benzene (50% renewable carbons). Dealkylation of these products yields benzene in which about 75% of the molecules are composed of renewable carbons. Use of this benzene (now about 75% renewable carbons) for transalkylation with renewable cymene yields products in which 87.5% of the molecules are composed of renewable phenyl groups, i.e. (0.5×100%)+(0.5×75%). Dealkylation of these products yields benzene in which 87.5% of the molecules are composed of renewable carbons, i.e. a renewable carbon content of 87.5%. Use of this benzene for transalkylation with renewable cymene yields products in which 93.8% of molecules are composed of renewable phenyl groups, i.e. (0.5×100%)+(0.5×87.5%). Dealkylation of these products yields benzene having a renewable carbon content of 93.8%. Use of this benzene for transalkylation with renewable cymene yields products in which 96.9% of molecules are composed of renewable phenyl groups, i.e. (0.5×100%)+(0.5×93.8%). Dealkylation of these products yields benzene having a renewable carbon content of 96.9%. Further recycling/reuse of benzene for transalkylation with renewable cymene results in benzene with 100% renewable carbon content, which can be used as a feedstock in a method of the invention to produce aromatic compounds that have 100% renewable carbons.

B. Cumene

The above table also shows that the repeated recycling/reuse of renewable benzene coupled with continual feeding of renewable ρ-cymene into the transalkylation process produces cumene with increasing renewable carbon content. Thus, in another embodiment, the invention provides a process for producing highly renewable cumene, toluene (and aromatic compounds derived from these) from renewable benzene and renewable cymene as illustrated below for ρ-cymene.

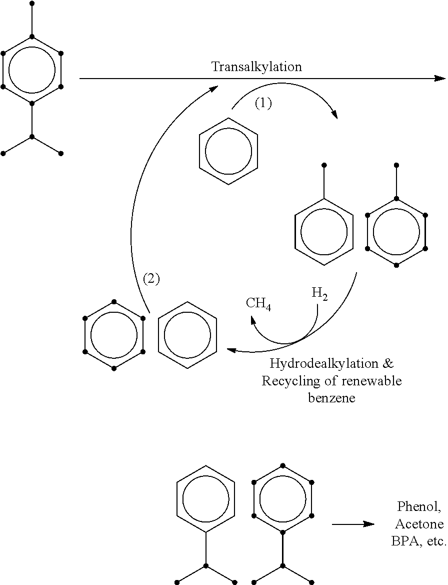

The solid arrows illustrate the first transalkylation reaction of non-renewable benzene (1) with renewable ρ-cymene, while the broken arrows illustrate the recycling the renewable benzene back in the transalkylation with renewable cymene (2). In a transalkylation reaction in which the methyl substituent of cymene is as likely as the isopropyl substituent of cymene to be transferred to non-renewable benzene, the toluene and cumene products are as likely to be composed of renewable phenyl groups from cymene as non-renewable phenyl groups from benzene. In this case, the first transalkylation reaction yields toluene in which about half of the molecules have renewable phenyl groups from cymene. Similarly, about half of the cumene molecules produced from the first transalkylation reaction have renewable phenyl groups from cymene (the other half have non-renewable phenyl groups from benzene). Thus, in the above table, the toluene and cumene products of the first transalkyation have R values of 50% indicating that half of the molecules in each product have renewable phenyl groups (from cymene). In the second transalkylation reaction, renewable benzene (R=50 because it comes from dealkylation of the R=50 toluene product of the first transalkylation reaction) is reacted with renewable cymene (R=100). The resulting toluene and cumene products have R values of 75%, i.e. 75% of the molecules have renewable phenyl groups—half of the molecules produced will have phenyl groups that come from renewable cymene (R=100) and half will have phenyl groups that come from renewable benzene (R=50), i.e. (0.5×100%)+(0.5×50%). In the third transalkylation reaction, renewable benzene (R=75 because it comes from dealkylation of the R=75 toluene product of the second transalkylation reaction) is reacted with renewable cymene (R=100). The resulting toluene and cumene products have R values of 87.5%, i.e. (0.5×75%)+(0.5×100%). In the fourth transalkylation reaction, renewable benzene (R=87.5 because it comes from dealkylation of the R=87.5% toluene product of the third transalkylation reaction) is reacted with renewable cymene (R=100). The resulting toluene and cumene products have R values of 93.8%, i.e. (0.5×100%)+(0.5×87.5%). In the fifth transalkylation reaction, renewable benzene (R=93.8 because it comes from dealkylation of the R=93.8% toluene product of the third transalkylation reaction) is reacted with renewable cymene (R=100). The resulting toluene and cumene products have R values of 96.9%, i.e. (0.5×100%)+(0.5×93.8%). Since the R value reflects renewable carbon contents, the values in the above table illustrate that the renewable carbon contents of the transalkylation products increase rapidly as benzene is “recycled” by (1) continual recovery of renewable toluene, (2) conversion to renewable benzene, and 3) reuse of the renewable benzene in subsequent transalkylations. Further recycling of benzene for transalkylation with 100% renewable cymene results in cumene and toluene products that have 100% renewable carbon contents.

If the transalkylation reaction yields primarily toluene with 100% renewable carbons (7 of 7 carbons are renewable), conversion of this toluene to benzene by hydrodealkylation or disproportionation yields primarily renewable benzene in which all the carbons are renewable carbons. Use of this benzene for transalkylation with ρ-cymene produces cumene with a renewable carbon content greater than that of the cumene produced in the first transalkylation reaction when non-renewable benzene is used. As the renewable carbon content of the benzene feedstock increases with subsequent transalkylation, the renewable carbon content of the cumene also increases. If the recycled benzene feedstock has more than 50% renewable carbons, the cumene product will also have more than 50% renewable carbons. More specifically, transalkylating a benzene feedstock having equal numbers of renewable and non-renewable benzenes with 100% renewable cymene produces cumene with 66.6% renewable carbons, i.e. (0.5×100%)+(0.5×33%) since half the cumene will be composed of 100% renewable and half of the cumene molecules will be composed of 33% renewable carbons. In contrast, transalkylating a benzene feedstock composed of 6 renewable benzenes for every 4 non-renewable benzenes yields a cymene product with 73.2% renewable carbons, i.e. (0.6×100%)+(0.4×33%). Thus, transalkylating a benzene feedstock having more renewable benzenes than non-renewable benzenes with 100% renewable cymene produces cymene with more than 66.6% renewable carbons. In this case, the cymene product consists of two renewable forms. As the proportion of renewable benzene in the feedstock increases, the renewable carbon content of the cumene product also increases so that when the benzene feedstock consists of 100% renewable benzene, the cumene product is also 100% renewable cumene. Thus, benzene having 100% renewable carbon content can be generated by a process that involves (1) the initial transalkylation of non-renewable benzene with 100% renewable cymene, (2) dealkylating the transalkylation products to generate benzene composed of renewable and non-renewable forms, (3) repeated recycling of this benzene in subsequent transalkylation reactions with 100% renewable cymene. Further, continual recycling of benzene in combination with transalkylation using 100% renewable ρ-cymene can produce cumene and toluene products that have 100% renewable carbon contents.

If the transalkylation reaction primarily yields a renewable cumene with 100% renewable carbons (9 of 9 carbons are renewable carbons from cymene), the renewable cumene can be converted to renewable benzene or used directly to produce other renewable aromatics compounds as described here.

VI. Transalkylation of Benzene with Cymene

The feedstock for the transalkylation process of the invention includes non-renewable or renewable aromatic compounds such as non-renewable or renewable benzene and a renewable transalkylating agent that is a polyalkyl aromatic hydrocarbon containing two or more alkyl groups, each of which can be, independently, a renewable methyl group or a renewable isopropyl group. An example of a useful transalkylating agent is renewable cymene, for example, ρ-cymene (4-isopropyltoluene). Thus, the combined feed stream for the transalkylation process generally comprises alkylaromatic hydrocarbons of the general formula C₆H₍₆₋ₙ₎Rn, where n is an integer from 0 to 5 and each R can be, independently, CH₃, C₃H₇, or any combination of CH₃ or C₃H₇. For example, the combined feed stream to the transalkylation process can include non-renewable or renewable benzene and renewable cymene. It can also include the polyalkylated aromatic products of the transalkylation of benzene with cymene such as, without limitation, dimethyl cumene, diisopropyl xylene, or tri-methyl-benzenes (1,2,3-trimethylbenzene, 1,2,4-trimethylbenzene, 1,3,5-trimethylbenzene), tetra-methyl-benzenes (1,2,3,4-tetramethylbenzene, 1,2,4,5-tetramethylbenzene and 1,3,4,5-tetramethylbenzene), penta-methyl-benzene, hexa-methyl-benzene, di-isopropyl-benzenes, tri-isopropyl-benzenes, tetra-isopropyl-benzene and di-isopropyl toluene.

The non-renewable benzene feedstock for the transalkylation reaction can be produced synthetically, for example, from naphtha by catalytic reforming or by pyrolysis followed by hydrotreating to yield an aromatics-rich product. The feedstock may be derived from such product with suitable purity by extraction of aromatic hydrocarbons from a mixture of aromatic and non-aromatic hydrocarbons and fractionation of the extract. For instance, aromatics may be recovered from a reformate stream. The reformate stream may be produced by any of the processes known in the art. The aromatics then may be recovered from the reformate stream with the use of a selective solvent, such as one of the sulfolane type, in a liquid—liquid extraction zone. The recovered aromatics may then be separated into streams having the desired carbon number range by fractionation. When the severity of reforming or pyrolysis is sufficiently high, extraction may be unnecessary and fractionation may be sufficient to prepare the feedstock. Renewable benzene can be recovered as a product of a transalkylation process of the invention. It can also be recovered as a product of a hydrodealkylation process of the invention, or as a product of a toluene disproportionation process as provided by the invention. Renewable benzene can be composed of 100% renewable benzene (all the carbons are renewable carbons) to benzene that consist of varying amounts of renewable and non-renewable benzenes. As such the renewable carbon contents of renewable benzene can vary from more than 0% to 100%.

The transalkylation agent can be renewable cymene synthesized biologically or produced from the desaturation (aromatization) of a biologically-synthesized, thus renewable, cyclic monoterpene. The monoterpene can be isolated from natural sources (e.g. bacteria, fungi, algae, plants, insects and higher animals) or can be produced from a recombinant organism as discussed above. The monoterpene can be converted to cymene in a biological process using an enzyme such as a dehydrogenase or oxidase or in a chemical dehydrogenation process using a catalyst, for example, a metal catalyst such as cobalt, cadmium, nickel, platinum, palladium, another noble metal, or a mixture of these metals. Thus, renewable cymene can be obtained by the dehydrogenation of a monoterpene using, for example, one or more of the following metals: ruthenium, rhodium, palladium, silver, osmium, iridium, platinum and gold. To facilitate conversion of the monoterpene to cymene by dehydrogenation, hydrogen can be removed as it is formed during the dehydrogenation of the monoterpene to cymene. The transalkylation agent can also be a renewable product, i.e. a polyalkylated aromatic, obtained by separation of the products of a transalkylation process of the invention. The renewable polyalkylated aromatic can be recycled as a transalkylation agent for a subsequent transalkylation reaction. For example, the transalkylation agent can be cymene formed after transalkylation of both the methyl and isopropyl substituents of biologically-synthesized ρ-cymene to the non-renewable benzene feedstock. Thus, in some embodiments, in addition to ρ-cymene, the transalkylation agent can also be, without limitation, m-cymene, o-cymene or di-isopropyl benzene.

The molar ratio of renewable or non-renewable benzene to renewable polyalkyl aromatic hydrocarbon in the feed stream can range from about 1:1 to about 50:1, for example, about 2:1 to about 20:1. The molar ratio of benzene to polyalkyl aromatic hydrocarbon in the feed stream can be, for example, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, or about 50:1. Thus, the polyalkylated aromatic transalkylation agent can be present in an amount that is 50 weight % or less of the feed. The benzene can be non-renewable benzene or renewable benzene having a renewable carbon content from more than 0% to 100%. Renewable benzene can be distinguished from non-renewable benzene as the proportion of radiocarbon to total carbon in renewable benzene is greater than that of non-renewable benzene.

The transalkylation reaction can be performed in gas phase, in liquid phase, in partial liquid phase (as a mixture of gas and liquid). Transalkylation can also be performed using any combination of gas phase, liquid phase or partial liquid phase transalkylation. For example, transalkylation can be performed in more than one zone, for example, in two zones, one of which can be in the gas phase and the other in liquid phase or partial liquid phase. When the transalkylation reaction is performed in liquid phase, the reaction temperature can be from about 100° C. to about 540° C., and the reaction pressure can be moderately elevated ranging from about 100 kPa to about 6 MPa absolute. Transalkylation can be performed at a wide range of space velocities, for example, the weight hourly space velocity can be from about 0.1 to about 20 hr⁻¹. A useful set of liquid phase transalkylation conditions can be, for example, a temperature from about 200° C. to about 300° C., a pressure from about 10 kg/cm² to about 50 kg/cm², and a weight hourly space velocity from about 0.5 to about 15 hour⁻¹. Another useful set of transalkylation condition is a temperature from about 250° C. to about 500° C., a pressure from about 10 to about 65 kg/cm², and a weight hourly space velocity from about 1.0 to about 10 hr⁻¹. When the transalkylation reaction is performed in partial liquid phase, the reaction temperature can range from about 38° C. (100° F.) to about 315° C. (600° F.), for example, from about 121° C. (250° F.) to about 232° C. (450° F.). The reaction pressure should be sufficient to maintain at least a partial liquid phase, typically in the range of about 50 psig to about 1000 psig, for example, about 300 psig to about 600 psig. The weight hour space velocity can range from about 0.1 to about 10. When the transalkylation reaction is performed in gas phase, the combination of pressures and temperatures are selected such that the feed and products are all in the gas phase. Non-limiting examples of gas phase operating conditions include: a pressure of 100-500 psig, e.g. 200 to 400 psig; a weight hour space velocity (WHSV) of between 0.5-10 hr⁻¹, e.g., between 1.5 and 4.0 hr⁻¹; a reaction temperature of 500-900° F., e.g. between 550° F. and 800° F., and a H₂/HC feed mole ratio of between 1 and 10, e.g. between 2 and 5. The phase for these hydrocarbons depends on the combination of temperature and pressure, which can be determined by a chemical engineer of ordinary skill in the art using standard methods. When transalkylation is performed in liquid phase, it can be carried out in a substantial absence of hydrogen, i.e. without the addition of hydrogen beyond what may already be present and dissolved in a typical liquid aromatic feedstock. When the transalkylation reaction is performed in partial liquid phase, hydrogen can be added in an amount less than 1 mole per mole of aromatics. When transalkylation is performed in gas phase, hydrogen can be added with the feedstock in an amount from about 0.1 moles per mole of aromatics up to 10 moles per mole of aromatics.

The feed stream into the transalkylation reactor can be heated by indirect heat exchange against the effluent of the transalkylation reaction and then heated to reaction temperature by exchange with a warmer stream, steam or a furnace as well known to those of skill in the art. The feed stream can be passed through a transalkylation reaction zone containing one or more individual transalkylation reactors. The details of heat exchange and flow details are well known to the art.

Various types of reactors can be used for the transalkylation process of the invention. For example, the transalkylation can be performed in a batchwise fashion by adding the catalyst and aromatic feedstock to a stirred autoclave, heating to a selected reaction temperature and then adding the transalkylation agent. A heat transfer fluid can be circulated through the jacket of the autoclave or a condenser can be provided to remove the heat of reaction and maintain a constant temperature. In addition, a single reaction vessel with a fixed cylindrical bed of catalyst, as well as reaction configurations that involves moving beds of catalyst or radial-flow reactors can be used. The fixed bed reactor can operate in an upflow or downflow mode, while the moving bed reactor can operate with concurrent or countercurrent catalyst and hydrocarbon flows. The moving bed reactor can be used for continuous removal of spent catalyst and regeneration and replacement by fresh or regenerated catalyst. Thus, transalkylation reactors can contain a single catalyst bed or multiple beds and can be equipped for the interstage addition of the transalkylating agent and interstage cooling. Interstage cooling can be accomplished using a cooling coil, heat exchanger or by staged addition of aromatic feedstock, that is addition of the feedstock in at least two stages. In this embodiment, at least a portion of the aromatic feedstock is added between the catalyst beds or reactors. When hydrogen is added to a transalkylation unit, i.e. in the case of gas-phase transalkylation, the unit can include a recycled gas compressor for recycling hydrogen recovered from the reactor effluent in a separator vessel. Thus, where transalkylation is performed in gas-phase, a hydrogen gas phase recycle loop systems is required around the reactor system.

Catalysts that can be used for transalkylation are known to those of skill in the art. U.S. Pat. No. 3,849,340, for example, describes a catalytic composite useful for transalkylating alkylaromatic hydrocarbons that is composed of a mordenite component having a SiO₂/Al₂O₃ mole ratio of at least 40:1 and a metal component that can be copper, silver or zirconium. U.S. Pat. No. 5,763,720 describes transalkylation using a catalyst composed of a zeolite, e.g. MCM-22, ZSM-12 and Zeolite beta, and a hydrogenation component. Non-limiting examples of zeolites that can be used for transalkylation include zeolite beta (see, e.g., U.S. Pat. Nos. 3,308,069 and 4,891,458); zeolite MTW; both cubic and hexagonal forms of zeolite Y (see, e.g., U.S. Pat. No. 3,130,007); zeolite X; mordenite (see, e.g., Donald W. Breck in ZEOLITE MOLECULAR SIEVES, pages 122-124 & 162-163, John Wiley and Sons (1974)); zeolite L (see, e.g., U.S. Pat. No. 3,216,789); zeolite ferrierite; MFI; erionite; zeolite SSZ-26, zeolite Al-SSZ-33, zeolite CIT-1, zeolite SSZ-35, and zeolite SSZ-44. Information on zeolite that can be used in a method of the invention can be found in the Database of Zeolite Structure, available at http://www.iza-structure.org/databases/. Zeolite catalysts that can be used in a method of the invention are also discussed by Tsai et al., Disproportionation and Transalkylation of Alkylbenzenes, Applied Catalysis A: General 181: 355-98 (1999).

In some embodiments, the transalkylation reaction can be performed in the same reactor as the dehydrogenation step for conversion of a monoterpene to cymene. In these cases, a catalyst with multiple functions, e.g. dehydrogenation and transalkylation functions, or a mixture of catalysts can be used. For example, the multifunctional catalyst can have surface oxidation sites that are readily accessible by the monoterpene molecules for catalyzing dehydrogenation of monoterpene to cymene and acidic sites in geometrically confined pores that are readily accessible to aromatic molecules such as benzene, toluene, cymene and cumene for transalkylation. For example, the acidic sites can be in geometrically confined pores with openings about 5 Angstrom to about 7 Angstrom. Additional catalysts that can be used for transalkylation include a zeolite of the faujasite structure in the hydrogen form, a hydrogen mordenite, or another hydrogen zeolite with a pore diameter of about 5.2 to about 7.8 Angstrom. A dealuminated HY zeolite, for example, can be used.

To fabricate the catalyst, a refractory binder or matrix can be used to provide strength and reduce costs. The binder is generally uniform in composition and relatively refractory to the conditions used in the process. Suitable binders include, for example, inorganic oxides such as one or more of alumina, magnesia, zirconia, chromia, titania, boria, thoria, phosphate, zinc oxide and silica. Inclusion of a binder or matrix in the catalyst is optional. Where a binder or matrix is used in the fabrication of the catalyst, the binder can be in the range of about 5 weight-% to about 95 weight-% of the catalyst, while the zeolite can be in the range of about 5 weight-% to about 99 weight-% of the catalyst. Thus, a transalkylation catalyst can be, for example, a type Y zeolite having an alumina or silica binder or a beta zeolite having an alumina or silica binder. The catalyst can contain an optional metal component. The metal component can be a Group VIII (IUPAC8-10) metal, for example, a platinum-group metal such as platinum, palladium, rhodium, ruthenium, osmium and iridium. The metal component can also be rhenium. The optional metal component can exist within the catalytic composite as a compound such as an oxide, sulfide, halide, or oxyhalide. It can exist in chemical combination with one or more of the other ingredients of the composite or as an elemental metal. This component can be present in the final catalyst composite in any amount that is catalytically effective, generally about 0.01 to about 2 weight-% of the final catalyst calculated on an elemental basis. The component can be incorporated into the catalyst in any suitable manner such as coprecipitation or cogelation with the carrier material, ion exchange or impregnation. Impregnation can be performed using water-soluble compounds of the metal, for example, with chloroplatinic acid or perrhenic acid. Rhenium can also be used in conjunction with a platinum-group metal. The catalyst can also contain an optional modifier component. Examples of metal modifier components include, without limitation, tin, germanium, lead, indium, and mixtures thereof. Catalytically effective amounts of the metal modifiers can be in a range of about 0.01 weight-% to about 2.0 weight-% on an elemental basis. The metal modifiers can be incorporated into the catalyst by any suitable manner. In addition, a typical water concentration of less than about 200 wt-ppm is present.

Passage of the combined feed stream through the transalkylation zone results in production of an effluent stream composed of unconverted feed stocks and reaction products. Reaction products that can be obtained from a transalkylation process of the invention include toluene and cumene, as well as polyalkylated aromatics such as xylene and diisopropylbenzene. The effluent from the transalkylation reaction can have a cumene content from at least about 0.1 weight % to about 99% weight. The effluent from the transalkylation reaction can also have similar toluene content. The transalkylation reaction can proceed to equilibrium to achieve about 90 weight % or greater selectivity to monoalkylated products such as toluene and cumene. The weight % yield of a product of the transalkylation reaction can be calculated on a net effluent basis. The monoalkylated products cumene and toluene, polyalkylated products, as well as any excess aromatic feedstock and transalkylating agent in the transalkylation effluent can be separated by distillation. Benzene in the transalkylation effluent can be recovered by distillation, and the bottom fraction of the benzene distillation is further distilled to separate each of the monoalkylated products from the polyalkylated aromatics. The monoalkylated aromatics such as toluene and cumene can be recovered as product, while the excess benzene feedstock and polyalkylated aromatics can be recovered and reuse in subsequent transalkylation reaction. For example the excess benzene feedstock and polyalkyated aromatics can be recycled to the transalkylation reactor to undergo transalkylation, or the polyalkylated aromatics can be reacted with additional renewable or non-renewable benzene feedstock in a second or separate reactor. In the latter embodiment, the bottoms from the distillation of monoalkylated products can be combined with a stoichiometric excess of the renewable or non-renewable benzene feedstock and allowed to react in a separate transalkylation reactor over a suitable transalkylation catalyst as discussed above. The recovered toluene and cumene products can be very pure, for example, 99% or greater than 99% purity and less than 500 ppm of other aromatics.

More specifically, the effluent can be cooled by indirect heat exchange against the feed stream entering the reaction zone and then further cooled through air or cooling water. The effluent can be passed into a stabilizer or stripping or separation column. The transalkylation effluent can be separated into a benzene stream, which can be recycled for use for additional transalkylation, and a mixed C₇ and heavier aromatic stream containing toluene, xylene, cumene, cymene and other polyalkylated aromatics. The C₇ and heavier aromatic stream can be further separated into a toluene stream and a C₈ and heavier aromatic stream containing xylene, cumene, cymene and other polyalkylated aromatics. The toluene can be recovered as a product or used as feed stock in (1) a toluene disproportionation reaction to produce xylene and benzene or (2) a hydrodealkylation reaction as discussed herein to generate benzene and methane. The C₈ and heavier aromatic stream can be separated by a third column into a xylene stream and a C₉ and heavier aromatic stream containing cumene, cymene and other polyalkylated aromatics. The xylene can be recovered as a product of the transalkylation or used as a feedstock for a hydrodealkylation reaction to generate benzene and methane. The C₉ and heavier aromatic stream can be separated by a fourth column into a cumene stream and a C₁₀ and heavier aromatic stream containing cymene and polyalkylated products. The cumene can be recovered as a product of the transalkylation and used as feedstock to produce numerous industrial chemicals including phenol, bisphenol-A and α-methylstyrene. The C₁₀ and heavier aromatic stream containing cymene and polyalkylated products can be partially or totally recycled to the transalkylation reaction zone.

VII. Production of Renewable Xylenes by Toluene Disproportionation

Renewable toluene of the invention can be used as feedstock to produce xylene and benzene in a disproportionation reaction according to methods known to those of skill in the art. Disproporationation can be performed using transalkylation catalysts described above. The catalysts can also be one that is shape selective allowing for the production of ρ-xylene in excess of the equilibrium concentration. Disproporation can be performed using zeolites having medium pore size such as ZSM-5. Examples of useful catalysts include ZSM-5 in the hydrogen form, partially metal ion exchanged ZSM-5, the hydrogen form of other zeolites with the MEI structure or a partially metal ion exchanged form fo other zeolites with the MFI structure. Numerous methods for toluene disproportionation including selective toluene disproportionation processes and reaction conditions are described by Tsai et al., Disproportionation and Transalkylation of Alkylbenzenes, Applied Catalysis A: General 181: 355-98 (1999). Methods for performing toluene disproportion are described further by Han et al., Oil Gas J. 21: 83 (1989); Gorra et al., Oil Gas J. 12: 60 (1992); Chang & Shihabi, U.S. Pat. No. 5,243,117 (1993); Johnson et al., 22nd Annual DeWitt Petrochemical Review, Houston, Tex., Mar. 18-20, 1997; and Menard, Oil Gas J. 16: 46 (1987).

VIII. Production of Renewable Benzene and Methane by Dealkylation of Toluene or Xylene

Renewable toluene or xylene of the invention (as well as other polyalkylated aromatic products of the transalkylation reaction) can be dealkylated to produce renewable benzene and renewable methane (or renewable propane) using methods known to those of skill in the art. The dealkylation can be achieved in a thermal or catalytic process. Dealkylation can be performed in the presence of H₂ (hydrodealkylation) or steam (steam dealkylation). In hydrodealkylation, molecular hydrogen (H₂) can be from the oxidation of monoterpene to cymene or obtained from a separate source. Dealkylation catalysts that can be used include metal and oxide catalysts. Useful metals include, without limitation, noble metal catalysts, Group VIII metals such as Rh (rhodium), Ir (iridium), Os (osmium), Ru (ruthenium), Pt (platinum), and Pd (palladium), as well as Ni (nickel), Mo (molybdate), and bimetal catalysts such as Rh—Pt, other noble metals such as Ag (silver) and Au (gold) as well as other group VIII metals such as Fe (iron) and Co (cobalt). Catalysts can, optionally, include a metal support such as Al₂O₃, SiO₂ or C. Specific examples of hydrodealkylation catalysts include, without limitation, Ru, Rh, Pd, Os, Ir, Pt, Ni, Re or bimetal Rh—Pt on a γ-Al₂O₃ support, a SiO₂ support or a C support. Other non-limiting examples include chromium or molybdenum oxides, and platinum or platinum oxides, supported on silica or alumina, in particular, Cr₂O₃/Al₂O₃, Mo₂O₃/alumina or CoO/alumina. The metal component in the catalyst can be present in an amount from about 1% to about 15%. The conditions for dealkylation include a temperature range from about 200° C. to about 800° C., e.g. about 400° C. to about 600° C., and a pressure range from about 10 atmosphere to about 90 atmosphere, e.g. about 20 atmosphere to about 60 atmosphere depending on whether a catalytic or thermal process is used. An alternative condition for hydrodealkylation include a temperature of about 350° C. to about 700° C. and a pressure of about 5 to 100 atmospheres, or a temperature of about 450° C. to about 650° C. and a pressure of about 15 to 70 atmospheres. Generally, the dealkylation process involves first heating the feedstock, i.e. toluene, xylene and/or other poly-alkyl aromatic compounds, then passing the hot feedstock through a dealkylation reactor containing a catalyst for dealkylation. The resulting effluent is passed through a hydrogen sripper to remove hydrogen, which can be recycled to the dealkylation reactor, and then separated by fractionation to recover the benzene product. The unconverted toluene and other aromatic compounds can be recycled. Catalysts for hydrodealkylation can be obtained from Sud-Chemie AG, United Catalyst, and Engelhard. Methods of preparing catalysts and performing dealkylation are known to those of skill in the art. See, for example, Grenoble, The Chemistry and Catalysis of the Toluene Hydrodealkylation Reaction, Journal of Catalysis 56: 32-9 (1979); see also Al-Khowaiter, J. King Saud Univ., Vol. 8, Science (2), pp. 207-221 (A.H. 1416/1996); Golubyatnikov et al., Hydrodealkylation of Toluene Using Hydrogen-rich Reformer Gas, Chemistry and Technology of Fuels and Oils 23:418-20 (1987); Shull & Hixson, Kinetics of Thermal Hydrodealkylation of Mesitylene, m-Xylene and Toluene, Ind Eng Chem Process Des Dev 5:146-50 (1966).

IX. Integrated Aromatic Complex of the Invention

The invention provides an integrated aromatics complex incorporating the transalkylation unit of the present invention along with a reforming unit, a benzene separation unit, a toluene separation unit, an alkyl-aromatic isomerization unit, a xylene separation unit, a cumene separation unit, as well as an optional second transalkylation unit, an optional cyclic monoterpene dehydrogenation unit, an optional toluene disproportionation unit and an optional hydrodealkyation unit. The reforming unit can be used to generate benzene. The cyclic monoterpene dehydrogenation unit can be used to generate renewable cymene and hydrogen. Benzene is transalkylated in with renewable cymene (and other, recycled, C₁₀ and heavier aromatics) to form renewable toluene and renewable cumene (as well as other polyalkylated aromatics) in the transalkylation unit. The benzene separation unit can be used to separate the renewable and non-renewable benzene from heavier alkylated aromatic compounds. The toluene separation unit can be used to separate the renewable toluene from other heavier alkylated aromatic compounds. The xylene separation unit can be used to separate renewable xylene from other heavier alkylated aromatic compounds. The cumene separation unit can be used to separate renewable cumene from heavier alkylated aromatic compounds. Renewable toluene can be further transalkylated in the optional second transalkylation unit to form renewable xylenes and renewable benzene. The renewable benzene can be recycled back to the transalkylation unit, while the renewable xylenes can be processed in a loop, i.e. a ρ-xylene production unit that includes the combination of an isomerization unit and a ρ-xylene separation unit. The ρ-xylene separation unit can be either a crystallization or adsorptive based separation process well known to the art. The ρ-xylene separation unit selectively removes the ρ-xylene in high purity from a non-equilibrium mixture of other xylenes, which can be contacted with an alkylaromatic isomerization catalyst in another process well-known in the art. The isomerization process re-equilibrates the mixture back to an equilibrium amount of ρ-xylene, which can be recycled back to the ρ-xylene separation unit for further recovery. Renewable toluene also can be converted to renewable benzene and renewable methane in the optional hydrodealkylation unit or converted to renewable xylene and renewable benzene in the optional disproportionation unit. The renewable benzene and renewable xylene can be used as discussed above, while the renewable methane can be used as a fuel directly or, for example, steam reformed to produce carbon monoxide and hydrogen (H₂) for use in Fischer-Tropsch synthesis of alkanes. Renewable cumene can be recovered as used to produce a variety of chemicals including phenol and bis-phenol A using methods known to those of skill in the art. Similarly, the renewable benzene, toluene, xylenes also can be recovered as used as feedstock to produce a variety of chemicals and polymers using methods known to those of skill in the art.

X. Bio-Aromatic Products of the Invention and their Uses

In the present invention, cyclic monoterpenes such as, limonene, carene, cineole, phellandrene (α- and β-), pinenes (α- and β-) and terpinenes (α-, β- or γ-), which can be produced or isolated from renewable feedstocks such as recombinant organisms, plants or trees, can be converted to renewable cymene by dehydrogenation. The renewable cymene can be used to produce renewable cumene, renewable toluene and a variety of related renewable aromatic compounds, any of which can be (1) withdrawn as a product to be sold in commerce, (2) recycled in a process of the invention, or (3) used as feedstock to produce other renewable aromatic chemicals as provided by the invention and in accordance with its market value and demand.

According to the invention, renewable ρ-cymene can be transalkyated with benzene to produce renewable cumene (i.e. Bio-Cumene) and renewable toluene (i.e. Bio-Toluene). Bio-Cumene can be recovered as a product of the transalkylation reaction and used as a feedstock to produce renewable phenol (Bio-Phenol), acetone, as well as a variety of biogenic products derived from these including phenolic resins, germicidal paints, adhesives, coatings, polycarbonate, bisphenol A, pharmaceuticals and solvents.

Bio-Toluene can be recovered as a product of the transalkylation and can be used to in medicine, paint solvents, explosives, as a gasoline component, to produce renewable toluene diisocyanate (Bio-Toluene-diisocyanate or Bio-TDI), which in turn can be used to manufacture explosives (TNT), as well as biogenic polyurethane for use in a variety of products including, for example, foam bedding, cushions car seats, insulation and refrigerators. Bio-Toluene can also be used as a feedstock to produce renewable xylenes (Bio-Xylenes or Bio-m-Xylene, Bio-o-Xylene and Bio-ρ-Xylene) by toluene disproportionation as provided by the invention. Bio-ρ-Xylene can be recovered as a product and then used to produce renewable terephthalic acid (Bio-Terephthalic Acid or Bio-TPA) by catalytic oxidation. Bio-TPA can be used to manufacture biogenic polyester fibers and resins for use in a variety of products including for example, apparel, carpet, upholstery, cords, fire hoses and belts. Bio-o-Xylene can be used to produce renewable phthalic anhydride (Bio-Phthalic Anhydride or Bio-PA), which can be used to make a variety of biogenic products including plasticizers, pharmaceuticals and as chemical intermediates. Bio-m-Xylene can be used to produce renewable isophthalic acid (Bio-Isophthalic acid) which can be used to manufacture products such as plasticizers, azo dyes and wood preservers. Bio-Toluene can also be used to produce renewable benzene (Bio-Benzene) and methane (Bio-Methane) by hydrodealkylation as provided by a method of the invention. Bio-Benzene can be recycled in subsequent transalkylation to produce cumene and toluene with even greater renewable carbon contents. Bio-Benzene can be recovered as a product and then used to produce Bio-Phenol by methods known to those of skill in the art including, for example, the cumene process. Bio-Benzene can also be used to produce Bio-Cumene, Bio-Ethyl benzene, and Bio-Cyclohexane. Bio-Cumene can be used to produce phenol though a process involving cumene peroxidation. Bio-Ethylbenzene can be used as a precursor to produce biogenic styrene, polystyrene for use in a variety of products in food packaging, thermal insulation and appliances. Bio-Cyclohexane can be used as a precursor to produce cyclohexanone (Bio-Cyclohexanone) for use in manufacturing caprolactam, Nylon-6, which in turn can be used in films, wire coatings and food wraps.

Specific embodiments of the invention are described in the following examples, which do not limit the scope of the invention described in the claims.

### SPECIFIC EMBODIMENTS OF THE INVENTION

### Example 1

### Materials and Methods

Bacterial and Yeast Strains:

E. coli DH10B ElectroMAX cells are purchased from Invitrogen Life Technologies, Inc (Carlsbad, Calif.). E. coli BL21(DE3) cells are from Novagen (Madison, Wis.). S. cerevisiae strains are from Invitrogen (Brachmann C B, Davies A, Cost G J, Caputo E, Li J, Hieter P, Boeke J D (1988) Designer deletion strains derived from Saccharomyces cerevisiae S288C: a useful set of strains and plasmids for PCR-mediated gene disruption and other applications. Yeast 14(2):115-32).

Vector System:

The pESC Yeast Epitope Tagging Vector System (Stratagene, La Jolla, Calif.) is used to clone and express the Geranyl Pyrophosphate Synthase (gps) gene from tomato (Solanum lycopersicum) and the monoterpene synthase genes (α-pinene synthase from Pinus taeda (loblolly pine), γ-terpinene synthase from Citrus unshiu (satsuma) terpinolene synthase from Abies grandis (grand fir) d-limonene synthase from Citrus unshiu (satsuma) β-pinene synthase from Citrus limon (lemon) sabinene synthase from Salvia officinalis (garden sage)) into S. cerevisiae strains. The pESC vectors contain both the GAL1 and the GAL10 promoters on opposite strands, with two distinct multiple cloning sites, allowing for simultaneous expression of two genes. These promoters are repressed by glucose and induced by galactose. The pESC plasmids are shuttle vectors, allowing the initial construct to be made in E. coli (with the bla gene for selection on 100 μg/mL Ampicillin); however, no bacterial ribosome binding sites are present in the multiple cloning sites.

Reagents:

Polymerase chain reactions are carried out using an Opti-Prime PCR Optimization Kit (Stratagene) or Expand DNA polymerase (Roche Molecular Biochemicals; Indianapolis, Ind.). Plasmid DNA is purified from bacterial cells using a QIAprep Spin Miniprep kit (Qiagen, Valencia, Calif.), while plasmid DNA is purified from yeast cells with a Zymoprep Yeast Plasmid Miniprep kit (Zymo Research, Orange, Calif.). The Rapid DNA Ligation Kit is from Roche Diagnostics Corp (Indianapolis, Ind.). The QIAQuick Gel Purification and PCR Purification kits are purchased from Qiagen. The S.c. EasyComp™ Transformation Kit is from Invitrogen Corp (Carlsbad, Calif.). Microbial growth media components are from Becton Dickinson Microbiology Systems (Sparks, Md.) or VWR Scientific Products (So. Plainfield, N.J.), and other reagents are of analytical grade or the highest grade commercially available. Primers are purchased from Integrated DNA Technologies, Inc. Restriction enzymes are from New England Biolabs, Inc (Beverly, Mass.).

Equipments:

Electrophoresis of DNA samples is carried out using a Bio-Rad Mini-Sub Cell GT system (DNA) (Bio-Rad Laboratories, Hercules, Calif.), while protein samples are analyzed using a Bio-Rad Protein 3 mini-gel system and precast 4-15% gradient SDS-PAGE gels. An Eppendorf Mastercycler Gradient thermal cycler is used for PCR experiments. UV-visible spectrometry is done using a Molecular Devices SpectraMAX Plus spectrophotometer (Sunnyvale, Calif.). Electroporations of DNA samples are performed using a Bio-Rad Gene Pulser II system, while protein samples are analyzed using a Bio-Rad Protein 3 mini-gel system and precast 4-15% gradient SDS-PAGE gels. Automated DNA sequencing is carried by SeqWright (Houston, Tex.) using the dideoxynucleotide chain-termination DNA sequencing method.

Methods:

Recombinant DNA techniques for PCR, purification of DNA, ligations and transformations are carried out according to established procedures (Sambrook & Russell, 2001, MOLECULAR CLONING: A LABORATORY MANUAL, 3rd Edition, Cold Spring Harbor Laboratory Press, Cold Spring Harbor, N.Y.).

Recipes for Media:

LB Medium (Miller) contains (per liter): 10 g tryptone, 5 g yeast extract, and 10 g sodium chloride. Medium is autoclaved for 15 min at 121° C. For solid medium, 1.5% agar is added before autoclaving. 2×YT Medium contains (per liter): 16 g tryptone, 10 g yeast extract, and 5 g sodium chloride. Medium is autoclaved for 15 min at 121° C. SC-Leu Defined Medium (Per liter): 6.7 g Yeast Nitrogen Base without amino acids (Difco), 0.04 g adenine, 0.02 g uracil, 0.03 g lysine, 0.2 g threonine, 0.1 g aspartic acid, 0.02 g methionine, 0.05 g phenylalanine, 0.375 g serine, 0.03 g tyrosine, 0.04 g tryptophan, 0.02 g uracil, 0.02 g histidine, 0.1 g glutamic acid, 0.02 g arginine and 0.15 g valine. The mixture is autoclaved for 15 min at 121° C. and then cooled. SC-Ura Defined Medium (Per liter): 6.7 g Yeast Nitrogen Base without amino acids (Difco), 0.04 g adenine, 0.03 g lysine, 0.2 g threonine, 0.1 g aspartic acid, 0.06 g leucine, 0.02 g methionine, 0.05 g phenylalanine, 0.375 g serine, 0.03 g tyrosine, 0.04 g tryptophan, 0.02 g uracil, 0.02 g histidine, 0.1 g glutamic acid, 0.02 g arginine and 0.15 g valine. The mixture is autoclaved for 15 min at 121° C. and then cooled. SC-Ura-Leu Defined Medium (Per liter): 6.7 g Yeast Nitrogen Base without amino acids (Difco), 0.04 g adenine, 0.03 g lysine, 0.2 g threonine, 0.04 g tryptophan, 0.1 g aspartic acid, 0.02 g methionine, 0.05 g phenylalanine, 0.375 g serine, 0.03 g tyrosine, 0.02 g uracil, 0.02 g histidine, 0.1 g glutamic acid, 0.02 g arginine and 0.15 g valine. The mixture is autoclaved for 15 min at 121° C. and then cooled.

Isolation and Purification of monoterpenes in Fermentation Broth:

Cultures of S. cerevisiae transformants expressing the GPS and MS genes grown as described herein is subjected to centrifugation at 1500×g for 10 min at 4° C. The resulting aqueous supernatant and any visible oily layer (upper layer) containing monoterpenes (for example, γ-terpinene) is separated by decanting, siphoning, using a separatory funnel, or allowing the oily and aqueous fraction to separate in a buret and collecting the desired oily fraction. Once the monoterpene-containing oil is separated from the aqueous supernatant, the monoterpene (for example γ-terpinene) is purified by steam distillation at the boiling point of each monoterpene. The resulting distillate, the monoterpene of interest is collected. If the volume of oil is small, the oily fraction is separated from the aqueous supernatant by liquid-liquid extraction with an organic solvent. A small volume of a water-insoluble organic solvent such as methylene chloride or ether is added to the water-oil mixture, and the mixture is shaken. In this way, the oil is extracted into the organic solvent, which is then separated from the aqueous layer as described above (by decanting, siphoning or using a separatory funnel or buret). The organic solvent is removed by evaporation using a rota-yap leaving the monoterpene-containing oil. Samples containing monoterpenes such as any unprocessed culture supernatants and oily fractions are stored at 4° C. in hermetically sealed dark glass flask with rubber lids and covered with aluminium foil to protect the content from light.

Identification and Quantitation of the Monoterpene γ-Terpinene and ρ-Cymene:

The chemical composition of the monoterpene oils obtained, for example γ-terpinene, as well as their amounts, are determined using gas chromatography (GC) and gas chromatography-mass spectrometry (GC-MS) as described by Koba et al., J. Sci. Res. 1, 164-71 (2009). Commercially-obtained monoterpenes and ρ-cymene (BioMerieux Co., Paris, France) are used as standards to determine retention times and assist in quantification. The GC analysis is performed using a Varian 3300 type gas chromatograph equipped with FID detector. The columns are: (1) an apolar capillary column DB-5, with dimensions of 30 m×0.25 mm i.d. and film thickness of 0.25 μm; and (2) a polar column Supelcowax 10 having the same dimension and thickness. The operating conditions for the DB-5 column are: 50° C. for 5 min and then 50° C. to 250° C. at a rate of 2° C./min, while the operating conditions for the Supelcowax 10 column are: 50° C. for 5 min and then 50° C. to 200° C. at a rate of 2° C./min. The injector and detector temperatures are 250° C. and 300° C., respectively. The flow rate of the carrier gas helium is 1.50 ml/min. Samples of about 0.2 μL of non diluted oil are injected manually. The GC-MS analysis is performed using a Hewlett Packard 5890 SERIES II chromatograph, coupled with a mass spectrometer of the Hewlett Packard 5971 SERIES type operating in the El mode at 70 eV. The capillary column type is a DB5-MS column of dimension 30 m×0.25 mm i.d. with a film thickness of 0.25 μm. The amount of sample injected as well as GC/MS parameters are the same as that for the GC analysis. The oils are identified by their retention time, by their retention indices relative to C5-C18 n-alkanes, as well as by comparison of their mass spectra with authentic samples of each monoterpene and ρ-cymene. The percentage compositions of the monoterpenes are computed from the GC peak area.

Identification of Cymene Isomers:

GC-MS is performed in a Hewlett-Packard 5890/II gas chromatograph with an HP 5972A mass-selective detector, 30-m HP-5 MS quartz capillary column [30 m×0.25 mm, 0.25 μm stationary phase (diphenyl:dimethylsiloxane copolymer, 5:95), He carrier gas (1 mL/min), vaporizer temperature 280° C., column 50° C. (2 min)-10° C./min-280° C., ion source 173° C., interface between GC and MS detector 280° C., ionizing-electron energy 70 eV, data collection 1.2 scans/s for mass range 30-650 amu]. Preparative GC is performed by refurbishing for preparative work a Chrom-5 chromatograph with a flame-ionization detector and N₂ carrier gas. Products are separated using a steel column [4500×6 mm, 15% Apiezon L on Chromaton N-AW (0.250-0.315 mm), column temperature 120° C., vaporizer and detector 250° C.]. PMR and ¹³C NMR spectra of CDCl₃ solutions are recorded on a Bruker DRX-500 spectrometer (500.13 MHz for ¹H, 125.75 MHz, ¹³C) at 25° C. using solvent signals (δ_{c} 76.90 ppm, δ_{H} 7.24) as internal standards.

Conversion of Monoterpenes to ρ-Cymene:

Monoterpenes, for example, γ-terpinene obtained by S. cerevisiae fermentation, are converted to ρ-cymene by auto-oxidation, metal catalysis or with the assistance of a dehydrogenase or oxidase (for example, galactose oxidase). The monoterpene produced by S. cerevisiae fermentation is converted to ρ-cymene in an aromatization reaction by reverse hydrogenation using a skeletal catalyst such as Raney®-Nickel (Sigma-Aldrich) or by the action of a dehydrogenase or oxidase. The dehydrogenase or oxidase is expressed in the S. cerevisiae as described above or can be purchased directly. For example, galactose oxidase from Dactylium dendroides can be purchased in its purified and active form from Sigma-Aldrich (catalog #G7400-1KU). Galactose oxidase is reacted with a monoterpene, such as γ-terpinene according to the procedure of Taki et al., J Inorg Biochem 78:1-5 (2000), substituting terpinene for cyclohexadiene in the reaction to produce p-cymene. Detection, isolation and identification of the ρ-cymene product is described above.

### Example 2

### Cloning of Monoterpene Synthase and Geranylpyrophosphate Synthase Genes into Saccharomyces cerevisiae Strains

Construction of GPSpESCUra, and GPSpESCLeu:

The nucleic acid sequence of a geranyl pyrophosphate synthase (GP synthase or GPS) from Solanum lycopersicum is obtained from the National Center for Biotechnology Information (NCBI) Genbank (Accession number DQ286930). The GPS gene is cloned into the pESCUra and pESCLeu vectors singly behind the Gall promoter using the Bam HI and Xho I sites of Multiple Cloning Site 2. Primers for the synthesis of the gene with appropriate restriction sequences for the pESC vectors 5′ of the gene's ATG start codon and 3′ of each gene's stop codon are designed for PCR amplification using the Solanum lycopersicum (Tomato) cDNA library UC82-B (Vector: Lambda ZAP II vector, Average Insert Size: 1.0 kb, available from Agilent Technologies (Catalog #: 936004)) as template. The NH₃ terminus primer has a Bam HI restriction site (underlined) and the sequence 5′-GGCCGGATCCATGATATTTTCAAAGGGTTTAGC-3′ (SEQ ID NO: 1). The COOH terminus primer has the sequence 5′-GGCCCTCGAGCTATTTTGTTCTTGTGATGAC-3′ (SEQ ID NO: 2) (Xho I restriction site is underlined). The start and stop codons are bolded. The gene encoding GPS is amplified by PCR using the primers with Bam HI and Xho I restriction sites and the S. lycopersicum cDNA library as template. The thermocycler program has a hot start at 96° C. for 5 min and 30 repetitions of the following steps: 94° C. for 30 sec, 55° C. for 1 min, and 72° C. for 1 min and 30 sec. After the 30 cycles, the sample is incubated at 72° C. for 7 min and then stored at 4° C. The PCR product is purified from a 1% TAE-agarose gel (QIAQuick Gel Purification kit), and after restriction digestion of both the PCR product and the pESCUra vector with Bam HI and Xho I, the ligation is carried out using the Rapid DNA Ligation Kit (Roche). The ligation mixture is desalted and transformed into E. coli DH 10B ElectroMAX cells using the BioRad recommended procedure for transformation of E. coli cells with 0.2 cm micro-electroporation cuvettes. After recovery in SOC medium, the transformation mixture is plated on LB plates containing 100 μg/mL of ampicillin. Plasmid DNA is isolated and purified from liquid cultures [5 mL 2×YT medium+ampicillin (100 μg/mL) grown overnight at 37° C.] of colonies picked from the LB+ampicillin (100 μg/mL) plates. Plasmids are screened by restriction digestion, and the sequences verified by dideoxynucleotide chain-termination DNA sequencing.

The plasmid DNA from a pESCUra clone carrying the S. lycopersicum GPS, as well as plasmid pESCLeu, are digested with Bam HI and Xho I. The 1.3 Kbp band carrying the GPS gene and the linear pESCLeu plasmids are purified from a 1% TAE-agarose gel and ligated as described above. After removing the salts and proteins using a QIAQuick PCR Clean-up kit, the ligation mixtures are transformed into E. coli DH10B cells. Plasmid DNA is purified from ampicillin resistant cells and screened by restriction digestion.

Construction of GPS/Monoterpene Synthase pESUra, and GPS/Monoterpene SynthasepESCLeu:

The monoterpene synthase (MS) genes are amplified by PCR using primers with Spe I and Pac I restriction sites. Primers for amplifying the MS genes are shown below with restriction enzyme recognition sites (Spe I on the NH₃ termini; Pac I on the COOH termini) underlined. The ATC start codons, as well as the various stop codons are shown in bold.

α-pinene synthase (α-PS) from Pinus taeda (lob-
lolly pine)(GenBank: AF543530)
NH₃ terminus:
(SEQ ID NO: 3)
5′-AATTACTAGTATGGCTCTGGTTTCTGCTGTCC-3′
COOH terminus:
(SEQ ID NO: 4)
5′-GGTTAATTAACTACAAAGGCACAGTTTCAACCAC-3′
γ-terpinene synthase (γ-TS) from Citrus unshiu
(satsuma) Gen Bank: AB110640)
NH₃ terminus:
(SEQ ID NO: 5)
5′-GGCCACTAGTATGGCTCTTAATCTGCTATCTTC-3′
COOH terminus:
(SEQ ID NO: 6)
5′-GGTTAATTAATTAAGGAATGGGATCAATAAATAAAGA-3′
terpinolene synthase (TS) from Abies grandis
(grand fir)(GenBank: AF139206)
NH₃ terminus:
(SEQ ID NO: 7)
5′-GGCCACTAGTATGGCTCTTGTTTCTATCTTG-3′
COOH terminus:
(SEQ ID NO: 8)
5′-GGTTAATTAATTACAAAGGCACAGACTCAAGG-3′
d-limonene synthase (d-LS) from Citrus unshiu
(satsuma)(GenBank: AB110637)
NH₃ terminus:
(SEQ ID NO: 9)
5′-GGCCACTAGTATGTCTTCTTGCATTAATCCCTC-3′
COOH terminus:
(SEQ ID NO: 10)
5′-GGTTAATTAATCAGCCTTTGGTGCCAGGAGATG-3′
β-pinene synthase (β-PS)from Citrus limon (lemon)
(GenBank: AF514288)
NH₃ terminus:
(SEQ ID NO: 11)
5′-AATTACTAGTATGGCTCTTAATCTGCTCTC-3′
COOH terminus:
(SEQ ID NO: 12)
5′-GGTTAATTAATTAAGCAATGGGATCAAAAAATAAGG-3′
sabinene synthase(SS) from Salvia officinalis
(garden sage)(GenBank: AF051901)
NH₃ terminus:
(SEQ ID NO: 13)
5′-GGCCACTAGTATGTCTTCCATTAGCATAAACATA-3′
COOH terminus:
(SEQ ID NO: 14)
5′-GGTTAATTAATCAGACATAAGGCTGGAATAGCAG-3′

To make cDNA libraries of Salvia officinali, Citrus limon, Citrus unshiu, Abies grandis and Pinus taeda, fresh leaf tissue is harvested and total RNA extracted using the Qiagen RNeasy plant mini kit (Catalog #79403) following the manufacturer's instructions. Messenger RNA is extracted from total RNA using the Invitrogen FastTrack MAG Micro mRNA isolation kit (Catalog #K1580-01). From mRNA, the cDNA library is constructed using the Invitrogen SuperScript Full Length cDNA library construction kit (Catalog #A11181). Alternately, the gene sequences can be directly synthesized using Overlap Extension PCR (Stemmer, W. P., Crameri, A., Ha, K. D., Brennan, T. M. and Heyneker, H. L. (1995) Single-step assembly of a gene and entire plasmid from large numbers of oligodeoxyribonucleotides. Gene, 164, 49-53). The PCR products (all approximately 1.8 to 1.9 kbp) are purified from 1% TAE-agarose gels (QIAQuick Gel Purification Kit), and the sequences verified by dideoxynucleotide chain-termination DNA sequencing. GPSpESCUra and GPSpESCLeu plasmids and the PCR products are digested with Spe I and Pac I. The plasmids are purified from a 1% TAE-agarose gel, while the restriction digest mixtures of the PCR products are purified using a QIAQuick PCR Clean-up kit. Ligations and transformations into E. coli DH10B cells are carried out as described above. Plasmid DNA are purified from ampicillin resistant cells and screened by restriction digestion. In total, the following 12 plasmid constructs (6 sets of 2 plasmids each) are generated. SET 1 includes GPS/α-PSpESCLeu (geranylpyrophosphate synthase/alpha-pinene synthase on pESCLeu vector) and GPS/α-PSpESCUra (geranylpyrophosphate synthase/alpha-pinene synthase on pESCUra vector). SET 2 includes GPS/γ-TSpESCLeu (geranylpyrophosphate synthase/gamma-terpinene synthase on pESCLeu vector) and GPS/γ-TSpESCUra (geranylpyrophosphate synthase/gamma-terpinene synthase on pESCUra vector). SET 3 includes GPS/TSpESCLeu (geranylpyrophosphate synthase/terpinolene synthase on pESCLeu vector) and GPS/TSpESCUra (geranylpyrophosphate synthase/terpinolene synthase on pESCUra vector). SET 4 includes PS/d-LSpESCLeu (geranylpyrophosphate synthase/d-limonene synthase on pESCLeu vector) and GPS/d-LSpESCUra (geranylpyrophosphate synthase/d-limonene synthase on pESCUra vector). SET 5 includes GPS/β-PSpESCLeu (geranylpyrophosphate synthase/beta-pinene synthase on pESCLeu vector) and GPS/β-PSpESCUra (geranylpyrophosphate synthase/beta-pinene synthase on pESCUra vector). SET 6 includes GPS/SSpESCLeu (geranylpyrophosphate synthase/sabinene synthase on pESCLeu vector) and GPS/SSpESCUra (geranylpyrophosphate synthase/sabinene synthase on pESCUra vector). Plasmids carrying copies of the GPS gene and the MS genes are chosen for transformation into the S. cerevisiae strains. Competent cells of the following S. cerevisiae strains are prepared using an S.c. EasyComp™Transformation Kit (Invitrogen Corp). Aliquots (50 A) were frozen at −80° C. and thawed just prior to use. The S. cerevisiae strains are: (1) BY4743 diploid parental strain (MATa/α, his3Δ1/his3Δ1, leu2-Δ0/leu2-Δ0, met15-Δ0/MET15⁺, LYS2⁺/lys2-Δ0, ura3-Δ0/ura3-Δ0); and (2) Y22884: derived from BY4743 diploid parental strain (Mat a/α, his3Δ1/his3Δ1, leu2Δ0/leu2Δ0, lys2Δ0/LYS2, MET15/met15Δ0, ura3Δ0/ura3Δ0, YHR190w::kanMX4/YHR190w). Transformations of the pESC vector constructs into S. cerevisiae competent cells are also carried out using the S.c. EasyCompr™Transformation Kit. The vectors pESCLeu or the 6 versions of GPS/Monoterpene SynthasepESCLeu are transformed singly into each of the two S. cerevisiae strains. A 100 μL aliquot from each transformation reaction was spread on SC-Leu plates (medium recipes from Stratagene pESC manual). The plate medium of the Y22884 strains also contains 0.2 mg/mL geneticin. The plates are incubated for 2 days at 30° C. Colonies from each plate are used to inoculate 5 mL liquid cultures of SC-Leu medium. The cultures are incubated overnight at 30° C., and the cells are harvested by centrifugation. Plasmid DNA is isolated from the cells using a Zymoprep Yeast Plasmid Miniprep kit. After analysis of the isolated DNA by PCR, one isolate from each construct that generated the predicted PCR products is chosen for expression studies.

The two vectors pESCLeu and pESCUra, or the two vectors of each of Sets 1-6 (GPS/MSpESCLeu and GPS/MSpESCUra), are simultaneously transformed into each S. cerevisiae strain as described above, and the transformation mixtures are plated on SC-Ura-Leu plates. The plate medium of the Y22884 strains also contains 0.2 mg/mL geneticin. After analysis by PCR, an isolate that carries the multiple plasmids without the GPS/MS inserts and isolates that carry plasmids with the GPS/MS genes inserted downstream of the GAL1 and GAL10 promoters (respectively) are chosen for expression studies.

### Example 3

### Overexpression of GPS and MS Genes in S. cerevisiae Strains and Accumulation of Monoterpenes in the Fermentation Broth

Induction of the GPS and MS Genes:

S. cerevisiae strains carrying the pESCLeu plasmid with or without the GPS/MS inserts are grown in 5 mL SC-Leu containing 2% glucose overnight at 30° C. with shaking. One mL from each culture is transferred to 5 mL of SC-Leu medium containing 1% raffinose and 1% glucose and the incubation is continued for 10 hours. The medium of the Y22884 strains also contains 0.2 mg/mL geneticin. The OD₆₀₀ of each culture is determined and the amount of culture necessary to obtain an OD₆₀₀ of 0.16 to 0.4 in 100 mL of SC-Leu containing 1% galactose and 1% raffinose (induction medium) is calculated. The calculated volume of cells is centrifuged at 1500×g for 10 min at 4° C., and the pellet is resuspended in 100 mL induction medium. Each construct is grown at 30° C. with shaking at 250 rpm from 0 to 90 hours.

Determination of Monoterpene Formation:

At 0, 13, 24, 48, 66 and 90 hours (h), aliquots of fermentation broth are removed from transformants carrying the vector alone and from transformants carrying the vector with the GPS/MS inserts. Their OD₆₀₀ are measured. Product formation is determined in the samples using the methods described in the Materials and Methods Section above. Briefly, aliquots are centrifuged to remove to cells and to separate the aqueous fermentation broth and any oily fraction that may be present. When fermentation broth samples from BY4743 and Y22884 transformed with GPS/MS constructs are centrifuged, a cell pellet, an aqueous fraction above it, and an oily fraction are obtained. As a result, γ-terpinene present in the oily fraction above the aqueous supernatant is recovered. When fermentation broth samples from BY4743 and Y22884 transformed with the parental pESC Leu (and pESCUra) constructs are centrifuged, a cell pellet, an aqueous fraction above it, but no oily fraction are obtained. As a result, monoterpenes present in the oily fraction above the aqueous supernatant is recovered. The individual monoterpenes in the oily fraction are further purified by distillation at their characteristic boiling points as follows: γ-terpinene at 182° C.; d-Limonene at 176° C.; α-Pinene at 155° C.; β-Pinene at 165° C.; Terpinolene at 185° C.; and Sabinene at 163° C.

### Example 4

### Overexpression of GPS & MS Genes & Accumulation Monoterpenes in Fermentation Broth Using S. cerevisae Constructs Carrying Multiple Copies of the Gene

Induction of the GPS and MS Genes:

S. cerevisiae strains transformed using the 2 plasmids (pESCLeu, and pESCUra) or each of the 6 sets of plasmids (Sets 1-6, described above) are grown in 5 mL SC-Leu-Ura containing 2% glucose overnight at 30° C. with shaking. One mL from each culture is transferred to 5 mL of SC-Ura-Leu medium containing 1% raffinose and 1% glucose, and the incubation is continued for 9 h. The medium of the Y22884 strains also contains 0.2 mg/mL geneticin. The OD₆₀₀ of each culture is determined, and the amount of culture necessary to obtain an OD₆₀₀ of 0.2 to 0.4 in 50 mL of SC-Ura-Leu containing 1% galactose and 1% raffinose (induction medium) is calculated. The calculated volume of cells is centrifuged at 1500×g for 10 min at 4° C., and the pellet is resuspended in 50 mL induction medium. Each construct was grown at 30° C. with shaking at 250 rpm from 0 to 36 h.

Determination of Monoterpene Formation and Isolating of Monoterpene:

At 0, 11.5, 16, 21, and 35 hours, aliquots of fermentation broth are removed, and the OD₆₀₀ is measured. Product formation is determined using the methods described in the Materials and Methods section above. Briefly, the aliquots are centrifuged to separate the cells, the aqueous supernatant and any oily fraction containing monoterpene. Centrifugation of the fermentation broth samples from transformed constructs of BY4743 and Y22884 results in a cell pellet, an aqueous supernatant and the monoterpene-containing oily fraction. Little or no product is detected in the fermentation broth of the strains carrying only the pESC vectors.

### Example 5

### Isolation of Monoterpenes from Citrus Rinds by Steam Distillation or Solvent Extraction

To isolate monoterpenes by steam distillation, about 150 g of orange or lemon peels are chopped into pieces about the size of a grain of rice and then transferred to a 500 mL round bottom flask containing 150 mL of distilled water. A distillation apparatus is set up for steam distillation by attaching the round bottom flask containing the peels and water to a distillation head with thermometer, condenser, adaptor and distillate receiving flask. The distillation flask is heated with a heating mantle, the heat supply is adjusted to distill at a rate of about one drop per second. About 70 mL of distillate is collected and transferred to a buret where the liquid layers settle and separate into a bottom layer of water and a top layer of oil. Trapped air or oil bubbles are dislodged using a copper wire with a loop. Once the liquid layers have separated, the stopcock on the buret is opened and the bottom water layer is drain into an Erlenmeyer flask at no more than one or two drops per second. The oil layer is collected in a vial. A sprinkling of granular anhydrous sodium sulfate, a drying agent, is added to the oil to remove any water remaining. About 0.2 μL to about 3 μL of citrus oil is analysed by gas chromatography (GC) (SF-96 column at 150° C.). To isolate monoterpenes from citrus rinds by solvent extraction, the flavedo portion of citrus fruit rind (i.e. the color portion of the peel) is finely grated using a cheese grater. About 2.5 g of the finely grated peel is transferred to a separating funnel, extracted three times, each using about 7 mL of pentane for 10-minute intervals with frequent venting during extraction. The three extracts are combined and dried for 15 minutes over anhydrous sodium sulfate, which is then removed by filtration. The resulting extract is transferred to a tared 50 mL beaker, where it is warmed over low heat (at about 35° C.) under a gentle stream of nitrogen to remove pentane while minimizing evaporation of the volatile components of the citrus essential oils. The essential oil containing monoterpenes is obtained when pentane is evaporated.

### Example 6

### Conversion of Limonene to Cymene by Dehydrogenation

Limonene is converted to cymene using a palladium catalyst on charcoal as described by See Roberge et al., Catalytic Aspects in the Transformation of Pinenes to ρ-Cymene, Applied Catalysis A: General 215: 111-124 (2001). About 20 mL (or 0.125 mol) of limonene is transferred to a 3-necked 100 mL round bottomed flask fitted with a condenser with an oil trap. One neck is fitted with a suba-seal through which nitrogen gas is passed through a syringe needle. The third neck is fitted with a glass stopper through which samples are taken. The limonene is heated with magnetic stirring to 100° C. Prior to catalyst addition, about 2 mL of limonene is withdrawn using a glass pipette, and paladium (Pd) on charcoal (0.1 g of 5% wt) is added to the flask through the side arm via a glass funnel. Any residue remaining on the glass funnel is washed into the flask with the 2 mL of limonene previously withdrawn. Reaction is performed under a nitrogen atmosphere at 100° C. for 3 hours. At the end of reaction, the reaction mixture is cooled, filtered and weighed. A sample of the filtrate is analysed by GC using a DB17 column (Injector Temperature: 200° C.; Detector Temperature: 200° C.; Initial Temperature: 60° C.; Final Temperature: 200° C.; Ramp rate: 8° C./minute; Injection: 1 AL

Limonene is converted to cymene by oxidative dehydrogenation using H₃-[PMo₁₂O₄₀] as described by Neumann & Lissel, Aromatization of hydrocarbons by Oxidative Dehydrogenation Catalyzed by the Mixed Addenda Heteropoly Acid H₃PMo₁₀V₂O₄₀, Journal of Organic Chemistry 54: 4607-10 (1989). Briefly, about 200 mg (0.24 mmol) of H₃-[PMo₁₂O₄₀].aq is dissolved in 20 mL of 1,2-dichloroethane (DCE) by addition of tetraglyme (2.5 mmol, 550 μL) in a small 50 mL beaker with magnetic stirring. The catalyst is filtered into the reaction vessel through the side arm of a two necked 100 mL round bottomed flask. About 2.72 g (ca. 20 mmol) of D-limonene (Sigma-Aldrich) is added through the side arm, which is then stoppered. The reaction is heated to 70° C. with constant magnetic stirring while exposed to the air through an unstoppered condenser. After about 4 hours, the reaction flask is cooled to room temperature and the products are decanted into a 100 mL separating funnel. The catalyst is extracted three times, each with 20 mL portions of distilled water, while the organic phase is decanted into a conical flask containing a drying agent, e.g. magnesium sulfate, and allowed to dry for at least 15 minutes. The product mixture is then filtered into a clean flask, and GC analysis is performed as described above.

Limonene is converted to cymene under “solvent free” conditions over mesoporous silica-alumina supports heated by microwave irradiation as described by Martin-Luengo et al., Synthesis of ρ-Cymene from Limonene, a Renewable Feedstock, Applied Catalysis B: Environmental 81: 218-24 (2008). Briefly, the catalytic support is prepared by the procedure described by Meyer, German Patent, DE 38,39,580 C1 (1990), which is based on the acid hydrolysis of aluminium hexylate dissolved in hexanol (6 weight %) and orthosilicic acid (3 weight %) mixtures. The resulting gel (pseudo-boehmite) is dried with pressurized air and then heat-treated at 180° C. for 5 hours with autogenous pressure. Silica contents ranging from 1 to 40 weight %, designated as SIRAL 1 to SIRAL 40, are used.

D-limonene (Sigma-Aldrich) (99.9%) is used in the reactions without further purification. The reactant and product mixtures are extracted using ethanol.

A focalised monomodal system type microwave oven, e.g. Synthewave 402 from Prolabo, is used for the catalytic reaction, which is performed under both dry media and reflux conditions. For the dry media reactions 50 mL of limonene are physically mixed with 200 mg of solid, and the mixture is placed in a glass reactor and irradiated at maximum power output of 300 W for fixed periods of time: 5, 10 or 20 min. When the reaction is carried out in reflux conditions 500 mg of solid are mixed with 5 mL of limonene, and the mixture heated to a maximum temperature of 165° C. for 10 or 20 min with the power output of the microwave oven controlled automatically to avoid overheating of the reaction mixture. The final temperature chosen is slightly lower than the boiling points of the reactant and products (limonene 175° C., p-cymene 177° C.) in order to control the reaction. A reflux column is used to ensure that there is no loss of materials. At the end of the experiments, the reaction mixtures are cooled and the reactants and products extracted by dissolution in ethanol. These mixtures are analysed by GC-MS (Hewlett Packard 5890 series II GC with a 25 m methyl silicone capillary column heated in a helium flow from 50 to 170° C. at 6° C. min⁻¹, coupled to a Hewlett Packard series 5971 mass spectrometer). To ensure the reproducibility of the results the injector and detector were heated to 180 and 250° C., respectively, to avoid condensation of the mixtures. Following the extraction of the reaction products with ethanol, the catalytic activities of the samples are redetermined using the same protocols as described above. The above procedure converts limonene to α- and γ-terpinene, γ-terpinolene and ρ-cymene.

### Example 7

### Conversion of α-Terpinene to Cymene by Dehydrogenation

α-Terpinene is converted to cymene by isomerization on sulfated zirconia according to the method described by Comelli et al., Isomerization of α-Pinene, Limonene, α-Terpinene and Terpinolene on Sulfated Zirconia, Journal of the American Oil Chemists' Society 82:531-35 (2005). Briefly, the transformation of α-terpinene is performed in a batch reactor, at constant temperature, with magnetic stirring. The catalyst is prepared by impregnating zirconium hydroxide with a solution of H₂SO₄ (1 N; Merck, Darmstadt, Germany) in methanol (Carlo Erba, Milano, Italy). Zirconium hydroxide is obtained by hydrolysis of zirconyl chloride (ZrOCl₂.6H₂O; Fluka, Buchs, Switzerland). The nominal concentration of H₂SO₄ in the catalyst is 15%. The precursor is calcined up to 600° C. for 4 hours before use in the reaction. The surface area of the support and catalysts and the distribution of pore sizes are determined using the N₂ BET method in a Micromeritics Accusorb 2100E instrument. The crystalline structure of catalysts is determined by X-ray diffraction (XRD) studies on Rigaku D-Max III equipment with Cu Kα radiation (λ=1.5378 Å, 40 K, 30 mA). The 2θ range analyzed is between 5° and 70°. FTIR spectra of the catalysts are obtained in a Bruker IFS66 FTIR instrument using KBr pellets. FTIR spectra of catalysts with adsorbed ammonia are used to determine the presence of Brönsted acidity. The adsorption is carried out at room temperature by passing pure ammonia (15 cm³/min) for 30 minutes. Excess ammonia is eliminated by applying vacuum for 12 hours.

The analysis of the reaction components is performed by GLC with a capillary column DB1 (Supelco, Bellefonte, Pa.) of 60 m and the temperature is increased from 75 up to 200° C. at a rate 3° C./min. The identification of products is made by comparison of retention times with terpene standards and confirmed by GC-MS. Under the above conditions, α-terpinene can be converted to ρ-cymene by isomerization at 120° C.

### Example 8

### Conversion of Cineole to Cymene

Leita et al. describes the hydrogenation of cineole to ρ-cymene. See Leita et al., Production of ρ-Cymene and Hydrogen from a Bio-renewable Feedstock-1,8-Cineole (Eucalyptus Oil), Green Chemistry 12: 70 (2010). Briefly, high surface area γ-Al₂O₃ pellets (200 m²g⁻¹) are used as a slightly acidic catalyst and as a solid support for molybdenum iron, cobalt, chromium and palladium metals. The metal-doped γ-Al₂O₃ catalysts are prepared by a wet impregnation technique using 1 M aqueous solutions of the appropriate metal salts. About 100 mL of 1 M metal nitrate solution is poured over 70 g of γ-Al₂O₃ pellets (Saint-Gobain N or Pro, USA) heated in a vacuum oven at 90° C. overnight. The mixture is stirred with a spatula and left to stand at room temperature overnight. The resultant metal-impregnated γ-Al₂O₃ pellets are collected, washed three times with deionized water and dried in a vacuum oven at 90° C. overnight. The coated pellets are transferred to a crucible and calcined in air at 350° C. for 12 hours. As controls, undoped γ-Al₂O₃ pellets are subjected to the same treatment as the metal doped samples before use, and glass beads are used as a blank reaction surface.

The vapour phase catalytic conversion of cineole is performed using an electrically heated tubular down-flow reactor (13.5 mm internal diameter, 300 mm length) with the catalyst held as a fixed bed at atmospheric pressure. A K-type thermocouple is used to monitor the temperature of the bed. All thermocouples, furnaces, heating bands and mass flow controllers (MFC) are controlled. The liquid product is collected at 40° C. in a stainless steel trap, while the gaseous products are sent through a second trap at 0° C. to an online gas chromatograph (GC).

About 3 g of catalyst is loaded into a stainless steel mesh basket placed inside the tubular reactor. The furnace is set to an initial temperature of 250° C. and allowed equilibrate for one hour. Cineole is injected upstream of the pre-heater at a rate of 0.5 mL min⁻¹ with an ISCO 500D syringe pump. The carrier gas is a blended mixture of the amount of oxygen required in argon containing a 5.1% helium internal standard fed at a constant rate of 150 mL min⁻¹. Once at equilibrium, gas samples are taken and liquid products collected. The furnace temperature is raised by 50° C. and the procedure is repeated until the final reaction temperature of 500° C. is reached.

The liquid product obtained consists of an oily, hydrophobic phase and an aqueous phase. The analysis of liquid products is performed using a GC-MS fitted with an auto sampler. About 10 mL of the hydrophobic phase is dissolved in 1.5 mL of acetonitrile (Aldrich) doped with 0.1% mesitylene (Aldrich) as an internal standard. Chromatographic standards of 1,8-cineole, ρ-cymene and dipentene are prepared using the same sample method. Major products are characterized by ¹H and ¹³C NMR. The yield of ρ-cymene is defined as the percentage of ρ-cymene in the whole hydrophobic phase. Selectivity for ρ-cymene is defined as the percentage of ρ-cymene in the non-cineole fraction of the hydrophobic phase. Results indicate that the non-cineole fraction of the hydrophobic phase includes ρ-cymene and dipentene as major products.

Conversion of cineole to ρ-cymene is catalyzed by undoped γ-Al₂O₃ pellets at 0, 7.3 and 14.6% oxygen and about 400° C. as well as by chromium-doped γ-Al₂O₃ pellets at 14.6% oxygen and ˜400° C. In addition ρ-cymene is the major product when molybdenum-doped γ-Al₂O₃ catalyst and palladium-doped γ-Al₂O₃ are used as catalysts at 0 and 7.3% oxygen and ˜390° C.

### Example 9

### Transalkylation of Cymene and Benzene to Produce Cumene and Toluene

Renewable cumene and toluene are produced by the transalkylation of benzene with renewable cymene according to the method described by Innes et al., Liquid Phase Alkylation or Transalkylation Process Using Zeolite Beta, U.S. Pat. No. 4,891,458. To prepare the zeolite beta, about 524.06 g of Ludox AS-30 silica solution is added dropwise to 20.85 g of sodium aluminate Na₂Al₂O₄.3H₂O and 652.22 g of 20% tetraethylammonium hydroxide solution. The mixture is stirred for two days without heat and then charged to a one-gallon autoclave. A large amount of crystalline material is formed after six days in the autoclave at 150° C. without stirring. The material is filtered, washed, and oven-dried overnight at 100° C. to yield 141 g of crystalline powder, which is then calcined for 10 hours at 538° C. (1000° F.).

Three 118 g amounts of calcined zeolite beta are subjected to ammonium exchanged four times. The exchanges are performed by soaking the zeolite in approximately two liters of a 0.7 N ammonium nitrate solution overnight at 100° C. (212° F.). After each of the first three exchanges, the supernatant liquids are decanted and fresh ammonium nitrate solution added. After the final exchange, the product is filtered, washed with distilled water, and oven-dried.

The ammonium-exchanged zeolite beta is calcined for five hours at 538° C. (1000° F.) to convert the zeolite to its hydrogen form. A 320 g portion of the calcined zeolite is dry-mixed with 112.7 g of Catapal alumina (71% Al₂O₃). Distilled water and dilute nitric acid are added to peptize the alumina and bring the mixture to a consistency suitable for extrusion. A hydraulic press is used to extrude the mixture through a 1/16-inch die, and the extrudates are collected in a large evaporating dish, oven-dried, and calcined at 204-538° C. (400-1000° F.).

The transalkylation feedstock is prepared by blending benzene with renewable cymene in a four to one (benzene to cymene) weight ratio. The feedstock is reacted over steam stabilized Y (Linde LZ-Y82), omega (Linde ELZ-6), rare earth Y (Linde SK-500), or zeolite beta catalysts at 4.3 WHSV, 163° C. (325° F.) and 600 psig.

For the reaction, each of the catalysts is obtained as 1/16 extrudates with a sodium content less than 200 ppm. The zeolite beta catalyst is prepared from 80% zeolite beta (Si/Al=13) and 20% alumina binder. The extrudates are broken into pieces about ⅛″, calcined, and charged to a reactor as follows. The bottom portion of a ⅜-inch diameter tubular reactor is filled with quartz granules to act as preheat zone. Two to three grams of catalyst, depending on zeolite content, is charged on top of the quartz bed. Dry nitrogen is flowed over the catalyst at atmospheric pressure, while the reactor is heated to 163° C. (325° F.) by a three-zone electric furnace. The temperature is measured by a sheathed thermocouple positioned just above the top of the catalyst bed. The control valve is closed and the unit pressurized to 600 psig. The nitrogen is then turned off and the feedstock flow started at 12 mL/h. Pressure is maintained by diluting the reactor effluent with sufficient nitrogen to vaporize all the reaction products and passing the combined stream through the heated control valve. The vaporized product is analyzed by an on-stream gas chromatograph. The reaction is conducted at 4.3 WHSV (grams of benzene and cymene feed per gram of catalyst per hour), 163° C. (325° F.) and 600 psig.

All four reactions yield cumene and toluene; the reactions catalyzed by zeolite beta and steam stabilized Y zeolite yielding the greatest amounts of products.

### Example 10

### Transalkylation of Cymene and Benzene to Produce Cumene and Toluene

Renewable cumene and toluene are produced by the transalkylation of benzene with renewable cymene according to the method described by Hanai et al., Migration of Alkyl Groups of p-Cymene, Chiba Daigaku Bunrigakubu Kiyo Shizen Kagaku 5: 53-5 (1967). For the transalkylation, the benzene is prepared by agitation with concentrated sulfuric acid to remove thiophene, dried using calcium chloride, and purified by fractional distillation using a 1 m long separation column packed with 5 mm diameter Fenske helices. Cymene is prepared according to the method of Le Fevre et al. (J. Chem. Soc. 480 (1935)) and then purified by distillation using a concentric type rectification column (80 theorectical stages). No. 1 reagent grade aluminum chloride is grinded in a mortar prior to use. The transalkylation is performed as follows. About 70 mmol of ρ-cymene and 630 mmol of benzene are placed in a three-necked round bottom flask. A ground glass joint-type agitator is installed in the central neck, a Dimroth condenser equipped with a calcium chloride tube is installed in a side neck, and a bent-tube sample bottle loaded with 14 mmol of anhydrous aluminum chloride is installed in the other side neck. The assembly is placed in a constant temperature bath set at 60+/−0.5° C. When the reaction solution reached this temperature, the sample tube is rotated to add the anhydrous aluminum to the reaction solution, and the reaction is carried out for 45 minutes. After completion of the reaction, about 100 mL of water is added, followed by 5% aqueous sodium hydroxide solution. After the aluminum salts dissolves, the mixture is extracted three times using ether and then dried over anhydrous sodium sulfate. Gas chromatography (GC) is used to detect and identify the reaction products and chlorobenzene is used as an internal standard. For GC analysis, the equipment used and reaction conditions are as follows: (1) Support is DIASOLID M, 40-60 mesh; (2) Fixed phase liquid is 10% dioctyl sebacate; (3) Column length is 2 m; (4) Carrier gas is helium; (5) Carrier gas flow rate is 60 mL/minute; and (6) Glass temperature is 80° C.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A process for producing a renewable aromatic compound comprising (a) contacting a renewable cyclic monoterpene with a dehydrogenation catalyst under conditions effective for the oxidation of the cyclic monoterpene to renewable ρ-cymene and H₂, and (b) contacting the renewable ρ-cymene with benzene and a transalkylation catalyst under conditions effective for the transalkylation of the benzene with the renewable ρ-cymene to produce renewable cumene and renewable toluene.

2-6. (canceled)

7. The process of claim 195, wherein the cell over expresses at least one gene in the mevalonic acid pathway, non-mevalonic acid pathway, or mevalonic acid and non-mevalonic acid pathways.

8. (canceled)

9. The process of claim 195, wherein the cell over expresses a gene encoding HMG-CoA reductase or deoxy-xylulose phosphate synthase.

10-12. (canceled)

13. The process of claim 195, wherein the monoterpene synthase is a limonene synthase, α-pinene synthase, β-pinene synthase, terpinene synthase, terpinolene synthase or sabinene synthase.

14-16. (canceled)

17. A process for producing a renewable aromatic compound comprising contacting benzene with cymene and a transalkylation catalyst under conditions effective for the transalkylation of benzene with cymene to produce cumene and toluene, wherein the benzene comprises renewable benzene wherein all the carbons are renewable carbons, and the cymene comprises renewable cymene wherein all the carbons are renewable carbons.

18. A process for producing a renewable aromatic compound comprising:
(a) Contacting non-renewable benzene with renewable cymene and a first transalkylation catalyst under conditions effective for the transalkylation of the benzene with the cymene to produce renewable cumene and renewable toluene;
(b) Isolating at least a portion of the renewable toluene;
(c) Contacting the renewable toluene with H₂ under conditions effective to produce renewable benzene;
(d) Isolating at least a portion of the renewable benzene; and
(e) Contacting the renewable benzene with renewable cymene and a second transalkylation catalyst under conditions effective for the transalkylation of the renewable benzene with the renewable cymene to produce renewable cumene and renewable toluene.

19. The process of claim 18, wherein the renewable benzene of step (d) is combined with the non-renewable benzene of step (a) prior to contact with renewable cymene and the transalkylation catalyst.

20-25. (canceled)

26. The process of claim 1, wherein the cyclic monoterpene is limonene, terpinene, pinene, terpinolene, sabinene or cineole.

27-34. (canceled)

35. The process of claim 1, wherein the cyclic monoterpene is isolated from citrus rind or citrus processing wastes.

36-38. (canceled)

39. The process of claim 1, wherein the benzene comprises renewable benzene.

40. The process of claim 39, wherein the benzene further comprises non-renewable benzene.

41-48. (canceled)

49. The process of claim 1, wherein the transalkylation catalyst is a zeolite of the faujasite structure in the hydrogen form, a hydrogen mordenite, or another hydrogen zeolite with pore diameter of about 5.2 to about 7.8 Angstrom.

50-55. (canceled)

56. The process of claim 1, wherein the dehydrogenation catalyst is a dehydrogenase, oxidase or a metal catalyst.

57-64. (canceled)

65. The process of claim 1, further comprising isolating at least a portion of the renewable toluene or renewable cumene from the transalkylation product mixture.

66-73. (canceled)

74. The process of claim 65, wherein the renewable toluene is contacted with a transalkylation catalyst under conditions effective to produce renewable xylenes and renewable benzene by toluene disproportionation.

75.-82. (canceled)

83. The process of claim 74, further comprising recovering at least a portion of the renewable xylenes.

84. The process of claim 83, further comprising separating the renewable xylenes to m-xylene, o-xylene and ρ-xylene.

85-86. (canceled)

87. The process of claim 84, further comprising contacting the renewable ρ-xylene with O₂ in the presence of a catalyst under conditions effective for the oxidation of ρ-xylene to renewable terephthalic acid.

88-194. (canceled)

195. The process of claim 1, further comprising cultivating a cell that comprises a monoterpene synthase under conditions effective to produce a cyclic monoterpene and isolating at least a portion of the cyclic monoterpene prior to contacting the cyclic monoterpene with the dehydrogenation catalyst under conditions effective for the oxidation of the cyclic monoterpene to renewable ρ-cymene as in step (a), then isolating a portion of the ρ-cymene and contacting the ρ-cymene with benzene and a transalkylation catalyst under conditions effective for the transalkylation of the benzene with the renewable ρ-cymene as in step (b).

196. The process of claim 1, wherein steps (a) and (b) are performed by a process comprising:
(a) Passing a stream comprising a cyclic monoterpene to a dehydrogenation unit, wherein the stream is contacted with a dehydrogenation catalyst under conditions effective for the oxidation of the cyclic monoterpene to produce H₂ and a dehydrogenation-product stream comprising ρ-cymene;
(b) Separating the dehydrogenation-product stream of step (b) in a fractionation zone comprising at least one separation column to produce a fractionated ρ-cymene stream and a cyclic monoterpene stream;
(c) Combining the fractionated ρ-cymene stream of step (c) with benzene to produce a stream comprising benzene and ρ-cymene;
(d) Passing the stream comprising benzene and ρ-cymene of step (d) to a transalkylation unit, wherein the benzene and ρ-cymene is contacted with a transalkylation catalyst under conditions effective to produce a transalkylation-product stream comprising cumene;
(e) Separating the transalkylation-product stream of step (e) in a benzene separation column to produce a benzene-rich stream and a C₇₊-enriched stream;
(f) Separating the C₇₊-enriched stream of step (f) in a toluene separation column to produce a renewable toluene-enriched stream and a C₈₊-enriched stream;
(g) Separating the C₈₊-enriched stream of step (g) in a xylene separation column to produce a renewable xylene-enriched stream and a C₉₊-enriched stream;
(h) Separating the C₉₊-enriched stream of step (h) in a cumene separation column to produce a renewable cumene-enriched stream and C₁₀₊-enriched stream; and
(i) Separating the C₁₀₊-enriched stream of step (i) in a cymene separation column to produce a cymene-enriched stream and a C₁₀₊-enriched stream.
